SEQUENCE LISTING (92 sequences)

SEQ ID NO: 1 - protein, 6 aa, synthetic construct
  REGION 1..6: note = Description of sequence: LINKER
  VARIANT 1: note = Leucine may be either d-leucine or l-leucine
LEHELG

SEQ ID NO: 2 - protein, 6 aa, synthetic construct
  REGION 1..6: note = Description of sequence: LINKER
  VARIANT 1: note = Leucine may be either d-leucine or l-leucine
LEHALG

SEQ ID NO: 3 - protein, 6 aa, synthetic construct
  REGION 1..6: note = Description of sequence: LINKER
  VARIANT 1: note = Leucine may be either d-leucine or l-leucine
LEAELG

SEQ ID NO: 4 - protein, 6 aa, synthetic construct
  REGION 1..6: note = Description of sequence: LINKER
  VARIANT 1: note = Leucine may be either d-leucine or l-leucine
LEAALG

SEQ ID NO: 5 - protein, 6 aa, synthetic construct
  REGION 1..6: note = Description of sequence: LINKER
  VARIANT 1: note = Leucine may be either d-leucine or l-leucine
  VARIANT 3: note = X refers to any amino acid
LEXELG

SEQ ID NO: 6 - protein, 6 aa, synthetic construct
  REGION 1..6: note = Description of sequence: LINKER
  VARIANT 1: note = Leucine may be either d-leucine or l-leucine
  VARIANT 6: note = X refers to any amino acid
LEAELX

SEQ ID NO: 7 - protein, 7 aa, synthetic construct
  REGION 1..7: note = Description of sequence: LINKER
LEHELGN

SEQ ID NO: 8 - protein, 7 aa, synthetic construct
  REGION 1..7: note = Description of sequence: LINKER
  MOD_RES 1: note = Leucine is acetylated
  MOD_RES 7: note = N is bonded to a fluorescent probe} 7-amino-4-carbamoylmethylcoumarin
LEHELGN

SEQ ID NO: 9 - protein, 6 aa, synthetic construct
  REGION 1..6: note = Description of sequence: LINKER
  MOD_RES 1: note = Leucine is acetylated
  MOD_RES 6: note = G is bonded to a fluorescent probe} 7-amino-4-carbamoylmethylcoumarin
LEHELG

SEQ ID NO: 10 - protein, 5 aa, synthetic construct
  REGION 1..5: note = Description of sequence: LINKER
  MOD_RES 1: note = Leucine is acetylated
  MOD_RES 5: note = L is bonded to a fluorescent probe} 7-amino-4-carbamoylmethylcoumarin
LEHEL

SEQ ID NO: 11 - protein, 4 aa, synthetic construct
  REGION 1..4: note = Description of sequence: LINKER
  MOD_RES 1: note = Leucine is acetylated
  MOD_RES 4: note = E is bonded to a fluorescent probe} 7-amino-4-carbamoylmethylcoumarin
LEHE

SEQ ID NO: 12 - skipped

SEQ ID NO: 13 - protein, 4 aa, synthetic construct
  REGION 1..4: note = Description of sequence: LINKER
LEHE

SEQ ID NO: 14 - protein, 6 aa, synthetic construct
  REGION 1..6: note = Description of sequence: LINKER
WSPKYM

SEQ ID NO: 15 - protein, 6 aa, synthetic construct
  REGION 1..6: note = Description of sequence: LINKER
  MOD_RES 1: note = Tryptophan is acetylated
  MOD_RES 6: note = M is bonded to a fluorescent probe} 7-amino-4-carbamoylmethylcoumarin
WSPKYM

SEQ ID NO: 16 - protein, 11 aa, synthetic construct
  REGION 1..11: note = Description of sequence: LINKER
TPTDPPTTPPT

SEQ ID NO: 17 - protein, 5 aa, synthetic construct
  REGION 1..5: note = Description of sequence: LINKER
GGSGG

SEQ ID NO: 18 - protein, 5 aa, synthetic construct
  REGION 1..5: note = Description of sequence: LINKER
GGSAA

SEQ ID NO: 19 - skipped

SEQ ID NO: 20 - protein, 13 aa, synthetic construct
  REGION 1..13: note = Description of sequence: LINKER
GGTPTDPPTTPPT

SEQ ID NO: 21 - protein, 7 aa, synthetic construct
  REGION 1..7: note = Description of sequence: LINKER
ENLWSPD

SEQ ID NO: 22 - protein, 7 aa, synthetic construct
  REGION 1..7: note = Description of sequence: LINKER
YQSANAG

SEQ ID NO: 23 - protein, 7 aa, synthetic construct
  REGION 1..7: note = Description of sequence: LINKER
NVPEHGT

SEQ ID NO: 24 - protein, 6 aa, synthetic construct
  REGION 1..6: note = Description of sequence: Linker
AEHELG

SEQ ID NO: 25 - protein, 7 aa, synthetic construct
  REGION 1..7: note = Description of sequence: LINKER
SGIEMYN

SEQ ID NO: 26 - protein, 7 aa, synthetic construct
  REGION 1..7: note = Description of sequence: LINKER
LGVDPGS

SEQ ID NO: 27 - protein, 7 aa, synthetic construct
  REGION 1..7: note = Description of sequence: LINKER
ENTMQCC

SEQ ID NO: 28 - protein, 7 aa, synthetic construct
  REGION 1..7: note = Description of sequence: LINKER
HCAGFTP

SEQ ID NO: 29 - protein, 7 aa, synthetic construct
  REGION 1..7: note = Description of sequence: LINKER
HVADDQC

SEQ ID NO: 30 - protein, 7 aa, synthetic construct
  REGION 1..7: note = Description of sequence: LINKER
VQQNPCQ

SEQ ID NO: 31 - protein, 7 aa, synthetic construct
  REGION 1..7: note = Description of sequence: LINKER
NGACDAI

SEQ ID NO: 32 - protein, 7 aa, synthetic construct
  REGION 1..7: note = Description of sequence: LINKER
MLPDHGI

SEQ ID NO: 33 - protein, 7 aa, synthetic construct
  REGION 1..7: note = Description of sequence: LINKER
LGEVSAL

SEQ ID NO: 34 - protein, 7 aa, synthetic construct
  REGION 1..7: note = Description of sequence: LINKER
AVASDSV

SEQ ID NO: 35 - protein, 6 aa, synthetic construct
  REGION 1..6: note = Description of sequence: Linker
LAHELG

SEQ ID NO: 36 - protein, 7 aa, synthetic construct
  REGION 1..7: note = Description of sequence: LINKER
GDNQLHN

SEQ ID NO: 37 - protein, 7 aa, synthetic construct
  REGION 1..7: note = Description of sequence: LINKER
CSTLSPT

SEQ ID NO: 38 - protein, 7 aa, synthetic construct
  REGION 1..7: note = Description of sequence: LINKER
HFPENAQ

SEQ ID NO: 39 - protein, 7 aa, synthetic construct
  REGION 1..7: note = Description of sequence: LINKER
DEVNGEC

SEQ ID NO: 40 - protein, 7 aa, synthetic construct
  REGION 1..7: note = Description of sequence: LINKER
SADLNNQ

SEQ ID NO: 41 - protein, 7 aa, synthetic construct
  REGION 1..7: note = Description of sequence: LINKER
SALSNES

SEQ ID NO: 42 - protein, 7 aa, synthetic construct
  REGION 1..7: note = Description of sequence: LINKER
QAPAPTS

SEQ ID NO: 43 - protein, 7 aa, synthetic construct
  REGION 1..7: note = Description of sequence: LINKER
SANTPMT

SEQ ID NO: 44 - protein, 7 aa, synthetic construct
  REGION 1..7: note = Description of sequence: LINKER
DSYYSGF

SEQ ID NO: 45 - protein, 7 aa, synthetic construct
  REGION 1..7: note = Description of sequence: LINKER
STFDGGN

SEQ ID NO: 46 - protein, 7 aa, synthetic construct
  REGION 1..7: note = Description of sequence: LINKER
DGSVEMT

SEQ ID NO: 47 - protein, 6 aa, synthetic construct
  REGION 1..6: note = Description of sequence: LINKER
  MOD_RES 1: note = Leucine is acetylated
  MOD_RES 6: note = G is bonded to a fluorescent probe} 7-amino-4-carbamoylmethylcoumarin
AEHELG

SEQ ID NO: 48 - protein, 6 aa, synthetic construct
  REGION 1..6: note = Description of sequence: LINKER
  MOD_RES 1: note = Leucine is acetylated
  MOD_RES 6: note = G is bonded to a fluorescent probe} 7-amino-4-carbamoylmethylcoumarin
LAHELG

SEQ ID NO: 49 - protein, 6 aa, synthetic construct
  REGION 1..6: note = Description of sequence: LINKER
  MOD_RES 1: note = Leucine is acetylated
  MOD_RES 6: note = G is bonded to a fluorescent probe} 7-amino-4-carbamoylmethylcoumarin
LEAELG

SEQ ID NO: 50 - protein, 6 aa, synthetic construct
  REGION 1..6: note = Description of sequence: LINKER
  MOD_RES 1: note = Leucine is acetylated
  MOD_RES 6: note = G is bonded to a fluorescent probe} 7-amino-4-carbamoylmethylcoumarin
LEHALG

SEQ ID NO: 51 - protein, 6 aa, synthetic construct
  REGION 1..6: note = Description of sequence: LINKER
  MOD_RES 1: note = Leucine is acetylated
  MOD_RES 6: note = G is bonded to a fluorescent probe} 7-amino-4-carbamoylmethylcoumarin
LEHEAG

SEQ ID NO: 52 - protein, 6 aa, synthetic construct
  REGION 1..6: note = Description of sequence: LINKER
  MOD_RES 1: note = Leucine is acetylated
  MOD_RES 6: note = A is bonded to a fluorescent probe} 7-amino-4-carbamoylmethylcoumarin
LEHELA

SEQ ID NO: 53 - protein, 7 aa, synthetic construct
  REGION 1..7: note = Description of sequence: LINKER
DAVNGEC

SEQ ID NO: 54-56 - skipped (3 entries)

SEQ ID NO: 57 - protein, 6 aa, synthetic construct
  REGION 1..6: note = Description of sequence: LINKER
LEMELG

SEQ ID NO: 58 - protein, 6 aa, synthetic construct
  REGION 1..6: note = Description of sequence: LINKER
LEGELG

SEQ ID NO: 59 - protein, 6 aa, synthetic construct
  REGION 1..6: note = Description of sequence: LINKER
LECELG

SEQ ID NO: 60 - protein, 6 aa, synthetic construct
  REGION 1..6: note = Description of sequence: LINKER
LEDELG

SEQ ID NO: 61 - protein, 6 aa, synthetic construct
  REGION 1..6: note = Description of sequence: LINKER
LEFELG

SEQ ID NO: 62 - protein, 6 aa, synthetic construct
  REGION 1..6: note = Description of sequence: LINKER
LEEELG

SEQ ID NO: 63 - protein, 6 aa, synthetic construct
  REGION 1..6: note = Description of sequence: LINKER
LEHELG

SEQ ID NO: 64 - protein, 6 aa, synthetic construct
  REGION 1..6: note = Description of sequence: LINKER
LEIELG

SEQ ID NO: 65 - protein, 6 aa, synthetic construct
  REGION 1..6: note = Description of sequence: LINKER
LEKELG

SEQ ID NO: 66 - protein, 6 aa, synthetic construct
  REGION 1..6: note = Description of sequence: LINKER
LELELG

SEQ ID NO: 67 - protein, 6 aa, synthetic construct
  REGION 1..6: note = Description of sequence: LINKER
LENELG

SEQ ID NO: 68 - protein, 6 aa, synthetic construct
  REGION 1..6: note = Description of sequence: LINKER
LEQELG

SEQ ID NO: 69 - protein, 6 aa, synthetic construct
  REGION 1..6: note = Description of sequence: LINKER
LERELG

SEQ ID NO: 70 - protein, 6 aa, synthetic construct
  REGION 1..6: note = Description of sequence: LINKER
LESELG

SEQ ID NO: 71 - protein, 6 aa, synthetic construct
  REGION 1..6: note = Description of sequence: LINKER
LETELG

SEQ ID NO: 72 - protein, 6 aa, synthetic construct
  REGION 1..6: note = Description of sequence: LINKER
LEVELG

SEQ ID NO: 73 - protein, 6 aa, synthetic construct
  REGION 1..6: note = Description of sequence: LINKER
LEWELG

SEQ ID NO: 74 - protein, 6 aa, synthetic construct
  REGION 1..6: note = Description of sequence: LINKER
LEPELG

SEQ ID NO: 75 - protein, 6 aa, synthetic construct
  REGION 1..6: note = Description of sequence: LINKER
LEYELG

SEQ ID NO: 76 - skipped

SEQ ID NO: 77 - DNA, 135 bp, synthetic construct
  misc_feature 1..135: note = Description of sequence: gblock DNA sequence
  source 1..135: mol_type = other DNA
ttgttcctttctattctcacagtgcacttatgggcctgaacgatatttttgaagcgcagaaaattgaatggcatgaaggcggcagcctcgagtttgaagcgcagggtggtagtgcggccgcacatcatcatcacc

SEQ ID NO: 78 - protein, 52 aa, synthetic construct
  REGION 1..52: note = Description of sequence: gblock amino acid sequence
MGLNDIFEAQKIEWHEGGSLEFEAQGGSAAAHHHHHHGAAEQKLISEEDLNG

SEQ ID NO: 79 - protein, 26 aa, synthetic construct
  REGION 1..26: note = Description of sequence: Protein sequence containing Reverse complement of unique barcode
  VARIANT 11..17: note = x is any amino acid
IEWHEGGSGGXXXXXXXGGSAAAHHH

SEQ ID NO: 80 - DNA, 78 bp, synthetic construct
  misc_feature 1..78: note = Description of sequence: primer sequence
  source 1..78: mol_type = other DNA
  variation 30: note = n is adenosine cytosine guanosine or thymine
  variation 32..33: note = n is adenosine cytosine guanosine or thymine
  variation 35..36: note = n is adenosine cytosine guanosine or thymine
  variation 38..39: note = n is adenosine cytosine guanosine or thymine
  variation 41..42: note = n is adenosine cytosine guanosine or thymine
  variation 44..45: note = n is adenosine cytosine guanosine or thymine
tgatgatgatgtgcggccgcactaccaccnmnnmnnmnnmnnmnnmnnaccaccgctgccgccttcatgccattcaat

SEQ ID NO: 81 - skipped

SEQ ID NO: 82 - DNA, 54 bp, synthetic construct
  misc_feature 1..54: note = Description of sequence: Forward Primer
  source 1..54: mol_type = other DNA
gagttcagacgtgtgctcttccacactctttcccatggtgatgatgatgtgcgg

SEQ ID NO: 83 - DNA, 49 bp, synthetic construct
  misc_feature 1..49: note = Description of sequence: Reverse Primer
  source 1..49: mol_type = other DNA
aatgatacggcgaccaccgagatctacacgcagaaattgaatggcatga

SEQ ID NO: 84 - DNA, 50 bp, synthetic construct
  misc_feature 1..50: note = Description of sequence: Forward barcoding primer
  source 1..50: mol_type = other DNA
aatgatacggcgaccaccgagatctacacgcagaaaattgaatggcatga

SEQ ID NO: 85 - DNA, 60 bp, synthetic construct
  misc_feature 1..60: note = Description of sequence: Reverse Barcoding primer;; X = adenosine cytosine guanosine or thymine
  source 1..60: mol_type = other DNA
  variation 25..31: note = n is adenosine cytosine guanosine or thymine
caagcagaagacggcatacgagatnnnnnnngtgactggagttcagacgtgtgctcttcc

SEQ ID NO: 86 - skipped

SEQ ID NO: 87 - DNA, 18 bp, synthetic construct
  misc_feature 1..18: note = Description of sequence: Forward Primer
  source 1..18: mol_type = other DNA
aatgatacggcgaccacc

SEQ ID NO: 88 - DNA, 19 bp, synthetic construct
  misc_feature 1..19: note = Description of sequence: Reverse Primer
  source 1..19: mol_type = other DNA
caagcagaagacggcatac

SEQ ID NO: 89 - DNA, 39 bp, synthetic construct
  misc_feature 1..39: note = Description of sequence: Read1 Primer
  source 1..39: mol_type = other DNA
gaccaccgagatctacacgcagaaaattgaatggcatga

SEQ ID NO: 90 - DNA, 198 bp, synthetic construct
  misc_feature 1..198: note = Description of sequence: Barcode construct
  source 1..198: mol_type = other DNA
  variation 67..93: note = n is adenosine cytosine guanosine or thymine
  variation 168..174: note = n is adenosine cytosine guanosine or thymine
aatgatacggcgaccaccgagatctacacgcagaaaattgaatggcatgaaggcggcagcggtggtnnnnnnnnnnnnnnnnnnnnnnnnnnnggtggtagtgcggccgcacatcatcatcaccatgggaaagagtgtggaagagcacacgtctgaactccagtcacnnnnnnnatctcgtatgccgtcttctgcttg

SEQ ID NO: 91 - protein, 7 aa, synthetic construct
  REGION 1..7: note = Description of sequence: Linker
HVADDQG

SEQ ID NO: 92 - protein, 57 aa, synthetic construct
  REGION 1..57: note = Description of sequence: Gene 3 protein bound peptide
  MOD_RES 57: note = A is bound to gene 3 protein
  VARIANT 22..28: note = x is any amino acid
MGLNDIFEAQKIEWHEGGSGGXXXXXXXGGSAAAHHHHHHGAAEQKLISEEDLNGAA

(19) United States
(12) Patent Application Publication
(10) Pub. No.: US 2026/0355329 A1
(43) Pub. Date: Nov. 12, 2026

(54) PRODRUG LINKER COMPOSITIONS AND METHODS OF USE

(71) Applicant: Ohio State Innovation Foundation, Columbus, OH (US)

(72) Inventor: Jonathan H. BOYCE, Columbus, OH (US)

(21) Appl. No.: 19/485,369

(22) PCT Filed: May 17, 2024
(86) PCT No.: PCT/US2024/030018

§ 371 Date: Nov. 18, 2025

Related U.S. Application Data
(60) Provisional application No. 63/503,002, filed on May 18, 2023.
(60) Provisional application No. 63/503,300, filed on May 19, 2023.

(51) Int. Cl. A61K 47/64; A61K 47/54; C12N 15/10

(52) U.S. Cl. CPC A61K 47/64; A61K 47/542; A61K 47/545; C12N 15/1037

(57) ABSTRACT

Disclosed herein are protease-cleavable linkers and compositions thereof for use of treating and/or preventing diseases, including, but not limited to cancer. Also disclosed herein are methods of making and screening for protease-cleavable linkers.

### CROSS-REFERENCE TO RELATED APPLICATIONS

This PCT application claims priority to, and the benefit of, U.S. Provisional Patent Application No. 63/503,002, filed May 18, 2023, and U.S. Provisional Patent Application No. 63/503,300, filed May 19, 2023, entitled “METHODS TO SCREEN FOR PROTEASE-CLEAVABLE LINKERS, PROTEASE-CLEAVABLE LINKERS, AND RELATED COMPOSITIONS AND METHODS,” which are incorporated by reference herein in their entireties.

### STATEMENT REGARDING FEDERALLY SPONSORED RESEARCH

This invention was made with Government Support under Grant No. R35GM128656 awarded by the National Institutes of Health. The Government has certain rights in the invention.

### REFERENCE TO SEQUENCE LISTING

The sequence listing submitted on May 17, 2024, as an .XML file entitled “103361-473WO1_17MAY2024_2” created on May 17, 2024, and having a file size of 125,699 bytes is hereby incorporated by reference pursuant to 37 C.F.R. § 1.52 (e) (5).

### FIELD

The present disclosure relates protease-cleavable linkers, compositions, and methods of use thereof for treating and/or preventing diseases, including, but not limited to cancer and bacterial infections. Also disclosed herein are methods of making and screening for protease-cleavable linkers.

### BACKGROUND

Patients with aggressive types of cancers such as, for example, pancreatic cancer, metastatic triple negative breast cancer, and metastatic triple negative breast cancer tend to have limited options to ensure long-term survival. The identification, diagnosis, and treatment for these and other types of cancers is critical. When surgery is not beneficial, such as in advanced pancreatic cancer, the only option is often traditional chemotherapy, and the side effects reduce patient quality of life. Proper diagnosis of the cancer subtype can also be challenging, which can be essential for proper treatment. Histopathological methods, currently the standard for differentiating between subtypes, often fail to differentiate between subtypes with similar features and phenotypes. Despite improvements in diagnostic tools and imaging, differentiating between subtypes remains a challenge with current methods, and an accurate diagnosis is essential for proper treatment and improved outcomes.

Pharma has largely withdrawn from antibiotic discovery, and more people will die from bacterial infections than cancer by the year 2050. Gram-negative (G−) pathogens are largely responsible for the world's nosocomial infections. They are more difficult to treat than Gram-positive (G+) pathogens due to an outer membrane (OM) and OM efflux pumps that aid resistance against antibiotics that would otherwise be effective. As a result, the arsenal of G− therapies could be expanded if G+ antibiotics are repurposed to overcome these barriers. As more infections emerge that are resistant to all current therapies, innovation in this area is direly needed.

Given limitations of current therapeutics and diagnostic methods for cancers and antimicrobial infections, there is need to address the aforementioned problems mentioned above by developing platforms, compositions, and methods for improving diagnostic and treatment practices. The platforms, compositions, and methods disclosed herein address these and other needs.

### SUMMARY

The present disclosure provides methods of screening for a protease-cleavable linker. The present invention also provides methods of purifying compositions comprising the protease-cleavable linker. The present invention also provides compounds and/or compositions comprising a protease-cleavable linker.

In one aspect, disclosed herein is a method of screening for a protease-cleavable linker, the method comprising: a) a negative selection process comprising: i) incubating a substrate display library comprising one or more peptide sequences in a first complex biological matrix, ii) generating a first cleaved phage-containing peptide sequence and an uncleaved phage-containing peptide sequence, and iii) separating the uncleaved phage-containing peptide sequence from the first cleaved phage-containing peptide sequence; b) a positive selection process comprising: i) contacting the uncleaved phage-containing peptide sequence with a second complex biological matrix, and ii) producing a second cleaved phage-containing peptide sequence; c) identifying a first nucleic acid sequence of the first cleaved phage-containing peptide sequence and identifying a second nucleic acid sequence of the second cleaved phage-containing peptide sequence, d) comparing the first and second nucleic acid sequences to identify one or more protease-cleavable linkers; and e) performing at least two iterations of steps a) through d).

In some embodiments, less than 10 iterations of steps a) through d) are performed. In some embodiments, 2, 3, 4, 5, 6, 7, 8, or 9 iterations of steps a) through d) are performed.

In some embodiments, the first complex biological matrix comprises healthy blood serum or plasma, healthy organ tissue homogenates, media comprising healthy organ tissue homogenates, healthy organoid homogenates derived from healthy cells, conditioned media comprising healthy organoids derived from healthy cells, healthy cell lines, conditioned media comprised of healthy cell lines, or combinations thereof.

In some embodiments, the second complex biological matrix comprises cancer tissue homogenates, media comprising cancer tissue homogenates, cancer organoid homogenates, conditioned media comprising cancer organoid homogenates, malignant ascites, malignant cystic fluid, cancer cell lines, conditioned media comprising cancer cell lines, or combinations thereof. In some embodiments, the second complex biological matrix comprises periplasmic extracts isolated from Gram-negative bacteria, bacteria lysates from Gram-positive or Gram-negative bacteria, or combinations thereof.

In some embodiments, the negative selection process comprises a control cell or a microenvironment comprising the control cell. In some embodiments, the positive selection process comprises a cell of interest or a microenvironment comprising the cell of interest. In some embodiments, the cell of interest comprises a Gram-negative bacterium or a Gram-positive bacterium. In some embodiments, the gram-negative bacterium comprises A. nosocomialis, P. aeruginosa, E. cloacae, E. coli, A. baumannii, E. aerogenes, K. pneumonia, S. typhi, H. influenzae, L. pneumophilia, V. cholerae, Y. enterocolitica, B. pertussis, B. melitensis, H. pylori, S. dysenteriae, C. jejuni, M. catarrhalis, B. cepacia, or S. enterica. In some embodiments, the gram-positive bacterium comprises S. aureus, S. epidermidis, S. pneumoniae, S. pyogenes, C. difficile, C. perfringens, or L. monocytogenes. In some embodiments, the cell of interest comprises a cancer cell. In some embodiments, the cancer cell comprises hepatocellular carcinoma, cholangiocarcinoma, combined hepatocholangiocarcinoma, ovarian carcinoma, uterine carcinoma, triple negative breast cancer, glioblastoma, or pancreatic carcinoma.

In some embodiments, the cell of interest is either the same or different in the at least two iterations of steps a) through e).

In some embodiments, the first and second nucleic acid sequences encode the one or more protease-cleavable linkers. In some embodiments, the first and second nucleic acid sequence is identified using Next Generation Sequencing (NGS). In some embodiments, the method further comprises identifying cleavage kinetics in the presence of the first complex biological matrix and the second complex biological matrix.

In one aspect, disclosed herein is a compound comprising a targeting moiety, a therapeutic agent, and a cleavable linker, wherein the cleavable linker comprises a peptide sequence selected from SEQ ID NO: 1, SEQ ID NO: 2, SEQ ID NO: 3, SEQ ID NO: 4, SEQ ID NO: 5, SEQ ID NO: 6, or a chiral variant thereof.

In some embodiments, the cleavable linker is directly or indirectly linked to the targeting moiety and the therapeutic agent. In some embodiments, the targeting moiety comprises a siderophore.

In some embodiments, the targeting moiety comprises an HMM siderophore, an enterobactin, an aerobactin, a yersiniabactin, or derivatives thereof. In some embodiments, the HMM siderophore comprises Compound I

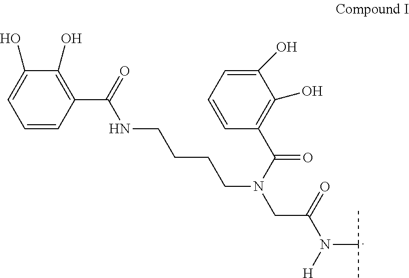

In some embodiments, the enterobactin comprises Compound II

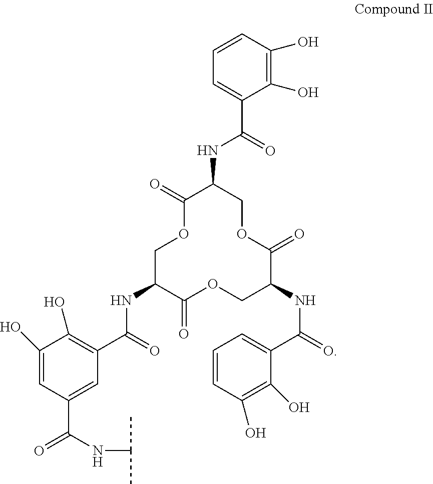

In some embodiments, the aerobactin comprises Compound III

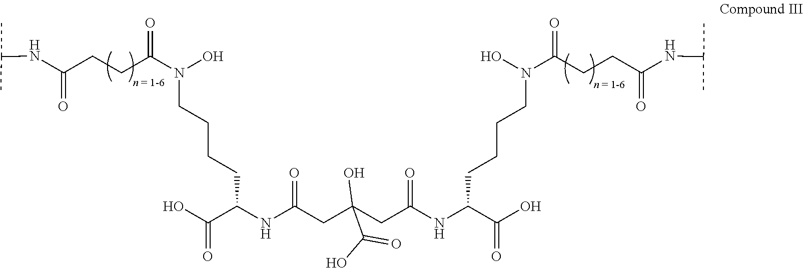

In some embodiments, the versiniabactin comprises Compound IV

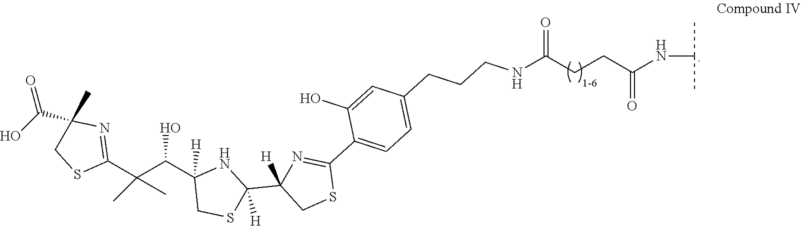

In some embodiments, the therapeutic agent comprises an antibiotic. In some embodiments, the antibiotic targets a cytoplasmic protein, a periplasmic protein, or a bacterial membrane protein. In some embodiments, the antibiotic comprises an amine- or aniline-containing antibiotic. In some embodiments, the antibiotic comprises a macrolide antibiotic, oxazolidinone antibiotic, cyclic lipopeptide antibiotic, or fluoroquinolone antibiotic. In some embodiments, the antibiotic comprises solithromycin, daptomycin, or eperezolid.

In some embodiments, the therapeutic agent comprises an anti-cancer chemotherapeutic agent. In some embodiments, the anti-cancer chemotherapeutic agent comprises an apoptosis agent, an anti-mitotic agent, a kinase inhibitor, an antimetabolite, an alkylating agent, paclitaxel, docetaxel, a bifunctional proteolysis-targeting chimera, or a combination thereof. In some embodiments, the anti-cancer chemotherapeutic agent comprises doxorubicin, mitomycin, or a combination thereof.

### BRIEF DESCRIPTION OF FIGURES

The accompanying figures, which are incorporated in and constitute a part of this specification, illustrate several aspects described below.

FIG. 1 shows the biopanning campaign workflow to optimize linkers that cleave selectively in each PLC subtype for protease-activated diagnostic development. Negative selections incorporate desired stability. Positive selections incorporate desired sensitivity.

FIG. 2 shows the validation of FRET diagnostic for each PLC subtype following sequence scoring from the phage platform.

FIG. 3 shows the siderophore-chemotherapeutic conjugates (SCCs) and mechanism of proteolytic release inside Gram-negative bacteria. (1) An intact SCC, with a serum-stable peptide linker reaches the site of infection and (2,3) is actively transported to the periplasm via outer membrane proteins. (4) Proteolysis of the conjugate's linker by periplasmic proteases that reside in the periplasm will release the chemotherapeutic payload. (5) The payload would then be free to diffuse through the inner membrane and bind to its targets in the cytoplasm.

FIG. 4 shows the workflow for discovering serum-stable linkers that cleave in periplasmic extract. Step 1. Substrate phage display with negative and positive selections. Step 2. NGS, data analysis, and candidate scoring. Step 3. FRET pair and ACC conjugate synthesis and comparison of cleavage kinetics.

FIG. 5 shows the peptide candidates with improved cleavage rates in periplasmic extract relative to serum. 25 out of 28 peptides synthesized showed improved cleavage rates in periplasmic extract relative to serum, which validated the selection criteria. AB-5075, AB-WC487, and AB-Nav18 are A. baumannii periplasmic extracts.

FIGS. 6A, 6B, and 6C show cleavage rates and comparisons of cleavage linkers. FIG. 6A shows the cleavage rate of a candidate linker in serum and periplasmic extract. MCA=7-methoxycoumarin-4-acetic acid; DNP=2,4-dinitrophenyl. FIGS. 6B and 6C show the comparison of linker cleavage in 50% mouse serum and periplasmic extract over 8 hours. FIG. 6B evaluates a linker that was discovered from phage platform with negative selections. FIG. 6C evaluates a linker that was discovered from phage platform without negative selections.

FIG. 7 shows that the DAVNGEC (SEQ ID NO: 53) peptide demonstrated a 21-fold improvement in periplasmic extract versus serum.

FIG. 8 shows that the A. baumannii extracts show better cleavage than non-A. baumannii extracts.

FIG. 9 shows the ACC conjugates synthesized to identify an optimal scarless linker for LEHELGN (SEQ ID NO: 7).

FIG. 10 shows the comparison between FRET pair cleavage and ACC release for LEHELGN (SEQ ID NO: 7).

FIGS. 11A, 11B, 11C, 11D, 11E, and 11F show the scarless release of ACC in serum and A. baumannii periplasmic extract.

FIG. 12 shows the cleavage rates of alanine-substituted Ac-LEHELG-ACC (SEQ ID NO: 9) variants over 8 hours in mouse serum and in periplasmic extracts from six different A. baumannii strains. ACC is a turn-on fluorescent probe that only fluoresces if it is cleaved without an amino acid scar. ACC excitation maximum: 355 nm. ACC emission maximum: 460 nm.

FIG. 13 shows the cleavage rates (μM/hour) of each Ac-LEXELG (SEQ ID NO: 5)-ACC peptide (where X represents 20 natural amino acids) in different periplasmic extracts of A. baumannii bacteria compared to 50% mouse serum over the course of 8 hours.

FIG. 14 shows the cell viability assay in HL60 cells and shows the cleavable LEHELG (SEQ ID NO: 1)-mitomycin C conjugate is non-toxic relative to mitomycin C.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### TERMINOLOGY

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100%, or more, increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

A “nucleotide” is a compound consisting of a nucleoside, which consists of a nitrogenous base and a 5-carbon sugar, linked to a phosphate group forming the basic structural unit of nucleic acids, such as DNA or RNA. The four types of DNA nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The word “vector” refers to any vehicle that carries a polynucleotide into a cell for the expression of the polynucleotide in the cell. The vector may be, for example, a phagemid, a plasmid, a virus, a phage particle, or a nanoparticle. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation.

As used herein, a “phagemid” refers to an engineered DNA-based cloning vector comprising both bacteriophage and plasmid properties and/or structures, such as for example a phagemid can comprise an origin of replication derived from a plasmid and an origin of replication derived from a bacteriophage. Phagemid vectors are engineered as hybrid of filamentous phages and plasmids to generate a product that can be grown as a plasmid, and also packaged as a single stranded DNA in viral particles.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins, and compositions comprising peptides, polypeptides, and proteins. As used herein, a polypeptide and/or protein is defined as a polymer of amino acids, typically of length ≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’). The variant polypeptide can also be a chiral variant of the parent polypeptide, wherein the chiral variant is a mirror image of the parent and cannot be superimposed on the parent. In some embodiments, the chiral variant and parent polypeptides are “enantiomers” of one another; they are often distinguished as either “right-handed or (D)” or “left-handed or (L)”.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide. A variant polypeptide may have substantially the same functional activity as a reference polypeptide.

The term “screening” refers to a method especially used in drug discovery in which data processing/control software, liquid handling devices, and sensitive detectors can allow for quick conductions of chemical, genetic, or pharmacological tests. This process allows one to quickly recognize active compounds, antibodies, genes and the like that modulate a particular biomolecular pathway. The results of these processes provide starting points for drug design or therapeutic treatments.

### Compounds Containing Protease-Cleavable Linkers

The present disclosure also provides compounds and/or compositions comprising a protease-cleavable linker. The present disclosure also provides methods of purifying compositions comprising the protease-cleavable linker.

In one aspect, disclosed herein is a compound comprising a targeting moiety, a therapeutic agent, and a cleavable linker, wherein the cleavable linker comprises a peptide sequence selected from L-LEHELG (SEQ ID NO: 1), L-LEHALG (SEQ ID NO: 2), L-LEAELG (SEQ ID NO: 3), L-LEAALG (SEQ ID NO: 4), D-LEHELG (SEQ ID NO: 1), D-LEHALG (SEQ ID NO: 2), D-LEAELG (SEQ ID NO: 3), D-LEAALG (SEQ ID NO: 4), L-LEXELG (SEQ ID NO: 5), D-LEXELG (SEQ ID NO: 5), L-LEAELX (SEQ ID NO: 6), or D-LEAELX (SEQ ID NO: 6), wherein X is any chiral or achiral amino acid.

In some embodiments, the cleavable linker is directly linked to the targeting moiety and the therapeutic agent. In some embodiments, the cleavable linker is indirectly linked to the targeting moiety. In some embodiments, the targeting moiety comprises a siderophore. In some embodiments, the targeting moiety comprises an HHM siderophore, enterobactin, or yersiniabactin.

In some embodiments, the HMM siderophore comprises Compound I

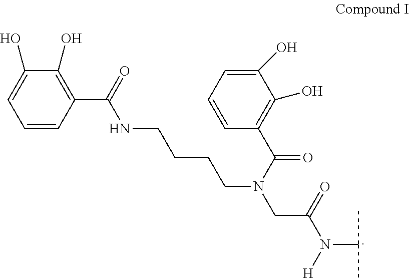

In some embodiments, the enterobactin comprises Compound II

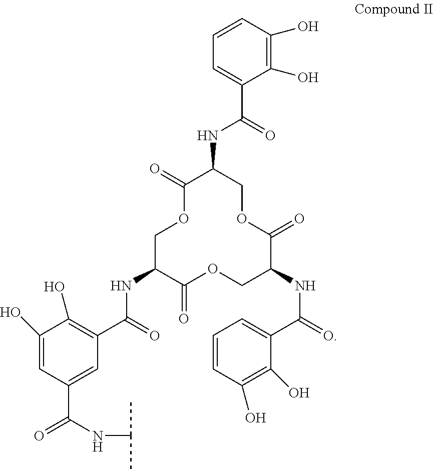

In some embodiments, the aerobactin comprises Compound III

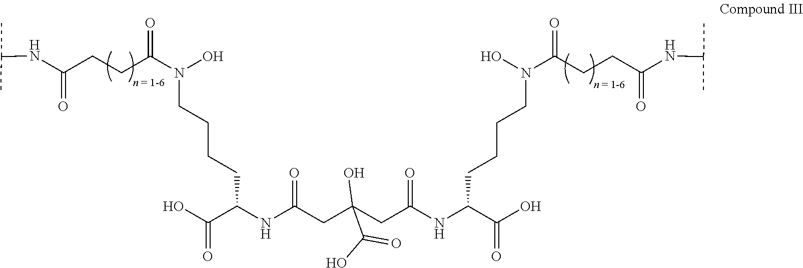

In some embodiments, the yersiniabactin comprises Compound IV

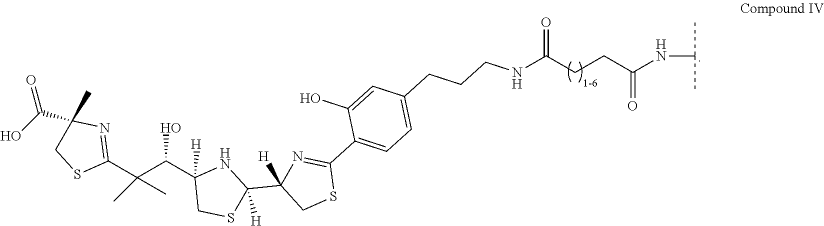

In some embodiments, the therapeutic agent comprises an antibiotic. In some embodiments, the antibiotic targets a cytosolic molecule, a periplasmic molecule, or a bacterial membrane molecule. In some embodiments, the antibiotic comprises a basic amine-containing antibiotic. In some embodiments, the antibiotic comprises an amine- or aniline-containing antibiotic. In some embodiments, the antibiotic includes but is not limited to solithromycin, penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, ceftazidime, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination.

In some embodiments, the therapeutic agent comprises an anti-cancer chemotherapeutic agent. In some embodiments, the anti-cancer chemotherapeutic agent comprises an apoptosis agent, an anti-mitotic agent, a kinase inhibitor, an antimetabolite, an alkylating agent, paclitaxel, docetaxel, a bifunctional proteolysis-targeting chimera, or a combination thereof. In some embodiments, the anti-cancer chemotherapeutic agent includes, but is not limited to doxorubicin, mitomycin, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. verteporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, raltitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Novartis), XL765 (Sanofi Aventis), PF-4691502 (Pfizer), GDC0980 (Genentech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, hexamethyl melamine, or any combinations thereof.

### Methods of Screening for Protease-Cleavable Linkers

The present disclosure also provides methods of identifying and screening protease-cleavable linkers for drug development.

In one aspect, disclosed herein is a method of screening for a protease-cleavable linker, the method comprising: a) a negative selection process comprising: i) incubating a substrate display library comprising one or more peptide sequences in a first complex biological matrix, ii) generating a first cleaved phage-containing peptide sequence and an uncleaved phage-containing peptide sequence, and iii) separating the uncleaved phage-containing peptide sequence from the first cleaved phage-containing peptide sequence; b) a positive selection process comprising: i) contacting the uncleaved phage-containing peptide sequence with a second complex biological matrix, and ii) producing a second cleaved phage-containing peptide sequence; c) identifying a first nucleic acid sequence of the first cleaved phage-containing peptide sequence and identifying a second nucleic acid sequence of the second cleaved phage-containing peptide sequence, d) comparing the first and second nucleic acid sequences to identify one or more protease-cleavable linkers; and e) performing at least two iterations of steps a) through d).

In one aspect, disclosed herein is a method of screening for two or more protease-cleavable linkers, the method comprising: a) a negative selection process comprising: i) incubating a substrate display library comprising one or more peptide sequences in a first complex biological matrix, ii) generating a first cleaved phage-containing peptide sequence and an uncleaved phage-containing peptide sequence, and iii) separating the uncleaved phage-containing peptide sequence from the first cleaved phage-containing peptide sequence; b) a positive selection process comprising: i) contacting the uncleaved phage-containing peptide sequence with a second complex biological matrix, and ii) producing a second cleaved phage-containing peptide sequence; c) identifying a first nucleic acid sequence of the first cleaved phage-containing peptide sequence and identifying a second nucleic acid sequence of the second cleaved phage-containing peptide sequence, d) comparing the first and second nucleic acid sequences to identify one or more protease-cleavable linkers; and e) performing at least two iterations of steps a) through d).

In some embodiments, less than 10 iterations of steps a) through d) are performed. In some embodiments, 2, 3, 4, 5, 6, 7, 8, or 9 iterations of steps a) through d) are performed. In some embodiments, the method screens 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more protease-cleavable linkers.

As used herein, a “phagemid” refers to a type of cloning vector engineered to be a hybrid of a bacteriophage and a plasmid to produce a vector that can grow as a plasmid and also be packaged as single stranded DNA in viral particles. It should be noted that the phagemid of any preceding aspect can comprise one or more components from a bacteriophage, one or more components from a plasmid, or any combination thereof, deemed necessary to achieved the desired function. In some embodiments, the phagemid of any preceding aspect comprises an origin of plasmid replication, an origin of bacteriophage replication, or a combination thereof.

In some embodiments, the first complex biological matrix comprises healthy blood serum or plasma, healthy organ tissue homogenates, media comprising healthy organ tissue homogenates, healthy organoid homogenates derived from healthy cells, conditioned media comprising healthy organoids derived from healthy cells, healthy cell lines, conditioned media comprised of healthy cell lines, or combinations thereof.

In some embodiments, the second complex biological matrix comprises cancer tissue homogenates, media comprising cancer tissue homogenates, cancer organoid homogenates, conditioned media comprising cancer organoid homogenates, malignant ascites, malignant cystic fluid, cancer cell lines, conditioned media comprising cancer cell lines, or combinations thereof. In some embodiments, the second complex biological matrix comprises periplasmic extracts isolated from Gram-negative bacteria, bacteria lysates from Gram-positive or Gram-negative bacteria, or combinations thereof.

In some embodiments, the method comprises a negative selection process and a positive selection process. In some embodiments, the negative selection process comprises a control cell or a microenvironment comprising the control cell. In some embodiments, the positive selection process comprises a cell of interest or a microenvironment comprising the cell of interest.

In some embodiments, the cell of interest comprises a gram-negative bacterium. In some embodiments, the gram-negative bacterium comprises A. nosocomialis, P. aeruginosa, E. cloacae, E. coli, A. baumannii, E. aerogenes, K. pneumonia, S. typhi, H. influenzae, L. pneumophilia, V. cholerae, Y. enterocolitica, B. pertussis, B. melitensis, H. pylori, S. dysenteriae, C. jejuni, M. catarrhalis, B. cepacia, or S. enterica. In some embodiments, the cell of interest comprises a gram-positive bacterium including, but not limited to S. aureus, S. epidermidis, S. pneumoniae, S. pyogenes, C. difficile, C. perfringens, or L. monocytogenes.

In some embodiments, the cell of interest comprises a cancer cell. In some embodiments, the cancer cell is derived from a cancer including, but not limited to hepatocellular carcinoma (HCC), biliary cancer (such as, for example cholangiocarcinoma (CC)), combined hepatocholangiocarcinoma (cHCC-CC), ovarian carcinoma, uterine carcinoma, triple negative breast cancer, pancreatic carcinoma, glioblastoma, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendocrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the cell of interest is the same in the at least two iterations of steps a) through e). In some embodiments, the cell of interest is different in the at least two iterations of steps a) through e).

In some embodiments, the first and second nucleic acid sequences encode the one or more protease-cleavable linkers. In some embodiments, the first and second nucleic acid sequence is identified using Next Generation Sequencing (NGS). In some embodiments, the method further comprises identifying cleavage kinetics in the presence of the first complex biological matrix and the second complex biological matrix.

Methods of Preparing and/or Purifying Therapeutic Compositions

In one aspect, disclosed herein is a method of purifying a therapeutic composition comprising a targeting moiety, a therapeutic agent, and a cleavable linker, the method comprising washing the therapeutic composition with about 1% acetic acid/acetonitrile, about 10 mM ammonium acetate, or a combination thereof, binding the therapeutic composition to a reverse-phase column comprising about 0.1% acetic acid, wherein the column is a component on a liquid chromatography apparatus, and eluting the therapeutic composition from the column using a solution comprising 5% to 65% acetonitrile in water.

In some embodiments, the method further comprises repeating the eluting step of any preceding aspect. In some embodiments, the solution comprises 5% to 47% acetonitrile in water. In some embodiments, the method further comprises purifying the therapeutic composition using high performance liquid chromatography.

In some embodiments, the method of purifying a therapeutic composition of any preceding aspect comprising a cleavable linker comprising L-LEHELG (SEQ ID NO: 1), L-LEHALG (SEQ ID NO: 2), L-LEAELG (SEQ ID NO: 3), L-LEAALG (SEQ ID NO: 4), D-LEHELG (SEQ ID NO: 1), D-LEHALG (SEQ ID NO: 2), D-LEAELG (SEQ ID NO: 3), D-LEAALG (SEQ ID NO: 4), L-LEXELG (SEQ ID NO: 5), D-LEXELG (SEQ ID NO: 5), L-LEAELX (SEQ ID NO: 6), or D-LEAELX (SEQ ID NO: 6), wherein X is any chiral or achiral amino acid.

In some embodiments, the therapeutic composition comprises a siderophore, L-LEXELG (SEQ ID NO: 5), and mitomycin C. In some embodiments, the therapeutic composition comprises a siderophore, D-LEXELG (SEQ ID NO: 5), and mitomycin C. In some embodiments, the X of SEQ ID NO: 5 is alanine, methionine, glycine, cysteine, aspartic acid, phenylalanine, glutamic acid, histidine, isoleucine, lysine, leucine, asparagine, glutamic acid, arginine, serine, threonine, valine, tryptophan, proline, or tyrosine.

In some embodiments, LEXE (SEQ ID NO: 76) is critical composition for serum stability. In some embodiments, LEXELG (SEQ ID NO: 5) comprises optimized properties for cleavage rate and serum stability. In some embodiments, variants of LEXELG (SEQ ID NO: 5) comprise LEMELG (SEQ ID NO: 57), LEAELG (SEQ ID NO: 3), LEGELG (SEQ ID NO: 58), LECELG (SEQ ID NO: 59), LEDELG (SEQ ID NO: 60), LEFELG (SEQ ID NO: 61), LEEELG (SEQ ID NO: 62), LEHELG (SEQ ID NO: 63), LEIELG (SEQ ID NO: 64), LEKELG (SEQ ID NO: 65), LELELG (SEQ ID NO: 66), LENELG (SEQ ID NO: 67), LEQELG (SEQ ID NO: 68), LERELG (SEQ ID NO: 69), LESELG (SEQ ID NO: 70), LETELG (SEQ ID NO: 71), LEVELG (SEQ ID NO: 72), LEWELG (SEQ ID NO: 73), LEPELG (SEQ ID NO: 74), or LEYELG (SEQ ID NO: 75).

A number of embodiments of the disclosure have been described. Nevertheless, it will be understood that various modifications may be made without departing from the spirit and scope of the invention. Accordingly, other embodiments are within the scope of the following claims. By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Platform Applications to Pancreatic Cancer

Despite improvements in cancer treatments in the clinic, pancreatic cancer is one of the deadliest and most difficult cancers to treat. It is projected that by 2030, pancreatic cancer will be the second leading cause of cancer-related deaths. Early diagnosis is challenging due to the cancer's progress being slow and asymptomatic. Surgery, radiation therapy, and chemotherapy are treatments that may extend survival or relieve symptoms, however, fewer than 20% of patients are candidates for surgical intervention due to metastasis. Immunotherapy (i.e. antibody drug conjugates (ADC)) remains an option for only a small number of cases and is normally ineffective due to the size of the ADC and the conjugate's inability to effectively penetrate the cancer stroma microenvironment. The tumor microenvironment of pancreatic cancers is heterogeneous with multiple proteases contributing to cancer progression and metastasis.

The present disclosure provides a method of developing selective cleavable linkers and their incorporation into targeted chemotherapeutics for pancreatic cancers that leverages the differences in protease populations between the tumor microenvironment and non-tumorous or healthy microenvironments. Because traditional chemotherapy is still dependent on toxic compounds that lack selectivity, their side effects reduce patient quality of life and lead to life-threatening complications. One method of mitigating chemotherapeutic toxicity is through the attachment of a peptide linker, which prevents cell permeability and/or target binding. Ideally, the peptide is cleaved by a protease that is upregulated within or proximal to tumor cells, releasing the active drug in the location of interest. However, even the most selective linkers [e.g. Valine-Citrulline (Val-Cit)], used in FDA-approved antibody-drug conjugates (ADCs) for immunotherapy, undergo premature drug release causing neutropenia as a major side effect. The fact that only three different protease-cleavable linkers exist on the market is testament to the lack of innovative and effective strategies for cleavable linker discovery. In addition, cleavable peptide prodrugs (without an antibody or targeting moiety) have not been FDA approved because existing protease-cleavable linkers lack the necessary selectivity to ensure site-specific delivery. Therefore, improvements in linker selectivity are drastically needed. One inherent limitation is that linkers are traditionally designed to target one or two proteases that are upregulated in cancer cells, and it is challenging to target a single protease because the human genome codes for more than 600 proteases, many with overlapping specificities. Furthermore, a single protease may not be present in enough concentration for prodrug activation. To simplify the challenge of optimizing a protease-activated peptide linker, the differences in overall protease activity between cancerous and healthy microenvironments is leveraged by screening complex biological mixtures (e.g. healthy and cancerous patient-derived tissue homogenates in addition to conditioned media from organoid cultures) with substrate phage display (SPD). The platform benefits from a highly diverse 7-amino acid peptide phage library (containing up to 20\hat{ }7 variants) and from the ability to remove peptides from the library that cleave in healthy microenvironments (negative selections) and identify remaining sequences that are cleaved selectively in tumor microenvironments (positive selections). By leveraging these differences through the substrate phage platform disclosed herein, selective linkers are discovered that would cleave in proximity to cancerous tissue, leading to the release of a chemotherapeutic payload in a site-specific manner.

Each biopanning campaign consists of multiple rounds of selection (positive and negative selections, along with biological replicates), and cancerous samples deriving from different patient populations may be used to increase the probability of site-specific payload release in a broad range of patients. Homogenized pancreatic cancer tissues, malignant cystic fluids, homogenized necrosectomy samples, or conditioned media from cancerous organoid cultures can be used for positive selections. Serum, homogenized healthy tissues (e.g. liver and kidney), or conditioned media from healthy organoid cultures can be used for negative selections to optimize linker stability in healthy microenvironments. The phagemids from the selections are barcoded and sequenced by Next Generation Sequencing (NGS), and the sequences are scored based on positive/negative enrichment as described in Example 5. Synthesis of fluorescence resonance energy transfer (FRET) pairs and ACC conjugates containing candidate linkers enable the evaluation of cleavage kinetics and identification of candidates for site-specific payload release in the various samples used in the biopanning selections (e.g. serum, tissue homogenates, and organoid conditioned media). Candidate linkers can be conjugated to chemotherapeutics, and controls can be synthesized to interrogate on-target activity and prodrug selectivity (e.g. non-cleavable linker variants).

SPD was originally developed as a method to profile the substrate preferences of a single protease. Prior to the disclosure reported herein, SPD had never been successfully utilized for the development of cleavable prodrug linkers. Previous efforts by Whitney et al. to use SPD resulted in linkers that had serum instability and cleaved in healthy tissue extracts, which deprioritized prodrug applications. Recent efforts by Boyce et al. to utilize SPD for prodrug linkers led to candidates that were unstable in mouse and human serum, preventing application in an animal model. In addition to SPD, there are a number of other proteolytic profiling technologies that screen libraries of protease substrates (e.g. multiplex substrate profiling by mass spectrometry (MSP-MS), positional scanning of synthetic combinatorial libraries, and indexed arrays of fluorogenic substrates). Compared to other protease-profiling methods, SPD enables the evaluation of maximal substrate diversity, with greater than 10⁹ variants. Importantly, all existing proteolytic profiling technologies, including SPD, were developed for single protease evaluations or comparisons of individual proteases and were not designed to interrogate the substrate profiles of complex biological mixtures with numerous proteases. However, a phage-based platform is described that enables identification of linker candidates that cleave selectively in one set of microenvironments while remaining stable in others, allowing for the discovery of highly selective prodrug linkers.

The platform described is non-obvious because the proteases and their cleavage preferences are unknown with multiple cleavage sites for every enriched candidate identified, which complicates the discovery of consensus sequences. Indeed, for most applications of SPD, knowledge of the protease or identification of common cleavage motifs among multiple substrates is a critical component of the data analysis process. In contrast, this approach depends on the ability to identify sequences that are cleaved in one mixture over others independent of the proteases or their cleavage preferences. An SPD platform was developed by incorporating negative selections (to remove linkers from a diverse linker library that cleave in healthy tissue homogenates and serum) and positive selections (to identify remaining sequences that cleave in cancerous tissue homogenates, malignant pancreatic cystic fluid, and conditioned media from organoid cultures). Importantly, NGS analysis of both positive and negative selections are critical for identification of highly selective candidates and minimizing false positives. Targeting multiple proteases may minimize resistance to prodrug therapeutics by increasing the evolutionary cost required to prevent conjugate activation through protease elimination. This platform is leveraged to achieve optimal stability and cleavage selectivity of discovered linkers for prodrug and diagnostic development.

It should be understood that the platforms and methods of the present disclosure can further comprise the following steps or features:
1) Separate biopanning campaigns incorporating positive selections conducted with primary cancer cells and/or metastatic cancer cells at various stages of progression;
2) Peptide prodrugs have potential to benefit patients that do not respond well to immunotherapy.
3) For effective linkers discovered in this platform, attachment of antibodies as targeting groups (e.g. those used in marketed antibody-drug conjugates) can be incorporated. Current linkers on the market undergo off-target release resulting in neutropenia; the linkers discovered by this platform have the potential to overcome this limitation.

### Acquisition of Tissue Homogenates for Linker Discovery Platform:

### Description of Procedure

The 6-8 week-old C57BL/6 mice (both male and female mice with a gender ratio of 1:1) are used to isolate necessary organs for homogenization to be used in selection studies. The mouse is euthanized prior to organ harvest by CO₂ inhalation followed by cervical dislocation, which is consistent with the recommendations of the Panel on Euthanasia of the American Veterinary Medical Association. This method employs a top-opening chamber, charged with CO₂ prior to introducing the animal(s). After the animal(s) are placed in the chamber, a slow flow of CO₂ is continued for a few minutes to maintain a high concentration at the bottom of the chamber. After breathing has stopped and the animal(s) are unconscious, euthanasia is completed by cervical dislocation.

The euthanized BL/6 mouse is placed on its back, and an incision is made with small surgical scissors. Relevant organs (e.g. kidney and liver) are removed using forceps and scissors. The organs are then processed on ice using the back of a 10-ml syringe plunger. The resulting fluid is filtered through a cell strainer and diluted in phosphate-buffered saline (PBS). Centrifugation facilitates removal of residual cell debris, followed by filtration through a 0.2 μm filter. The samples are then aliquoted into eight Eppendorf tubes for use in biopanning selections.

Applications in mice utilizes healthy BL/6 mice to sample diverse organs for use in negative selections of the substrate phage platform to improve the probability of identifying linkers that are stable in healthy mouse microenvironments. Human tissue homogenates are procured through the Total Cancer Care (TCC) Program at Ohio State University. Those animals exhibiting clinical signs of disease or are severely lethargic, unresponsive to stimuli, have excessive weight loss (greater than 40% compared to other mice in this healthy cohort) or are unable to right themselves, are removed immediately and humanely euthanized.

### Example 2: Platform Applications to Liver Cancer

Patients with unresectable primary liver cancer (PLC) have limited options to ensure long-term survival. The diagnosis of unresectable PLC subtypes (e.g. hepatocellular carcinoma (HCC), cholangiocarcinoma (CC), and combined hepatocellular-cholangiocarcinoma (cHCC-CC)) is critical for proper treatment, which varies for each subtype. HCC patients benefit from targeted therapy, immunotherapy, and antiviral agents, while CC patients respond to traditional chemotherapy in addition to targeted therapy, and immunotherapy. cHCC-CC patients have the poorest prognosis and mainly rely on major hepatectomy for optimal management; other treatments include radiation, yttrium-90 radioembolization, chemotherapy, combined radiation and chemotherapy, combined surgery and chemotherapy, and triple therapy (surgery, radiation, and chemotherapy). Histopathological methods, currently the standard for differentiating the subtypes, often fail to identify cHCC-CC due to similarities between HCC and CC. Despite improvements in diagnostic tools and imaging, differentiating the cHCC-CC subtype remains a challenge with current methods, and an accurate diagnosis is essential for proper treatment and improved outcomes. As a result, patient care could be improved if a diagnostic is developed to overcome these barriers.

In addition to improved diagnostics, the present disclosure provides a method of developing selective cleavable linkers for incorporation into targeted chemotherapeutics for PLC that leverages the differences in protease populations between the tumor microenvironment and non-tumorous or healthy microenvironments. Because chemotherapy is still dependent on toxic compounds that lack selectivity, their side effects reduce patient quality of life and can lead to life-threatening complications. One method of mitigating chemotherapeutic toxicity is through the attachment of a peptide linker, which prevents cell permeability and/or target binding. Ideally, the peptide is cleaved by a protease that is upregulated within or proximal to tumor cells, releasing the active drug in the location of interest. Indeed, even the most selective linkers [e.g. Valine-Citrulline (Val-Cit)] in FDA-approved antibody-drug conjugates (ADCs) for immunotherapy undergo premature drug release causing neutropenia as a major side effect. The fact that only three different protease-cleavable linkers exist on the market is testament to the lack of innovative and effective strategies for cleavable linker discovery. In addition, cleavable peptide prodrugs (without an antibody or targeting moiety) have not been FDA approved because existing protease-cleavable linkers lack the necessary selectivity to ensure site-specific delivery. Therefore, improvements are drastically needed. One inherent limitation is that linkers are traditionally designed to target one or two proteases. However, it is challenging to target a single protease because the human genome codes for more than 600 proteases, and a single protease may not be present in enough concentration for prodrug activation.

To simplify the challenge of optimizing a protease-activated peptide linker, the differences in overall protease activity between cancerous and healthy microenvironments is leveraged by screening complex biological mixtures (e.g. conditioned media from organoids, patient-tissue homogenates, etc.) with SPD. The platform benefits from a highly diverse 7-amino acid peptide phage library (containing up to 20\hat{ }7 variants) and from the ability to remove peptides from the library that cleave in healthy microenvironments (negative selections) and identify remaining sequences that are cleaved selectively in tumor microenvironments (positive selections). By leveraging these differences, linkers may be developed that are either subtype-selective (e.g. cleave in one PLC subtype) or subtype-inclusive (e.g. cleave in three PLC subtypes) for the development of protease-activated PLC diagnostics and targeted chemotherapeutics.

The present disclosure provides a method of developing a diagnostic for rapidly identifying cHCC-CC, CC, and HCC subtypes from a biopsied sample. Using this method, three distinct protease-activated turn-on fluorescent probes could be developed that specifically cleave in a homogenate of each PLC subtype. The method comprises at least three separate biopanning campaigns (one for each PLC subtype), and each campaign consists of multiple rounds of selection and biological replicates. PLC tissue homogenates deriving from different patient populations may improve the probability that protease-activated linkers discovered from this platform are effective for a broad range of patients. The phagemids from the selections can be barcoded and sequenced by NGS, and the sequences can be scored based on positive/negative enrichment as described in Example 5. Synthesis of FRET pairs and evaluation of cleavage kinetics in HCC, CC, and cHCC-CC homogenates would inform which linkers provide optimal protease-activated diagnostics for each PLC subtype.

The present disclosure provides a method of developing protease-activated chemotherapeutic prodrugs that target all three subtypes of PLC: HCC, CC, and cHCC-CC.

The phage platform disclosed herein can be conducted with conditioned media from organoids or patient tissue homogenates that derive from each subtype. Serum, conditioned media from organoids, and/or tissue homogenates derived from healthy tissue (e.g. liver and kidney) can be used for negative selections to optimize linker stability in healthy microenvironments. Following biopanning selections, NGS, and sequence scoring, the sequences can be synthesized as FRET pairs and optimized for scarless prodrug release using turn-on fluorescent probes as described in Example 5. Candidate linkers can be conjugated to chemotherapeutics, and controls can be synthesized to interrogate on-target activity and prodrug selectivity. The potency of chemotherapeutic prodrugs and controls can be investigated in cell-viability assays utilizing healthy (kidney, liver) and cancerous organoid models (HCC, CC, and cHCC-CC).

The present disclosure combines SPD with tissue homogenates and organoids to address limitations that have prevented the ability to diagnose PLC subtypes. The protease-activated imaging agents developed may improve the ability to diagnose and study cHCC-CC cases and facilitate further advances in interventional surgical imaging and monitoring therapeutic response (34). Currently, HCC patients (80% of PLC cases) tend to experience higher toxicity from traditional chemotherapy treatments [e.g. doxorubicin- and gemcitabine-based therapies, FOLFOX (5-fluorouracil, leucovorin, oxaliplatin), and PIAF (cisplatin/interferon alpha-2b/doxorubicin/5-fluorouracil)], with minimal benefit to overall survival (33-36). In contrast, chemotherapy remains the first-line standard-of-care for most patients with CC (15% of PLC cases). Chemotherapy use for cHCC-CC patients (5% of PLC cases) is understudied because it is rare and challenging to diagnose due to similarities with HCC and/or CC. Chemotherapeutic peptide prodrugs, developed by the platform described herein, may enable patients with unresectable PLC to tolerate higher doses by increasing the therapeutic index and improve long-term survival. This strategy has the potential to benefit patients with any PLC subtype. Additionally, the peptide prodrugs developed by the platform described herein may especially benefit those patients that do not respond well to immunotherapy.

Protease dysregulation in cancers often contributes to invasion and tumor spread. The protease mixtures in each tumor microenvironment have complex but specific proteolytic signatures that differ from those in healthy microenvironments. SPD is used to screen these microenvironments and identify their preferred proteolytic substrates. The phage libraries incorporate up to 20\hat{ }7 unique peptide variants, which have the benefit of providing a large number of enriched candidates (e.g. sequences with desired cleavage properties), enhancing the probability of discovering shared substrates across all PLC subtypes that may lead to broad-spectrum PLC-targeting prodrugs. At the same time, the large number of enriched candidates also aid the discovery of unique substrates that cleave selectively in one PLC subtype over others, which may result in protease-activated imaging agents that are effective for PLC subtype diagnoses.

The platform described is non-obvious because the proteases and their cleavage preferences are unknown with multiple cleavage sites for every enriched candidate identified, which complicates the discovery of consensus sequences. Indeed, for most applications of SPD, knowledge of the protease or identification of common cleavage motifs among multiple substrates is a critical component of the data analysis process. In contrast, this approach depends on the ability to identify sequences that are cleaved in one mixture over others independent of the proteases or their cleavage preferences. An SPD platform was developed by incorporating negative selections (to remove linkers from a diverse linker library that cleave in healthy tissue homogenates, organoid conditioned media, and serum) and positive selections (to identify remaining sequences that cleave in PLC tissue homogenates and tumoroid conditioned media). Importantly, NGS analysis of both positive and negative selections are critical for identification of highly selective candidates and minimizing false positives. Targeting multiple proteases may minimize resistance to prodrug therapeutics by increasing the evolutionary cost required to prevent conjugate activation through protease elimination. This platform is leveraged to achieve optimal stability and cleavage selectivity of discovered linkers for prodrug and diagnostic development.

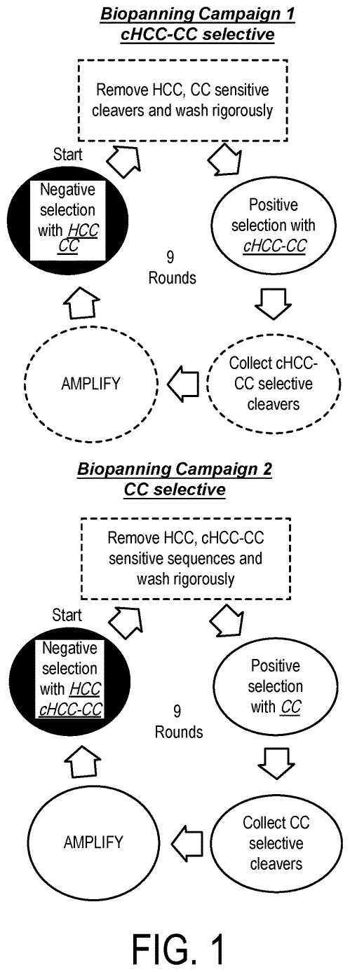

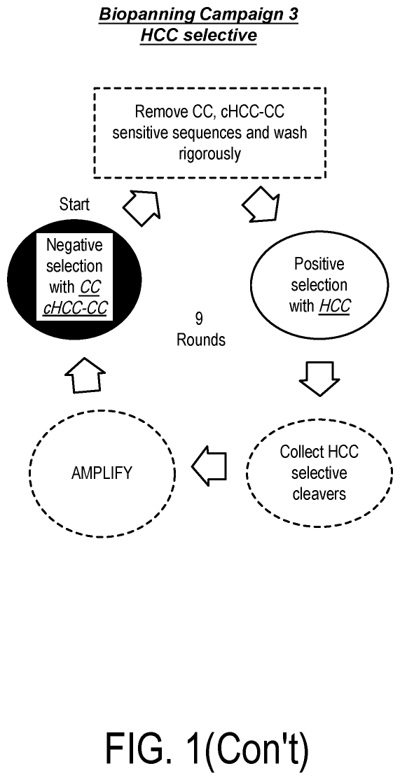

The present disclosure provides a method of developing a diagnostic that can rapidly identify cHCC-CC, CC, and HCC subtypes from a biopsied sample. Application of the method describe herein would result in three unique protease-activated turn-on fluorescent probes that specifically cleave in a homogenate of each PLC subtype. Linker selectivity can be optimized with the phage platform described in Example 5, using positive and negative selections in combination with NGS sequencing. The selections incorporate PLC-subtype homogenates. The homogenates can be prepared on ice from 2 mg of tissue per 100 μL of phosphate-buffered saline (PBS) containing 1 mM ZnCl₂. To optimize a linker for each PLC subtype, separate biopanning campaigns can be conducted, each consisting of multiple rounds of selection, biological replicates, and PLC samples deriving from different patient populations. The biopanning campaigns begin with a randomized amino acid phage library and are summarized as follows. Biopanning Campaign 1 (FIG. 1): To identify sequences that release fluorescent probes in cHCC-CC homogenates with stability in HCC and CC homogenates, at least three rounds of SPD can be conducted with negative selections incorporating HCC and CC homogenates and positive selections incorporating cHCC-CC homogenates. The negative selections remove sequences from the randomized amino acid phage library that cleave in HCC and CC homogenates, and the remaining sequences are subjected to positive selections for the identification of sequences that cleave selectively in cHCC-CC homogenates. Biopanning Campaign 2 (FIG. 1): To identify sequences that rapidly release fluorescent probes in CC homogenates with stability in cHCC-CC and HCC homogenates, at least three rounds of SPD can be conducted with negative selections incorporating cHCC-CC and HCC homogenates and positive selections incorporating CC homogenates. Biopanning Campaign 3 (FIG. 1): To identify sequences that rapidly release fluorescent probes in HCC homogenates with stability in cHCC-CC and CC homogenates, at least three rounds of SPD can be conducted with negative selections incorporating cHCC-CC and CC homogenates and positive selections incorporating HCC homogenates. Each of the three biopanning campaigns described above can be optionally repeated with the following variables: 1) a separately prepared randomized amino acid phage library (see Methods Example 5 for phage library preparation), and 2) utilizing tissue homogenates from diverse patient populations (e.g. Asian, African, African American, Caucasian American, and Pacific Islander descent) [ideally, all three biopanning campaigns described above are repeated for each population and with a combination of homogenates from multiple populations]. The phagemids from the selections (positive and negative) can be sequenced by NGS, and the sequences scored based on positive/negative enrichment as described herein.

Python scripts enable scoring of the sequences, and a selection of sequences with excellent scores can be synthesized as FRET pairs. Four Python scripts are used in the scoring process: The first Python script converts DNA sequences to amino acid sequences for each sample. The second script compares positive and negative selection read counts for each sequence from a single round. The third script allows for comparison of the positive and negative selection counts across all rounds from each biopanning campaign. Information from the third Python script is analyzed to create a focused candidate list based on positive/negative enrichment rankings and relative read counts in positive and negative selections across all rounds from each campaign. The fourth script allows for scoring of each candidate as specified in Example 5.

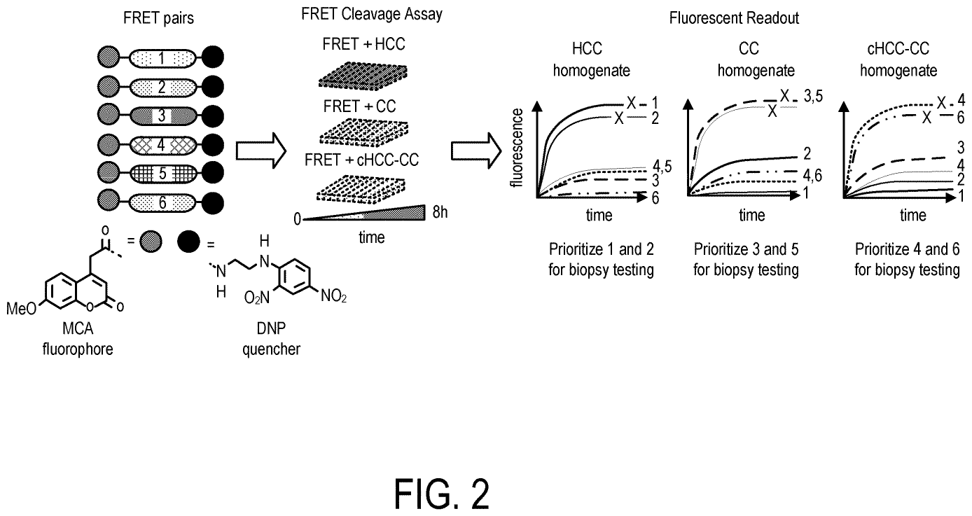

The cleavage kinetics of each FRET substrate can be assessed in HCC, CC, and cHCC-CC homogenates by monitoring for fluorescence over the course of 8 hours (FIG. 2). The peptides that display the highest sensitivity and selectivity for a specific PLC subtype can be selected as protease-activated diagnostic candidates. After candidate linkers have been selected for each subtype, their sensitivity can be tested using homogenates of biopsied samples deriving from primary liver cancer patients to aid in the identification of PLC subtype. The conclusion from the assay results can be compared to the sample's histopathological classification.

A method to improve protease-activated agents to aid in the identification of cHCC-CC subtypes of PLC is contemplated. The diagnostic is contemplated to be used in parallel with histopathological methods to provide further confidence of subtype, which is often a challenging distinction that is critical for proper treatment.

The platform described herein is also contemplated to generate protease-activated chemotherapeutic prodrugs that target all three subtypes of PLC: HCC, CC, and cHCC-CC. The homogenates from multiple PLC subtypes can be combined for positive selections in each round. FRET pairs that display the highest selectivity for HCC, CC, and cHCC-CC proteolysis relative to healthy homogenate samples can be synthesized as ACC conjugates (with variable linker lengths) to optimize for scarless drug release as described in Example 5. The scarless linkers can then be conjugated to chemotherapeutics using known chemical methods. Controls can be synthesized to interrogate on-target activity and probe of mechanism of action. For example, cleavable (L-amino acid linker) and non-cleavable (D-amino acid linker) variants can be synthesized for comparison to determine if cytotoxicity is due to proteolytic cleavage.

It should be understood that the platforms and methods of the present disclosure can further comprise the following steps or features:
1) Separate biopanning campaigns incorporating positive selections conducted with primary cancer cells and/or metastatic cancer cells at various stages of progression;
2) Peptide prodrugs have potential to benefit patients that do not respond well to immunotherapy.
3) For effective linkers discovered in this platform, attachment of antibodies as targeting groups (e.g. those used in marketed antibody-drug conjugates) can be incorporated. Current linkers on the market undergo off-target release resulting in neutropenia; the linkers discovered by this platform have the potential to overcome this limitation.

### Example 3: Platform Applications to Ovarian Cancer

The present disclosure provides a method of developing peptide prodrug linkers that cleave selectively in ovarian cancer ascites and relevant ovarian cancer cell lines or organoid models. Ideally, linkers would display rapid cleavage in ovarian cancer ascites and malignant cystic fluids and stability in the presence of serum and diverse healthy tissue homogenates, including kidney and liver. The method would incorporate positive and negative selections of SPD, followed by NGS sequencing, and sequence validation as described in Examples 2 and 5. The overarching goal of this refinement is to aid in the development of protease-activated prodrugs that target ovarian-cancer.

For protease-activated prodrug therapies, linker stability and selectivity remain a challenge. FDA approved protease-activated cancer treatments (e.g. ADCs) only incorporate three different linkers, and these medications are known to cause neutropenia as a common side effect due to premature drug release. This disclosure addresses these challenges through a platform that discovers highly selective protease-cleavable linkers.

To discover linkers for ovarian cancer, positive selections may include ovarian cancer ascites, malignant cystic fluid, conditioned media from ovarian cancer cell lines or organoids (e.g. OVCAR-3, OVCAR-4, OVCAR-5, OVCAR-8, NCI/ADR-RES, and SK-OV-3), etc. Negative selections may include, but are not limited to, serum and healthy tissue homogenates. Candidate linkers can be identified and validated as described in Example 5.

It should be understood that the platforms and methods of the present disclosure can further comprise the following steps or features:
1) Separate biopanning campaigns incorporating positive selections conducted with primary cancer cells and/or metastatic cancer cells at various stages of progression;
2) Peptide prodrugs have potential to benefit patients that do not respond well to immunotherapy.
3) For effective linkers discovered in this platform, attachment of antibodies as targeting groups (e.g. those used in marketed antibody-drug conjugates) can be incorporated. Current linkers on the market undergo off-target release resulting in neutropenia; the linkers discovered by this platform have the potential to overcome this limitation.

### Example 4: Platform Applications to Triple-Negative Breast Cancer

Triple-negative breast cancer (TNBC) constitutes approximately 15-20% of all the breast cancer cases and is characterized by the absence of estrogen receptor (ER), progesterone receptor (PR), and human epidermal growth factor receptor 2 (HER2) protein expression. Because TNBC cells lacks these receptors, targeted therapies are less effective and the primary treatment options rely on traditional chemotherapy. The absence of precise target selectivity and the toxicity caused by chemotherapy pose challenges in treatment and limit the potential for enhanced outcomes, despite the overall effectiveness of chemotherapy. Furthermore, resistance to chemotherapeutics account for 90% treatment failure in mTNBC patients. Alternative therapies, such as antibody-drug conjugates (ADCs), are limited for mTNBC with only one being FDA-approved (Trodelvy, 2021).

The present disclosure provides a method of developing improved targeted therapies for mTNBC through discovery of highly selective linkers for protease-activated chemotherapeutics. Chemotherapeutic peptide prodrugs may enable the mTNBC patient to tolerate higher doses of chemotherapeutics by increasing the therapeutic index and increasing long-term survival. In addition to benefiting mTNBC patients that undergo chemotherapy regimens, prodrugs that result from this platform may especially benefit those patients that do not respond well to immunotherapy.

To simplify the challenge of optimizing a linker, the present disclosure leverages the differences in overall protease activity between cancerous and healthy microenvironments by screening complex biological mixtures with SPD. The platform benefits from a highly diverse peptide phage library and from the ability to remove peptides from the library that cleave in healthy microenvironments (negative selections) and identify remaining sequences that are cleaved selectively in tumor microenvironments (positive selections). Indeed, by incorporating negative selections with mouse serum into the phage platform, a linker (LEHELGN (SEQ ID NO: 7)) was discovered to be 66-fold more stable in mouse serum than A. baumannii extract (see Example 5). The linker was then used to convert chemotherapeutics into potent antibiotic prodrugs with minimal toxicity in human cell lines.

To discover prodrug linkers that cleave in mTNBC ascites and patient-derived TNBC tissue homogenates (containing metastasized cancer tissue in the lungs, liver, and bone marrow and/or primary breast cancer tissue) but remain stable in the presence of serum and diverse healthy tissue homogenates (e.g. heart, kidney, liver, lung, bone marrow, and breast), negative and positive selections can be conducted utilizing the platform described in Example 5. In addition to tissue homogenates, conditioned media from 2D and 3D cultures can be incorporated into positive selections. Following NGS, the sequences can be scored and validated as described in Example 5.

It should be understood that the platforms and methods of the present disclosure can further comprise the following steps or features:
1) Separate biopanning campaigns incorporating positive selections conducted with primary cancer cells and/or metastatic cancer cells at various stages of progression;
2) Peptide prodrugs have potential to benefit patients that do not respond well to immunotherapy.
3) For effective linkers discovered in this platform, attachment of antibodies as targeting groups (e.g. those used in marketed antibody-drug conjugates, such as Trodelvy) can be incorporated. Current linkers on the market undergo off-target release resulting in neutropenia; the linkers discovered by this platform have the potential to overcome this limitation.

### Example 5: Platform Applications to Infectious Diseases and Linker Compositions

Siderophore-antibiotic conjugates (SACs) are touted as one strategy for repurposing G⁺ antibiotics for G-bacteria. Bacteria produce siderophores to sequester Fe (III), an essential nutrient. SACs evolved in bacteria as defenses against other nearby microbes. In the case of G⁻ bacteria, OM proteins enable SACs to actively transport to the periplasm (and in some species proceed to the cytoplasm), after which the conjugate may bind to its periplasmic targets. However, most antibiotics have targets in the cytoplasm and would require a cleavable linker because the siderophore and peptide linker interfere with inner-membrane (IM) permeability and/or target binding. Despite progress for SACs with non-cleavable linkers using periplasmic-targeting antibiotics (e.g., lipopeptides, β-lactams), development of cleavable linkers has stalled. Existing cleavable SACs are limited by premature or poor linker cleavage. While SACs have been extensively explored to repurpose G⁺ antibiotics for G⁻ bacteria, only one marketed drug [Fetroja (cefiderocol) with a non-cleavable linker] has resulted from two decades of research.

Protease-activated prodrugs incorporate amino acid linkers, providing one strategy to develop a selective cleavable linker for SACs. Previous amino acid linkers developed were unstable in mouse and human serum. Herein, a novel phage-based method is described to overcome this limitation through the discovery of a peptide linker that maintains high stability in mouse serum with rapid cleavage in bacteria. In contrast to traditional proteolytic profiling methods that identify substrate preferences for individual proteases, a method is described that leverages differences in protease activity between complex biological mixtures, each representing microenvironments for host or pathogen. For example, serum and tissue homogenates would represent microenvironments of the host, and periplasmic extracts would represent the periplasm of G⁻ bacteria. Analogous to a single protease, each biological mixture has a unique proteolytic signature because of the combined activity from associated proteases. This strategy enables identification of a linker that cleaves selectively in one set of microenvironments while remaining stable in others.

SPD was originally developed as a method to profile the substrate preferences of a single protease. Prior to the disclosure reported herein, SPD had never been successfully utilized for the development of cleavable prodrug linkers. Previous efforts by Whitney et al. to use SPD resulted in linkers that had serum instability and cleaved in healthy tissue extracts, which deprioritized prodrug applications. Recent efforts by Boyce et al. to utilize SPD for prodrug linkers led to candidates that were unstable in mouse and human serum, preventing application in an animal model. In addition to SPD, there are a number of other proteolytic profiling technologies that screen libraries of protease substrates (e.g. multiplex substrate profiling by mass spectrometry (MSP-MS), positional scanning of synthetic combinatorial libraries, and indexed arrays of fluorogenic substrates). Compared to other protease-profiling methods, SPD enables the evaluation of maximal substrate diversity, with greater than 10⁹ variants. Importantly, all existing proteolytic profiling technologies, including SPD, were developed for single protease evaluations or comparisons of individual proteases and were not designed to interrogate the substrate profiles of complex biological mixtures with numerous proteases. However, a phage-based platform is described that enables identification of linker candidates that cleave selectively in one set of microenvironments while remaining stable in others, allowing for the discovery of highly selective prodrug linkers.

The platform described is non-obvious because the proteases and their cleavage preferences are unknown with multiple cleavage sites for every enriched candidate identified, which complicates the discovery of consensus sequences. Indeed, for most applications of SPD, knowledge of the protease or identification of common cleavage motifs among multiple substrates is a critical component of the data analysis process. In contrast, this approach depends on the ability to identify sequences that are cleaved in one mixture over others independent of the proteases or their cleavage preferences.

Proteases in the periplasm, a compartment that contains >20 known proteases for protein quality control, are targeted to ensure localization of SAC and target protease, as all SACs pass through the periplasm, including those that eventually proceed to cytoplasm. Targeting multiple proteases minimizes antimicrobial resistance by increasing the evolutionary cost required to prevent conjugate activation. The platform described enabled the discovery of a linker that rapidly cleaved in a broad range of MDR pathogens, while maintaining stability in mouse serum. When incorporated into SACs, the linker enabled controlled and effective release of antibiotic warheads in nine G⁻ strains (Table 1).

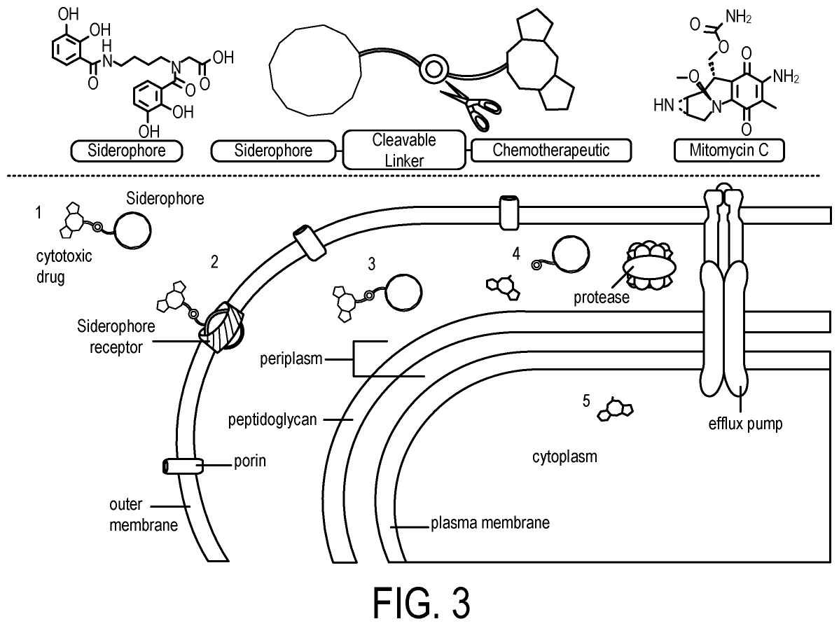

To identify sequences that cleave in the bacterial periplasm, periplasmic extracts were isolated for conducting biopanning selections. This increases the probability of releasing the drug inside the periplasm, and after release from the peptide, the drug (e.g. chemotherapeutic or antibiotic) is free to diffuse through the inner membrane and bind to its target(s) in the cytoplasm. FIG. 3 outlines the process by which a siderophore-chemotherapeutic conjugate is used to facilitate drug delivery into G⁻ bacteria. To improve the probability of discovering linkers that are stable enough to reach the site of infection in an animal model, negative selections with mouse serum were incorporated in the substrate phage platform. Negative selections remove sequences from the phage library that cleave in mouse serum, and the remaining sequences would presumably be serum stable. Positive selections with periplasmic extract would follow and enable the identification of sequences that are serum stable and cleave in a desired bacterial periplasmic environment.

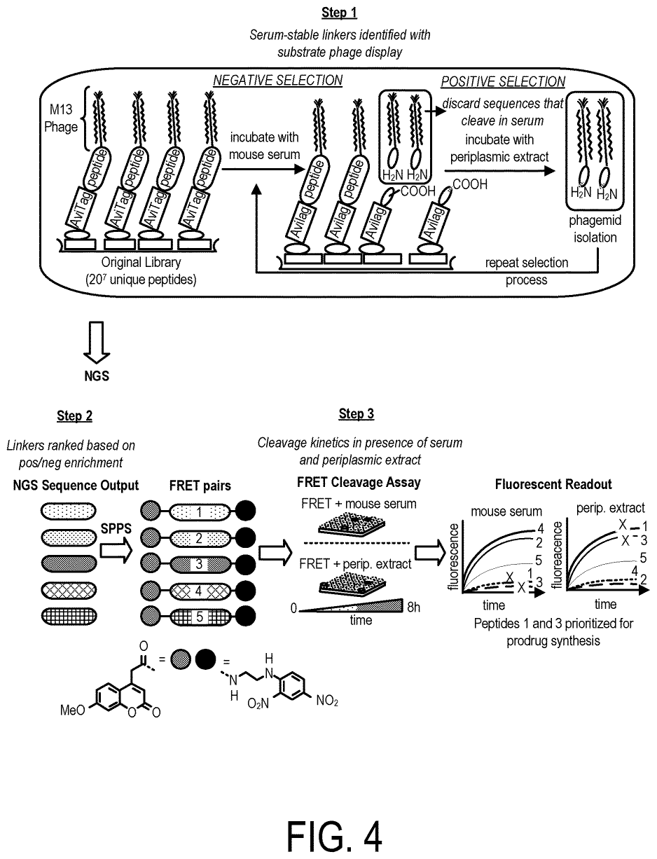

Starting with a randomized heptapeptide phage library (containing up to 20\hat{ }7 unique sequences), negative selections with mouse serum and positive selections with periplasmic extract were incorporated into each round, and the process was repeated for up to nine rounds of selection for each of six strains of multidrug resistant A. baumannii [A. baumannii WC487 (AB-WC487), A. baumannii Nav-18 (AB-Nav18), A. baumannii BC5 (AB-BC5), A. baumannii ATCC BAA-1797 (AB-1797), and A. baumannii 3-137 (AB-3-137)]. Each round of selection included (FIG. 4, step 1): 1) biotinylation of the AviTag, 2) immobilization of the phage library on streptavidin-coated 96-well plates 3) incubation with serum at 37 °C (negative selection), 4) incubation with periplasmic extract at 37 °C (positive selection), 5) amplification of phage-containing cleaved sequences from positive selections (evaluation of titers at this stage), and 6) phage purification and continuation with the next round of selection. Washes were implemented after steps 2 and 3 to remove sequences that stick to surfaces.

To enhance sensitivity of cleaved phage sequences per round, the stringency of cleavage conditions in periplasmic extract was increased with each succeeding round by reducing extract concentration (e.g. Total Protein: 1.0 mg/mL to 0.5 mg/mL) and incubation time (e.g. 3 h→2 h→1 h→30 min). Biological replicates were also incorporated. The replicates showed a strong correlation (R²=0.69-0.92) with good reproducibility. This is not a perfect correlation because NGS only samples a small portion (~10⁵⁻⁶) due to the high diversity of the starting library (>10⁹). The following controls were also evaluated for comparison of NGS sequencing data: 1) positive selections (without negative selections) for at least one A. baumannii strain and serum (4 rounds each), 2) wash controls (PBS or Tris instead of periplasmic extract) for titer comparison to verify enrichment, and 3) log-phase and stationary-phase periplasmic extracts were investigated to determine which type would provide more optimized sequences. Log-phase extracts provided higher enrichment, showing improved protease activity, and provided different enriched sequences than stationary-phase extracts. Therefore, log-phase extracts were used for the selection process.

For each positive and negative selection, the phagemid DNA was isolated from phage-infected TG-1 cells (infected with the cleaved phage sequences from one selection) using a ThermoScientific GeneJET Plamid Miniprep Kit. The DNA insert containing the randomized portion of the library was amplified by PCR, purified by DNA gel extraction, and barcoded with PCR using a Kapa HiFi PCR Kit. The samples were submitted for NGS (SE_50). Sequences had an overall 92% high-quality read count, and each sample contained 5-10 million sequence reads. The original library (with maximal diversity) was also sequenced for comparison to calculate sequence enrichment. NGS of both the negative and positive selections enabled scoring of the sequences based on their relative read counts in periplasmic extract and mouse serum throughout all rounds of selection using customized Python scripts (FIG. 4, step 2). The sequences were prioritized as described in the Methods (NGS and Data Analysis). The data pointed to more than 700 candidate sequences, and 28 sequences were selected for synthesis as FRET pairs.

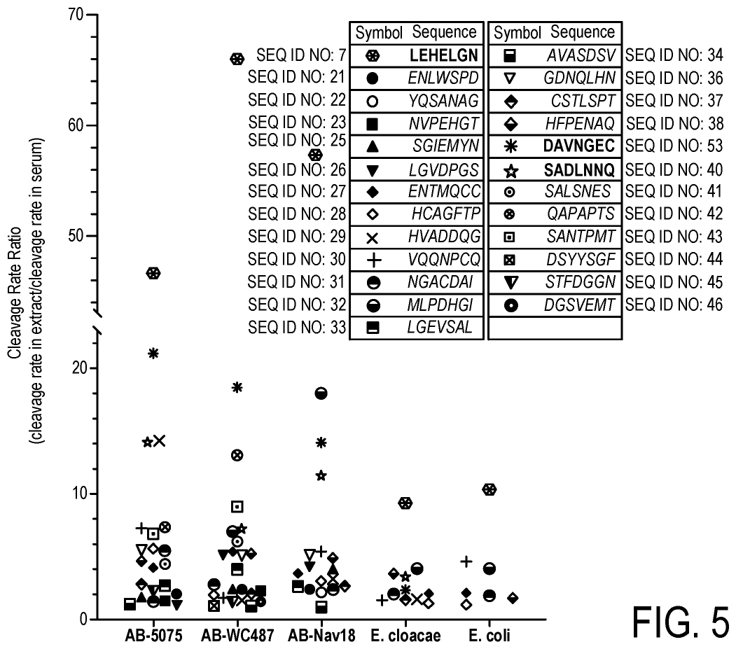

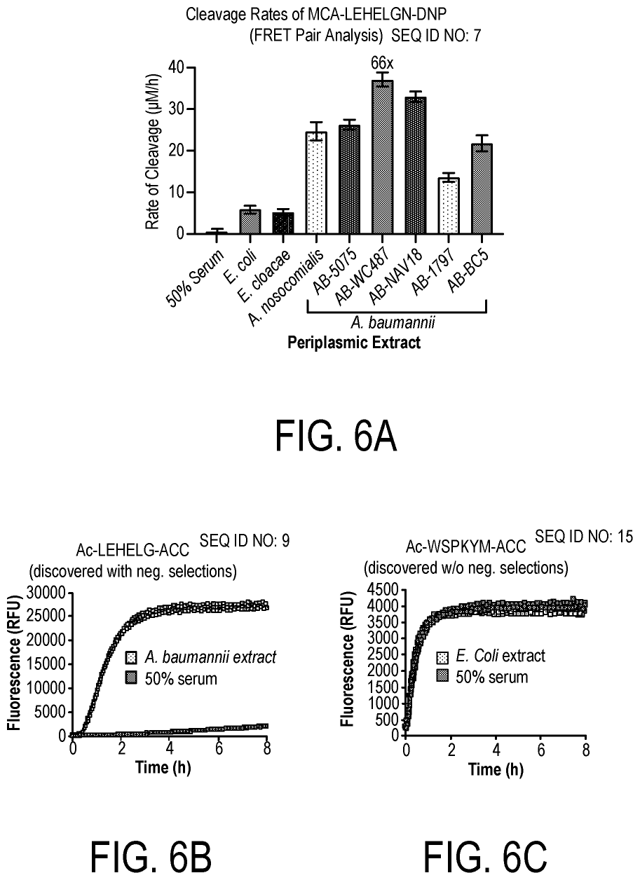

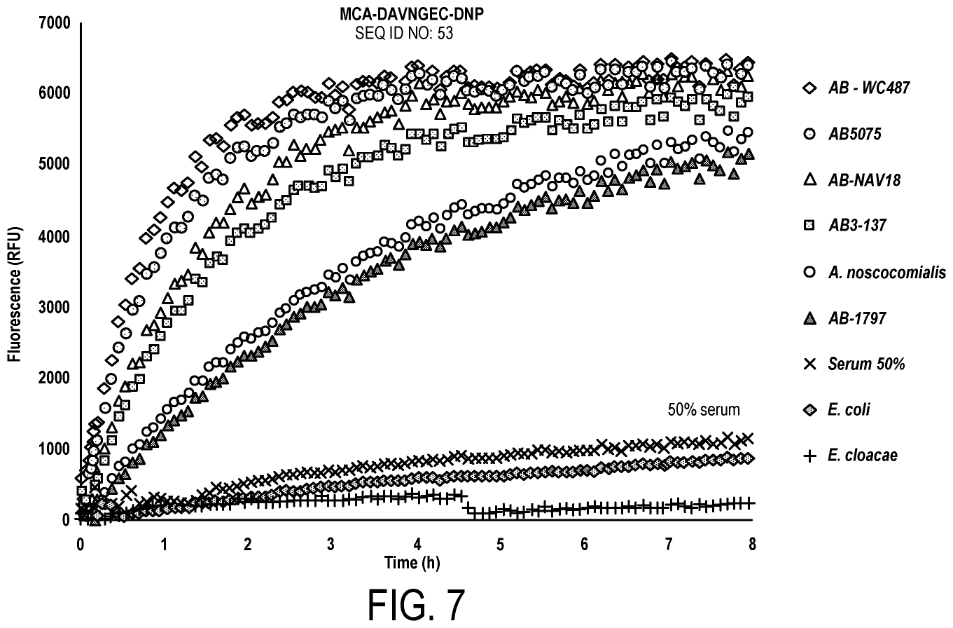

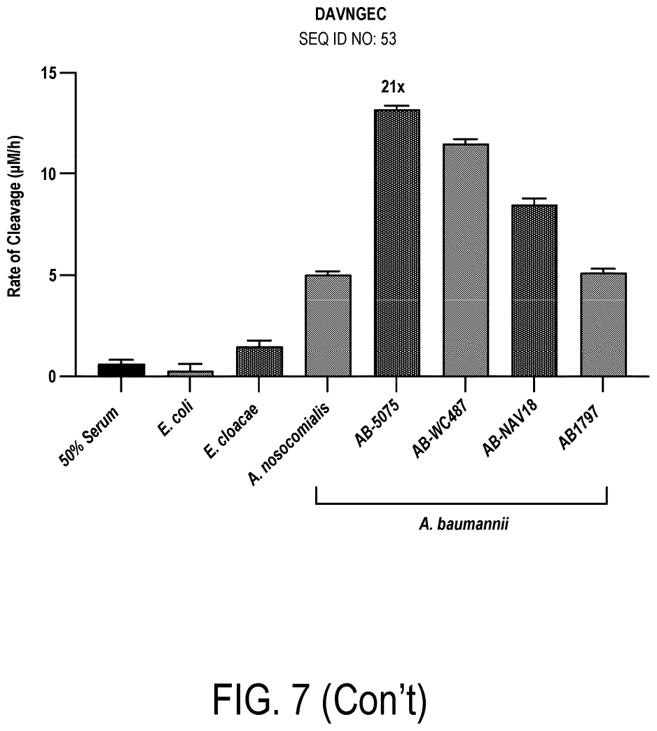

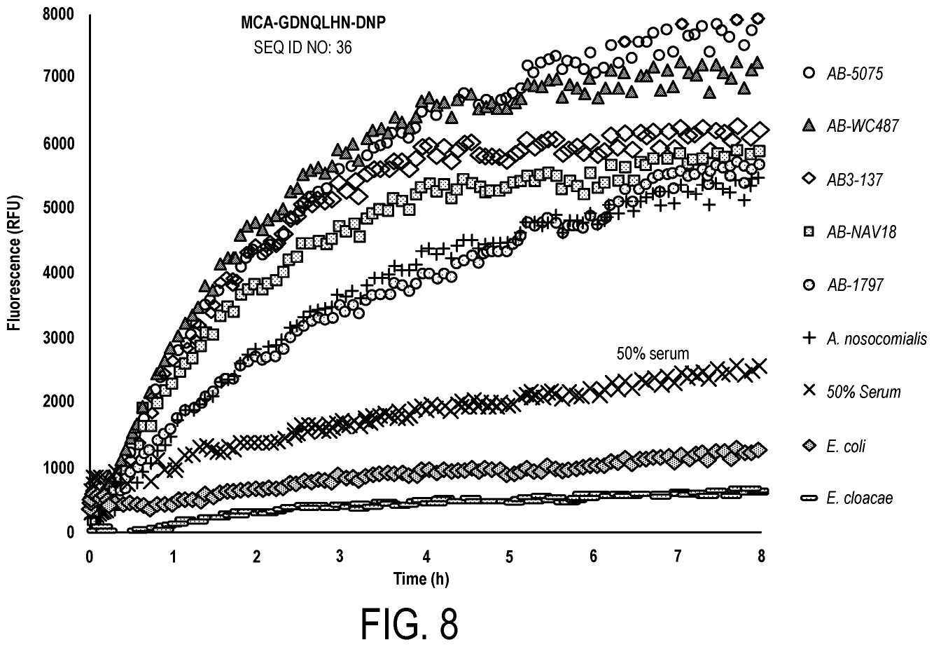

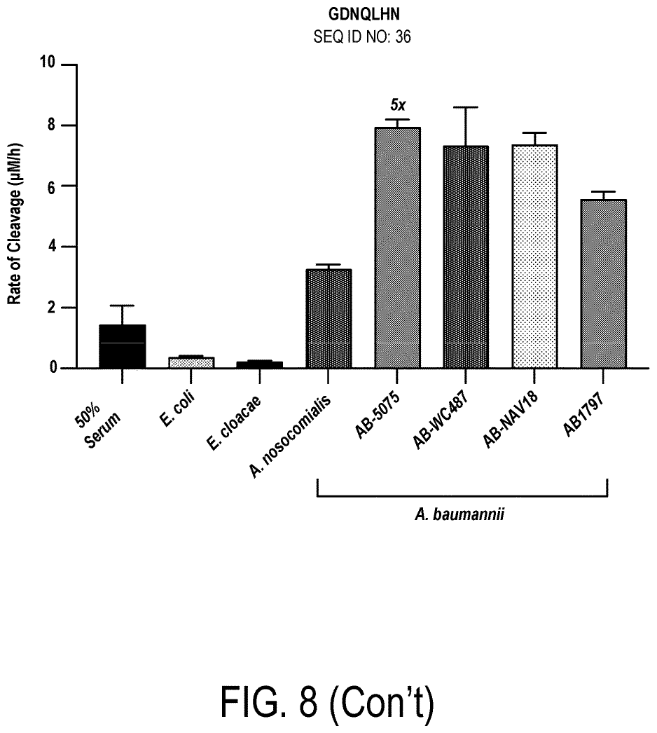

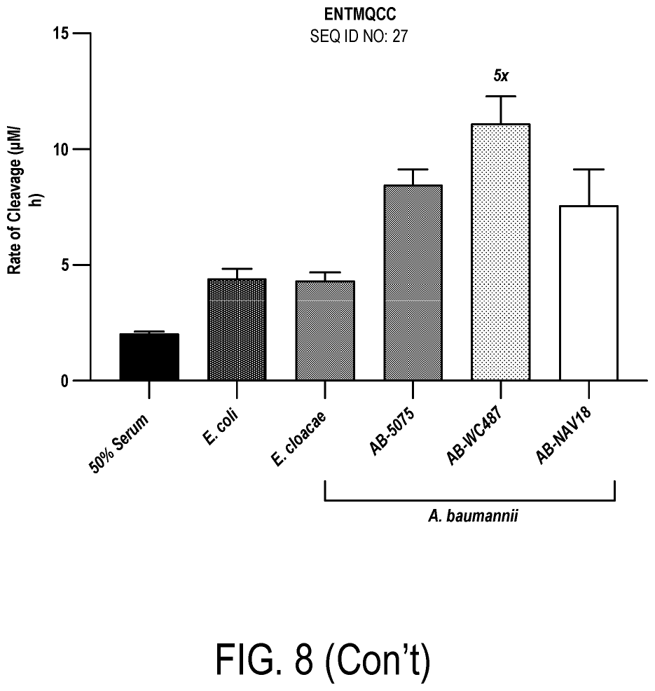

All FRET pairs were synthesized on solid phase as described in the Methods. Each FRET pair contained a dinitrophenyl (DNP) quencher at the C-terminus and a 7-methoxycoumarin-4-acetic acid (MCA) fluorophore at the N-terminus; fluorescence occurs when the linker is cleaved at any amino acid position (FIG. 4, step 3). 25 out of the 28 FRET pairs synthesized showed improved cleavage rates in A. baumannii periplasmic extract relative to serum (up to 66-fold, FIG. 5). Each symbol in FIG. 5 represents a peptide that is plotted as a ratio of its respective cleavage rates in periplasmic extract and serum [rate of cleavage in extract (μM/h)/rate of cleavage in serum (μM/h)]. The cleavage rates for one candidate are shown in FIG. 6A for eight Gram-negative periplasmic extracts and serum (also see FIGS. 7 and 8). Notably, given that A. baumannii extracts were used in positive selections, cleavage rates were much higher for A. baumannii and A. nosocomialis relative to non-Acinetobacter species (e.g. E. coli and E. cloacae), which speaks to the quality of the platform.

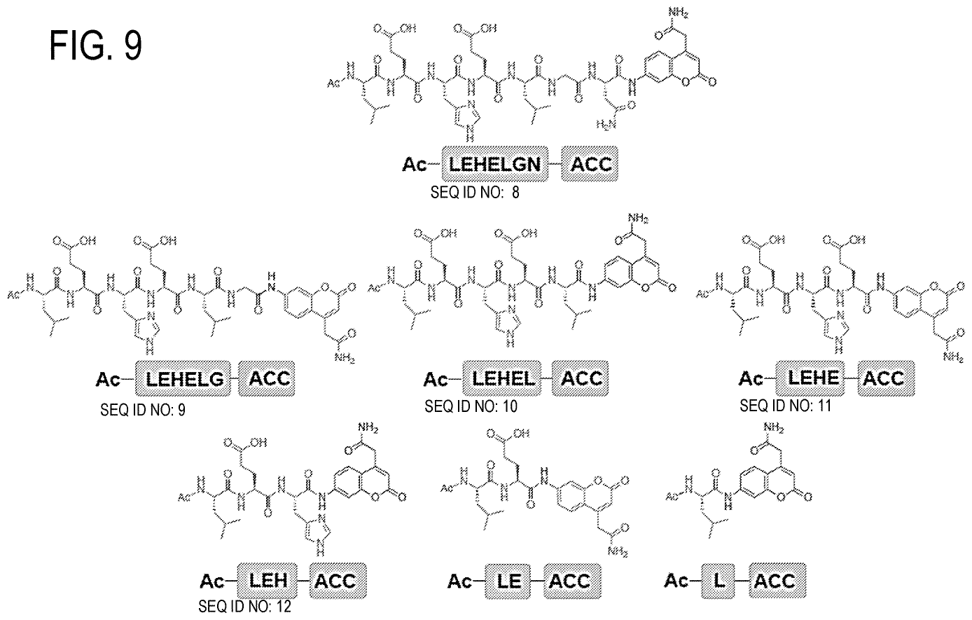

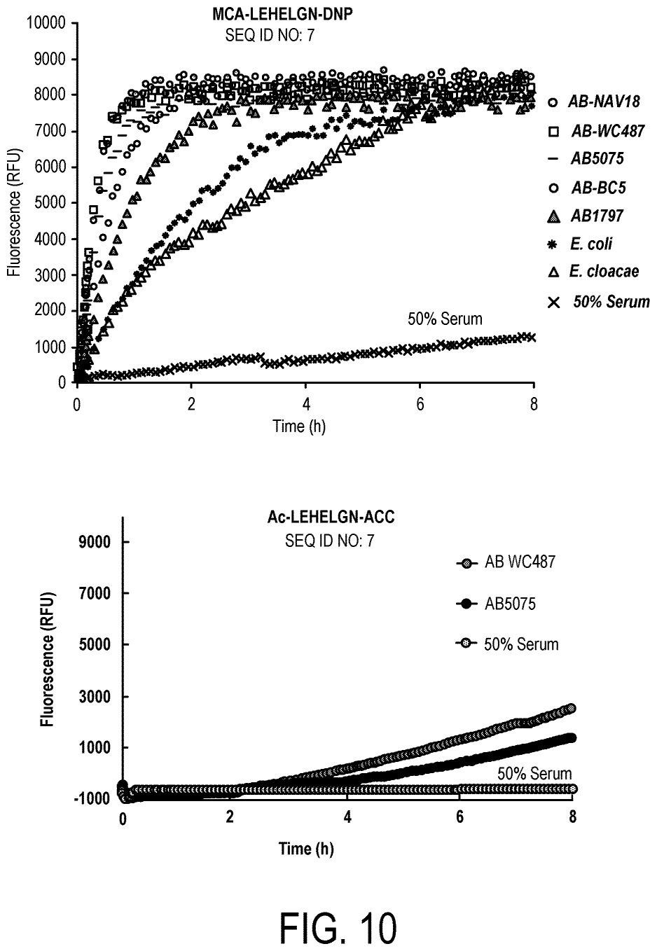

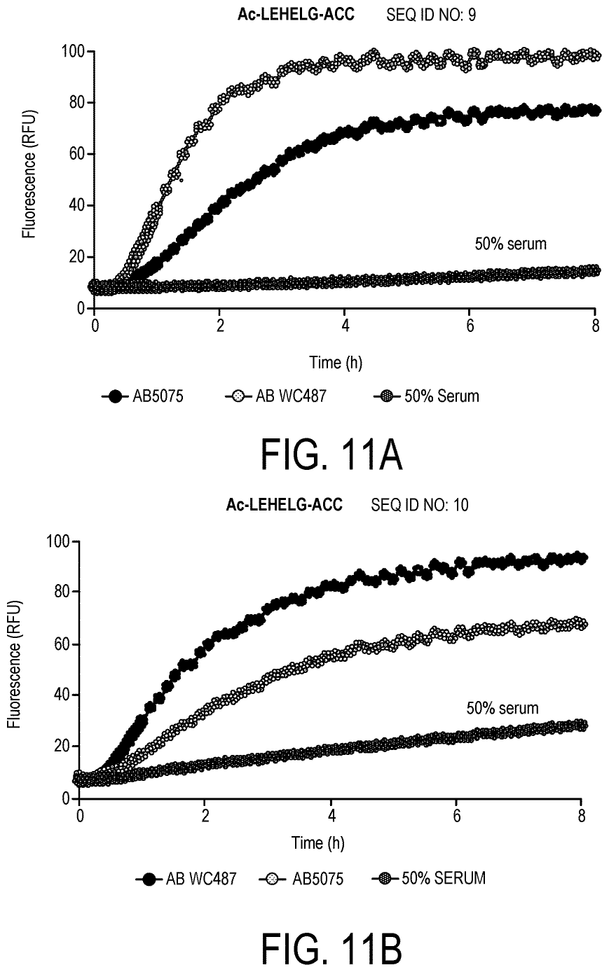

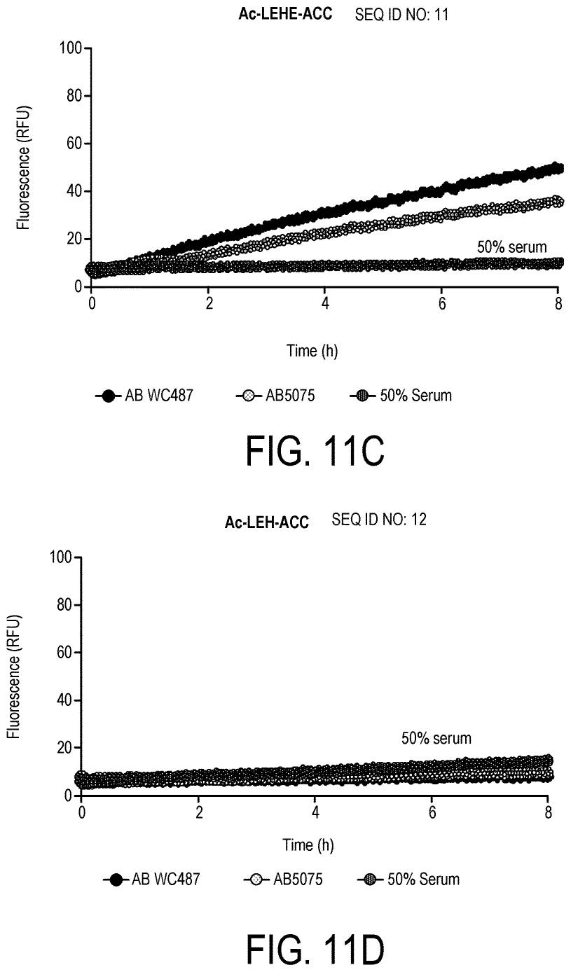

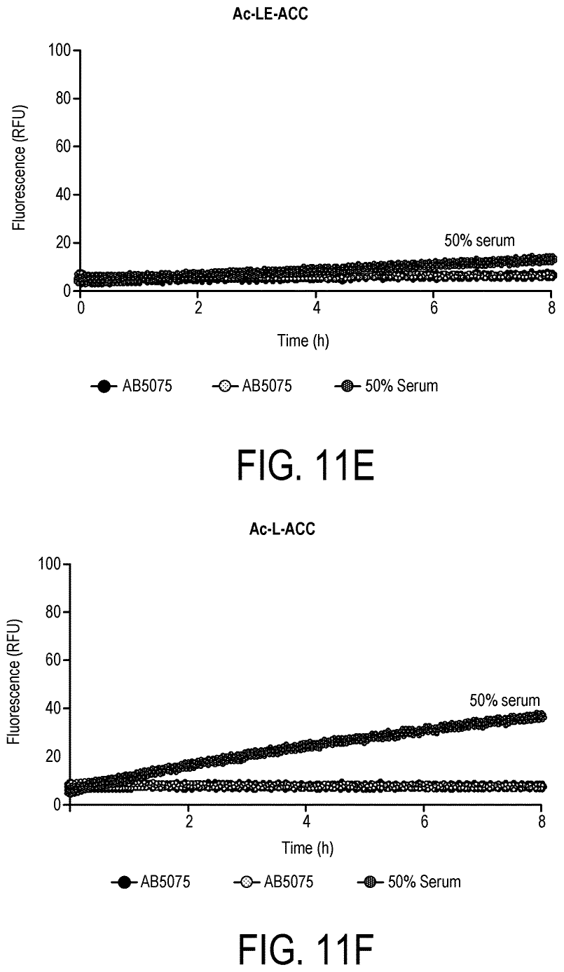

With the hit candidate in-hand, scarless cleavage was assessed with the turn-on fluorescent probe 7-amino-4-carbamoylmethylcoumarin (ACC), which only fluoresces when the ACC fluorophore is cleaved without an amino acid scar. To discover a scarless linker for LEHELGN (SEQ ID NO: 7), the following ACC conjugates (FIG. 9) were synthesized and cleavage rates were compared in 50% mouse serum and A. baumannii periplasmic extracts (FIGS. 10 and 11): Ac-LEHELGN-ACC (SEQ ID NO: 8), Ac-LEHELG-ACC (SEQ ID NO: 9), Ac-LEHEL-ACC (SEQ ID NO: 10), Ac-LEHE-ACC (SEQ ID NO: 11), Ac-LEH-ACC (SEQ ID NO: 12), Ac-LE-ACC, and Ac-L-ACC (note: Ac=acetyl). Minimal release of ACC from Ac-LEHELG-ACC (SEQ ID NO: 9) was observed over the course of 8 hours in serum while cleaving rapidly in A. baumannii periplasmic extract (FIG. 6B), which is in contrast to WSPKYM (SEQ ID NO: 14) that was discovered from SPD without negative selections (FIG. 6C) (20). Interestingly, all linkers containing the LEHE (SEQ ID NO: 13) component (FIGS. 11A-C) had improved serum stability relative to periplasmic extract.

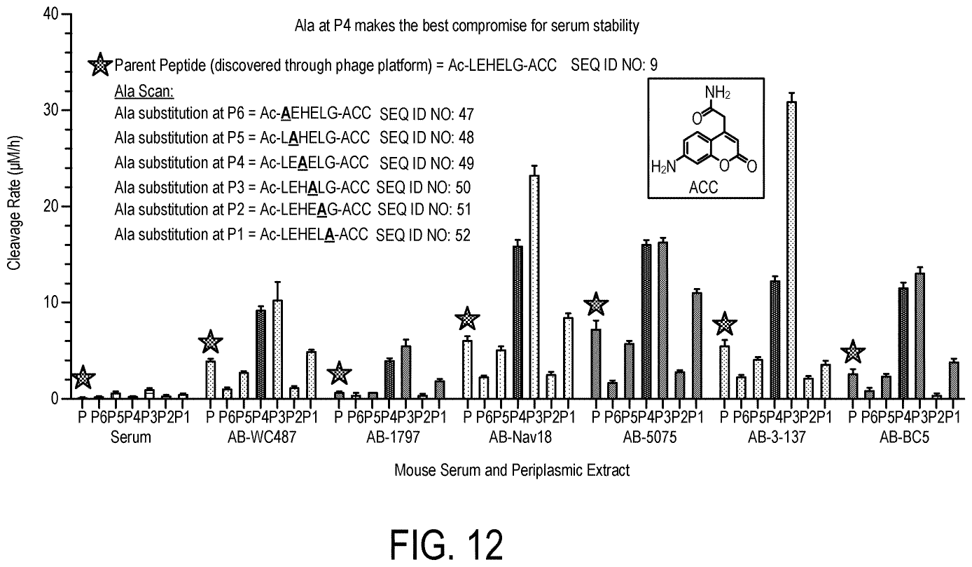

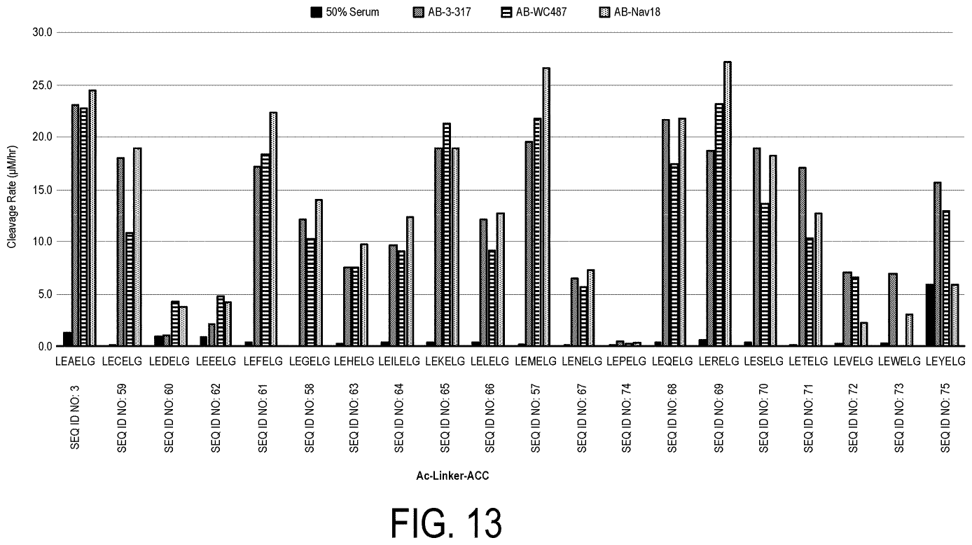

To determine how each amino acid contributed to the selectivity of linker LEHELG (SEQ ID NO: 1), an ala scan was conducted. Each position of the LEHELG (SEQ ID NO: 1) linker was substituted with a single alanine, the simplest amino acid containing a stereocenter, to create six variants. The P1 position is the amino acid directly adjacent to the cleavage site, which is conjugated to the ACC turn-on fluorophore. The cleavage rates of ala-substituted variants (P1-P6) were determined in mouse serum and in the periplasmic extracts from six strains of A. baumannii (FIG. 12). Importantly, the parent peptide showed the highest stability in mouse serum compared to all ala-substituted variants, which speaks to the platform's ability to discover peptide linkers that are stable in complex biological mixtures (e.g. serum). The data reveals that alanine substitutions at P3 and P4 provided the most substantial increase in cleavage rates relative to the parent peptide conjugate Ac-LEHELG-ACC (SEQ ID NO: 9). In contrast to the P3 variant, substitution of alanine at P4 (Ac-LEAELG-ACC (SEQ ID NO: 49)) did not show a significant loss of serum stability relative to the parent peptide but dramatically enhanced the cleavage rates in periplasmic extracts. Given this result, 19 additional natural amino acids were substituted at P4 and the cleavage rates of the resulting 19 ACC substrates were evaluated in A. baumannii periplasmic extract and serum (FIG. 13), which led to additional candidates of interest based on their selectivity profiles.

For full conjugate synthesis, the HMM siderophore (see FIG. 3) was chosen due to its ability to actively transport antibiotics into diverse bacteria. Solithromycin was chosen as a warhead to determine the quality of the cleavable linker in bacteria and to provide insight into the ability of the conjugate to actively transport through membranes. Solithromycin is a broad-spectrum antibiotic (G⁺ and GG⁻), and conjugates with this warhead require cleavage for activity, except in cases where the conjugate is actively transported to the cytoplasm, where the intact conjugate engages the target ribosome. When the minimum inhibitory concentration (MIC, minimum amount of drug to inhibit growth) of solithromycin equals the MIC of its cleavable L-linker conjugate, it indicates quantitative drug release, assuming the non-cleavable D-linker conjugate is not active (Table 1). D-linkers are not cleaved by most proteases. Proteolysis of the LEAELG conjugate led to activity in 9 strains, showing quantitative drug release since activity of the cleavable conjugate matched (within margin of error) or exceeded that of the attached drug (solithromycin), and the non-cleavable conjugate was inactive, with the exception of E. coli (Table 1).

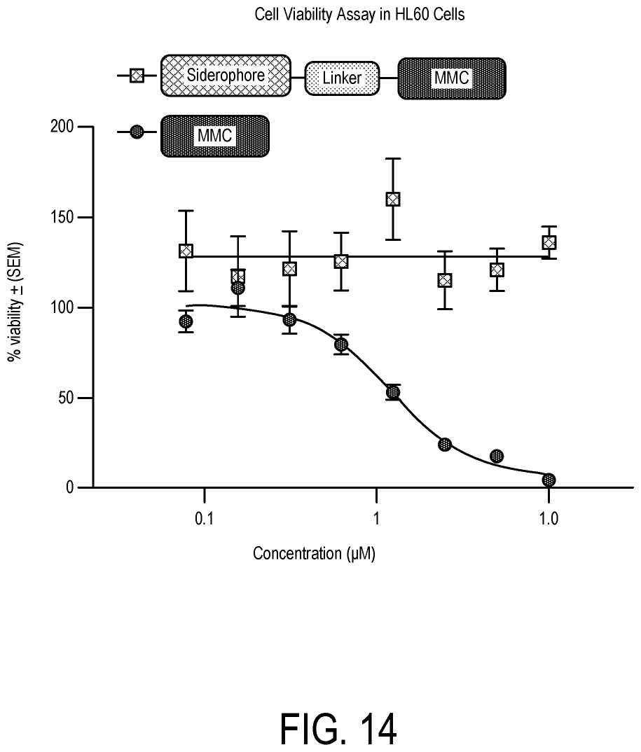

To convert cytotoxic molecules into non-toxic antibiotics through siderophore-mediated delivery, SCCs were synthesized through conjugation of an HMM siderophore to candidate linkers on solid phase (see Methods). Mitomycin C was chosen as a chemotherapeutic for conjugation to the C-terminus due to its potency in G⁺ and G⁻ pathogens. MICs were evaluated in G⁻ and G⁺ pathogens. Controls (e.g. conjugate without siderophore, conjugate without warhead, conjugate with non-cleavable D-linker) were synthesized to evaluate on-target activity and confirm mechanism of action. Importantly, minimal toxicity (EC₅₀>10 μM, FIG. 14) of HMM siderophore-LEHELG (SEQ ID NO: 1)-Mitomycin C was observed in HL60 cells relative to mitomycin C (EC₅₀ 1 μM, FIG. 14), along with activity in G⁻ pathogens (MIC 3-5 μM, P. aeruginosa, K. pneumoniae, E. aerogenes, and E. coli) and G⁺ pathogens (MIC<0.3 μM, S. aureus).

### Materials

All materials not described below were purchased from commercial suppliers and were of the highest grade available. Mouse serum was purchased in 1.0 or 5.0 mL quantities from MP Biochemicals. The enzyme BirA was expressed according to a known protocol. The HMM siderophore was synthesized by a known protocol.

### Pathogens and Cell Lines

Cell lines and bacteria strains were purchased from ATCC or acquired from BEI Resources.

### Reagents and Solvents

DCM, DMF, THF, diethyl ether, and acetonitrile to be used in anhydrous reaction mixtures were dried by passage through activated alumina columns immediately prior to use. Hexanes used were ≥85% n-hexane. Other commercial solvents and reagents were used as received, unless otherwise noted. Fmoc-protected amino acids were purchased from GL Biochem and Chem-Impex International. 2-(6-Chloro-1H-benzotriazole-1-yl)-1,1,3,3-tetramethylaminium hexafluorophosphate (HCTU), trifluoroacetic acid (TFA) were purchased from Chem-Impex International and Millipore Sigma, respectively. 4-Methylpiperidine was purchased from TCI. 2,4-Dinitrophenyl (DNP) NovaTag resin was purchased from Millipore Sigma. Fmoc-7-amino-4-carbamoylmethylcoumarin (ACC) Rink amide resin was purchased from Kimia Corporation. All other reagents and relevant catalysts were purchased from Millipore Sigma, Acros, Alfa Aesar, and Strem Chemicals.

### Instrumentation

All reactions were carried out in oven-dried glassware under an argon atmosphere unless otherwise noted. Analytical thin-layer chromatography (TLC) was performed using glass plates pre-coated with silica gel (0.25-mm, 60-Å pore size, 230-400 mesh, SILICYCLE INC) impregnated with a fluorescent indicator (254 nm). TLC plates were visualized by exposure to ultraviolet light (UV), and then were stained by submersion in a basic aqueous solution of potassium permanganate or with an acidic ethanolic solution of anisaldehyde, followed by brief heating. An Acquity UPLC BEH C18 1.7 μm column was used for analytical UPLC-MS. Preparative HPLC purification was performed on a Waters Prep HPLC or Varian ProStar HPLC using a C4 or C18 column. Analytical reverse-phase HPLC analyses were performed on an Agilent 1100 series HPLC system using a Phenomenex Kinetex 2.6 μm C18, 50×2.1 mm column. Flow rates were controlled at 0.35 mL/min. Peptide detection was based on UV absorption at 220 nm, while conjugate detection (following siderophore attachment) was based on UV absorption at 254 nm. Mitomycin conjugates were monitored at 220 nm, 254 nm, and 360 nm for HPLC purifications and UPLC-MS analysis. Mass spectra were obtained using a Waters Acquity UPLC system. Compound names, molecular formulas, and calculated exact masses for compounds were generated using the software built into CambridgeSoft-PerkinElmer's ChemDraw.

### Methods

### Periplasmic Extract Isolation:

Bacteria were grown from glycerol stocks on LB agar at 37 °C for 18 hours. Added a single colony to 1000 mL of LB base broth. The flask was incubated with shaking until an OD₆₀₀ of 0.5 was reached (~5-6 hours). The cultures were centrifuged at 12000 rpm for 10 minutes at 4 °C. The supernatant was poured into bleach, so as not to disturb the pelleted cells. Filter-sterilized 20% sucrose in 30 mM Tris (50 mL, pH 8) was added to the cell pellets. The pellets were gently suspended with a 50-mL serological pipet to achieve a homogenous suspension, and the contents were mixed on a shaker for 10 minutes on ice in a cold room at 4 °C. The suspension was then centrifuged at 4150 rpm for 20 minutes at 4 °C. The supernatant was decanted into bleach so as not to disturb the pellets. Filter-sterilized water (50 mL, prechilled to 4 °C) was added, and the pellet was gently suspended with a 50-mL serological pipet in a cold room. The homogenous suspension was allowed to shake for 10 minutes on ice in a cold room at 4 °C. The suspension was then centrifuged at 4150 rpm for 20 minutes at 4 °C. The supernatant (periplasmic extract) was filtered through a 500-mL vacuum filter (PES: 0.22 μm), ensuring that the filtrate remained at 4 °C by keeping the plastic filtration container on ice. The filtrate was then concentrated in an Amicon Ultra-15 Centrifugal Filter Ultracel-10 kDa (15-mL) by centrifuging at 4150 rpm for up to 15 minutes at 4 °C. The process was repeated until the periplasmic extract reached a volume of ~2-4 mL and a total protein concentration 1-5 mg/mL as indicated by a Nanodrop. For samples containing a higher total protein concentration than 5 mg/mL, the sample was diluted with Tris (50 mM, pH 8.0) to achieve a concentration ~5 mg/mL. The periplasmic extract was then aliquoted (200-400 μL/Eppendorf tube) into 1.5-mL Eppendorf tubes, flash-frozen with liquid nitrogen, and stored in a −80 °C freezer. [Note: To avoid reduction in protease activity, glycerol was not added to the periplasmic extract prior to freezing. All experiments were conducted with a fresh aliquot of periplasmic extract. Directly before use, the periplasmic extract was thawed over the course of 15 minutes in a 4 °C cold room.]

### Total Protein of Periplasmic Extracts and Mouse Serum:

(Measured Abs at 280 nm using a Nanodrop under the setting 1 Abs=1 mg/mL):
A. baumannii ATCC BAA-1797:1.13 mg/mL
A. baumannii BC-5:1.85 mg/mL
A. baumannii Nav-18:2.11 mg/mL
A. baumannii WC487: 5.19 mg/mL
A. baumannii 3-137:5.36 mg/mL
A. baumannii 5075UW: 5.28 mg/mL and 4.32 mg/mL
E. cloacae ATCC 13047:1.22 mg/mL
E. coli K12 MG1655: 1.46 mg/mL
Mouse Serum purchased from MP Biochemicals: >50 mg/mL

### Phage Library Construction:

All phages were propagated in the E. coli TG1 strain (Lucigen). E. coli CJ236 was purchased from Lucigen. Pierce streptavidin-coated high-capacity plates were purchased from ThermoFisher Scientific™. gBlocks and primers were purchased from IDT. The AviTag-displaying M13 phage libraries were constructed in the phagemid vector pCES1. After digesting the plasmid with ApaLl and Not1-HF at 37 °C for 3 hours. The backbone was isolated by DNA gel and extracted. Fusion of the gblock 5′-TTG TTC CTT TCT ATT CTC ACA GTG CAC TTA TGG GCC TGA ACG ATA TTT TTG AAG CGC AGA AAA TTG AAT GGC ATG AAG GCG GCA GCC TCG AGT TTG AAG CGC AGG GTG GTA GTG CGG CCG CAC ATC ATC ATC ACC-3′ (SEQ ID NO: 77) was accomplished by Gibson assembly affording the protein sequence as MGLNDIFEAQKIEWHEGGSLEFEAQGGSAAAHHHHHHGAAEQKLISEEDLNG-(SEQ ID NO: 78). The plasmid was electroporated into CJ236 cells. Single-stranded M13 DNA was purified using the M13 DNA kit from Qiagen. For library construction, the protocol by Chen and Sidhu was followed (85). Phage libraries were constructed using an oligonucleotide that encoded the reverse complement of the protein sequence-IEWHEGGSGGXXXXXXXGGSAAAHHH-(SEQ ID NO: 79) using the primer 5′-TGA TGA TGA TGT GCG GCC GCA CTA CCA CC (M: 50500000) (N: 25252525) (N) (M) (N) (N) (M) (N) (N) (M) (N) (N) (M) (N) (N) (M) (N) (N) (M) (N) (N) A CCA CCG CTG CCG CCT TCA TGC CAT TCA AT-3′ (SEQ ID NO: 80) for the AviTag-GGXXXXXXX-(SEQ ID NO: 81) library (codon M=A, C; N=A, C, T, G). Double-stranded DNA was generated according to Sidhu et al. (85, 86) and electroporated the CCC-dsDNA into E. coli TG1 using BioRad Gene Pulser Xcell Total System (1 cm cuvette, 1.8 kV field strength as a preset protocol, time constant: 4.1 seconds). The cells were rescued by adding 1 mL recovery media prewarmed to 37 °C. The media was transferred to a 15-mL culture tube and an additional 1 mL of recovery media was used to rinse the cuvette. The 2-mL culture was incubated for 1 hour at 37 °C with shaking at 200 rpm, and the culture was then plated on three 15-cm LB (AMP) agar plates (0.4 mL culture per plate). The plates were incubated for 15 hours at 37 °C. Added 4-5 mL 2×YT (AMP) media to lift the colonies from the plate. The library phagemid was isolated from the culture using a GeneJET Plasmid Miniprep Kit. The plasmid was incubated with Xh01 to remove the template from the library. XhoI digestion: Plasmid (20 μg) was digested with 120 U of XhoI enzyme in 200 μL solution for 2 hours. The digestion was then subjected to a PCR clean-up using a Qiagen Kit. The XhoI-cut phagemid was electroporated into TG-1 cells as described above and plated onto LB (AMP) agar plates. Following incubation at 37 °C for 15 hours, the colonies were lifted with 4-5 mL 2×YT (AMP) and transferred to 2×YT (AMP) [25-mL] to provide a culture with an OD of 0.1. The culture was incubated at 37 °C until an OD of 0.5 was achieved. Helper phage was then added to the culture in a 20:1 phage-to-cells ratio, and the culture was incubated at 37 °C without shaking for 25 minutes. The culture was then incubated at 37 °C with shaking for 25 minutes. The culture was centrifuged, the supernatant was removed, and the pellet was suspended in prewarmed 2×YT containing Kanamycin (25 μg/μL) and Ampicillin (100 μg/μL). The culture was incubated at 30 °C for 15 hours and centrifuged. The supernatant was collected and the phage precipitated with ⅕ volumes of PEG/NaCl solution (20% PEG 8000 w/v, 2.5 M NaCl) on ice for 15 minutes. A library size of 2.0×10⁹ cfu/mL was generated. The full protein sequence of the library: M-GLNDIFEAQKIEWHE-GGSGG-XXXXXXX-GGS-AAAHHHHHH-GAA EQKLISEEDLNGAA-gene 3 protein (g3p), where X is any amino acid.

### Biotinylation of Phage Library:

For phage biotinylation, purified phages (4×10¹² cfu) were resuspended in 10 mM Tris (0.5 mL, pH 8.0) and concentrated in a 30 kDa MWCO tube; this process was repeated three times to ensure buffer exchange. Then was added water (40 μL), 2× biotinylation buffer (80 μL, composition of biotinylation buffer: 0.1 M Tris, 10 mM MgCl₂, 2 mM Biotin, pH 8.0) to wash the 30 kDa MWCO tube and transfer the remaining solution to a 1.5-mL Eppendorf tube. Additional water (20 μL) and 2× biotinylation buffer (20 μL) was used to complete the transfer to the Eppendorf tube. Then was added 0.1 M ATP (~6 μL) and BirA enzyme (2 μL, 3 U/μL) to the phage solution (~200 μL). The mixture was vortexed and cooled to 4 °C overnight for biotinylation. An anti-biotin western blot was performed to confirm that phages were biotinylated.

### Substrate Phage Selection Process:

Periplasmic extracts from bacterial strains were isolated by osmotic shock from a variety of A. baumannii bacterial strains, including A. baumannii ATCC BAA-1797, A. baumannii WC487, A. baumannii 3-137, A. baumannii 5075UW, A. baumannii Nav-18, and A. baumannii BC5. Periplasmic extracts were diluted to 1-1.5 mg/mL for round 1 and concentrations were further diluted to 0.5-0.6 mg/mL for subsequent rounds. Serum was either used directly without dilution or as a 50% solution in Tris (50 mM, pH 8.0). Each periplasmic extract and serum was only allowed to undergo one freeze-thaw cycle to prevent degradation of protease activity. Phage samples contained 10¹²-1014 phage as determined by titer analysis. The diversity of the initial naïve library was confirmed by Next Generation Sequencing following amplification of the phagemid insert containing the randomized sequence (vide infra for PCR amplification conditions).

Step 1: Biopanning Procedure: High-binding capacity streptavidin 96-well plates (Pierce™, Thermo Fisher cat no: 15500) are blocked with 2% BSA for 30 minutes and washed with phosphate-buffered saline (PBS) containing 0.1% Tween 20 (PBST). Select wells were then coated with biotinylated M13 phage, with each well containing a maximum capacity of 10⁹ phage, by gently shaking the phage (100 μL) for 30 min. The wells were then washed with PBST and Tris at 37 °C. For each round of selection, mouse serum (50 L) was first incubated at 37 °C with shaking for 3 hours, the wells were then washed with PBST and Tris, followed by incubation with periplasmic extract (50 μL) at 37 °C with shaking for 30 minutes to 3 hours. The amount of incubation time for periplasmic extract was decreased with each subsequent round of selection, while the conditions for mouse serum remained constant from round to round. Following their incubation with the immobilized phage library, the serum and periplasmic extract were removed from the wells and added to TG-1 culture [15 mL, OD₆₀₀ 0.5; TG-1 culture was propagated in 2×YT media (without antibiotics) from a single colony grown on M9 minimal salts agar plates purchased from Teknova]. The two cultures were incubated at 37 °C without shaking for 25 minutes and subsequently with shaking for 25 minutes. The cultures were centrifuged, supernatant removed, and the pellet was resuspended in 2×YT media (1 mL) and plated on two 15-cm LB/AMP agar plates and allowed to incubate at 37 °C for 18 hours. Ampicillin resistant 2×YT media (20 mL) was added to each plate and the colonies were gently mixed on the agar surface and transferred to a 50-mL Falcon tube. An aliquot of the cultures was removed and miniprepped utilizing the GeneJET Plasmid Miniprep Kit to isolate the phagemid DNA. The cultures (100-200 μL, OD₆₀₀ 10) were then added to ampicillin resistant 2×YT media (20 mL) until an OD₆₀₀ of 0.1 is achieved. The cultures were incubated with shaking at 37 °C until OD₆₀₀ 0.5 is achieved (~45 minutes to 1.5 hours). Helper phage (10 μL, 2×10¹³ cfu/mL) was added to each culture. The cultures were then incubated at 37 °C without shaking for 25 minutes and with shaking for 25 minutes. The cultures were centrifuged for 10 minutes at 4150 rpm, the supernatant was removed, and the pellets resuspended in prewarmed 2×YT (25 mL, 25 μg/mL kanamycin and 100 μg/mL ampicillin). The cultures were incubated for 12 hours at 30 °C, centrifuged at 4150 rpm for 30 minutes or 15,000 rpm for 10 minutes, the supernatant was isolated, and the phage were precipitated by adding 5 mL of PEG/NaCl solution (20% PEG 8000 w/v, 2.5 M NaCl). The supernatant/PEG salt solution mixture was allowed to sit on ice for 15-30 minutes before centrifuging to obtain a white pellet of M13 phage containing the amplified sequences of interest. The phage were resuspended in PBS (1-2 mL, pH 7.4) and buffer exchanged into 10 mM Tris (pH 8) in a 5 mL Amicon filter tube (30 kDa). To the concentrated phage suspension (~80 μL) in a 1.5-mL Eppendorf tube was then added 2× biotinylation buffer (40 μL, containing 0.1M Tris, 10 mM MgCl₂, 2 mM biotin), Tris (60 μL, 0.1 M), freshly prepared adenosine triphosphate (ATP, 0.1 M, 20 μL), and BirA enzyme (20 μL, ~5 mg/mL). The reaction was allowed to mix on a rotator for 8 hours at room temperature. To the reaction was then added PBS (600 μL, pH 7.4), PEG salt solution (20% PEG 8000 w/v, 2.5 M NaCl), and the mixture was allowed to sit on ice for 15 minutes. The Eppendorf tubes containing the mixture were then centrifuged at 14,000 rpm, the supernatant was removed, and the pellet was resuspended in 0.1 M Tris (400 μL, pH 8) followed by the addition of PEG salt buffer (100 μL). The process of precipitation was repeated 3× to remove excess biotin from the reaction mixture, after which the pellet was resuspended in PBS (50-150 μL depending on the size of the phage pellet, pH 7.4). The phage pellets were diluted with 2% BSA in PBST to a final volume of 300 μL. This sample was then subjected to the next round of selection.

Step 2: PCR Amplification and Barcoding: For each sample, the isolated phagemid DNA (2 μL, ~5 ng/μL) prepared as indicated above using a GeneJET Plasmid Miniprep Kit) was subjected to PCR amplification to amplify the insert containing the randomized portion of the library. The following reverse and forward primers (2 μL, 10 μM) were added to the phagemid respectively: GAGTTCAGACGTGTGCTCTTCCACACTCTTTCCCATGGTGATGATGATGTGCGG (SEQ ID NO: 82) and AATGATACGGCGACCACCGAGATCTACACGCAGAAATTGAATGGCATGA (SEQ ID NO: 83). Kapa HiFi fidelity buffer (5×, 10 μL), dNTPs (1 μL, 10 mM), H₂O (32 μL), and KAPA HiFi polymerase (1 μL) were then added to the reaction mixture in a 0.2-mL PCR tube. PCR amplification proceeded under the following conditions in sequence: 1) 95 °C for 3 minutes, 2) 98 °C for 20 seconds, 3) 65 °C for 15 seconds, 4) 72 °C for 15 seconds, 5) 72 °C for 1 minutes, 6) repeat steps 2-4 for 20 cycles, 7) 4 °C. The product from the PCR reaction was purified by DNA gel and the relevant band was excised and the DNA was extracted using a GeneJET Gel Extraction Kit. The isolated phagemid DNA was barcoded. The barcoding PCR involved the following components: plasmid (2 μL, 5 ng/μL), Kapa HiFi fidelity buffer (5×, 10 μL), forward barcoding primer AATGATACGGCGACCACCGAGATCTACACGCAGAAAATTGAATGGCATGA (SEQ ID NO: 84) (5 μL, 0.1 M), dNTPs (1 μL, 10 mM), H₂O (25 μL), True-Seq-barcode-primer CAAGCAGAAGACGGCATACGAGATXXXXXXXGTGACTGGAGTTCAGACGTGTGCT CTTCC (SEQ ID NO: 85) (5 μL, 0.1 μM, note: XXXXXXX (SEQ ID NO: 86) represents the reverse compliment of the unique barcode used; a total of 47 unique barcodes were utilized in these experiments), and KAPA HiFi Polymerase (1 μL). The reaction mixture was subjected to the following PCR protocol: 1) 94 °C for 1 minute, 2) 94 °C for 30 seconds, 3) 60 °C for 30 seconds, 4) 72 °C for 1 minute, 5) repeat steps 2-4, 6) 94 °C for 10 minutes (at this stage, added 1 μL of a 10 μM hot start primer mix containing the following primers at this stage: AATGATACGGCGACCACC (SEQ ID NO: 87) and CAAGCAGAAGACGGCATAC (SEQ ID NO: 88); primers were obtained commercially from IDT and used as 100 μM stock solutions), 7) 94 °C for 30 seconds, 8) 55 °C for 30 seconds, 9) 72 °C for 1 minute, 10) repeat steps 7-9 twelve times, 11) 72 °C for 5 minutes. The PCR product was diluted with water to a total volume of 100 μL and purified with the GeneJET PCR Purification Kit (note: isopropanol was used to improve the DNA yield). A DNA gel was run on all samples to confirm the purity of each product. Any samples that contained more than one band were purified by DNA gel extraction as described above. From these experiments, a total of 150 samples were barcoded, which included cleavage data from each positive (periplasmic extract) and negative (mouse serum) selection conducted for each of up to nine rounds of selection, along with biological replicates. The concentration of each sample was adjusted to 3 ng/μL (23.7 nM) as determined using the Quant-iT Qubit dsDNA HS Assay Kit and a Qubit 4 fluorometer. Each phagemid sample were combined into four LowBind 1.5-mL Eppendorf tubes in 10 μL quantities (~38 samples per tube). A DNA gel was run on each combination to reveal a single band. The custom Read1 primer for NGS sequencing is GACCACCGAGATCTACACGCAGAAAATTGAATGGCATGA (SEQ ID NO: 89) (100 μM, 10 μL), which was added to a separate LowBind 1.5-mL Eppendorf tube. The library construct size for each sample was 192 base pairs. The samples were analyzed by HiSeq_SE50. All samples contained the following sequence, with the randomized region denoted by X:

(SEQ ID NO: 90)
AATGATACGGCGACCACCGAGATCTACACGCAGAAAATTGAATGGCATGA
AGGCGGCAGCGGTGGTXXXXXXXXXGGTGGTAGTGCGGCCGCACATCATC
ATCACCATGGGAAAGAGTGTGGAAGAGCACACGTCTGAACTCCAGTCACX
XXXXXXATCTCGTATGCCGTCTTCTGCTTG.

Step 3: NGS and Data Analysis: The DNA sequences resulting from NGS analysis contained 92% high quality reads. Each sample contained 5-10 million reads, and only high quality reads were included in the analysis. Conversion to amino acids and comparison of each positive selection (periplasmic extract cleavages) to its corresponding negative selection (mouse serum cleavages) was achieved using two Python scripts. Comparison of data across multiple rounds of selection was achieved by a third Python script. Scoring of hit candidates for follow-up synthesis and evaluation of cleavage kinetics was achieved with a fourth Python script. The workflow for data analysis was conducted in three steps: 1) The positive/negative read count was compared for each sequence in each round of selection, which enables the identification of sequences that were found more frequently in positive selections than in negative selections for each individual round; 2) The pos/negative read counts for sequences were compared across multiple rounds of selection to identify sequences that consistently cleaved more frequently in positive selections over negative selections; 3) Select sequences are scored and ranked based on their frequency of reads in positive relative to negative selections across the entire data set. The extent of sequence enrichment in subsequent rounds of selection relative to the initial naïve library was also considered when prioritizing sequences for synthesis and evaluation of cleavage kinetics. A total of thirty sequences were prioritized for evaluation. The final Python script for data analysis incorporated a scoring platform, for which the criteria is summarized as follows: step 1) any sequence that has a positive read count greater than or equal to 100 and a positive/negative read count greater than or equal to 3 would be counted in category 1 for any round of selection, step 2) any sequence that has a positive/negative read count less than 3 but greater than or equal to 1.5 would be counted in category 2, step 3) any sequence that has a positive/negative read count between 1 and 1.5 would be counted in category 3, step 4) any sequence that has a negative/positive read count between 1 and 1.5 would be counted in category 4, step 5) any sequence that has a negative/positive read count greater than 1.5 could be counted in category 5, step 6) any sequence that has a positive read count less than 5 and a negative round count less than 5 would be considered insignificant and not included in the analysis. Sequences that were found primarily in categories 1 and 2, with minimal occurrences in category 5 across all rounds of selection were prioritized. The data pointed to 705 sequences of interest. Any sequences in the list that contained arginine or lysine were eliminated. In addition to the scoring criteria described above, one sequence (SADLNNQ, SEQ ID NO: 40) that was highly enriched in rounds 6, 7, 8, and 9 was also prioritized, which was independent of the scoring platform described. A total of 28 sequences were selected for synthesis and evaluation. A total of 25 sequences showed improved cleavage rates in periplasmic extract relative to serum. Three sequences were false positives.

### Fluorescence Assays:

### Cleavage of MCA-Linker-DNP (FRET Pairs) and Ac-Linker-ACC Conjugates in Periplasmic Extract and Serum:

Periplasmic extracts and mouse serum were not diluted prior to use in fluorescence assays and were allowed to undergo only one freeze-thaw cycle prior to use. The total protein for different periplasmic extracts used in these experiments are listed above. Solutions for substrate (40 μM, 2×) were prepared in Tris (pH 8.0) from 2 mg/mL stock solutions in DMSO. Each 40 μM solution (25 μL) was then transferred to a 96-well plate (opaque, 200 μL/well round bottom) in triplicate. Flash frozen periplasmic extract and commercial mouse serum were slowly thawed in a cold room at 4 °C over 15 min. Periplasmic extract (25 μL, Total Protein: 1-5 mg/mL as listed above) or mouse serum (25 μL, Total Protein: >50 mg/mL) was transferred in triplicate to wells containing substrate in a cold room at 4 °C. The plate was sealed with a transparent polyolefin silicone film (Thermofisher™ Nuc™ Sealing Tape, 12-565-513) and placed in a plate reader at 37 °C with shaking at 225 rpm for 8 hours. MCA excitation maximum: 325. MCA emission maximum: 392 nm. ACC excitation maximum: 355 nm. ACC emission maximum is 460 nm.

### Minimum Inhibitory Concentration (MIC) Assays by the Broth Microdilution Method:

Broth and Agar Preparation: Mueller-Hinton II (MH-II, cation-adjusted) broth was prepared from solid MH-II (22 g) and MilliQ H₂O (1 L), which was autoclaved in a presterilized 1-L glass bottle. The autoclaved MH-II media contained dipyridyl (DP, 200 μM) for MIC assays involving siderophore conjugates. MH-II agar plates were prepared from autoclaved MH-II (22 g), Bacto agar (17 g), and MilliQ water (1 L). The bacterial strains were obtained from glycerol stocks.

Bacteria Culture Preparation: Bacteria were streaked on MH-II agar plates from glycerol stocks. The streaked plates were placed in an incubator at 37 °C without shaking for 18-hours. A sterilized pipet tip was gently touched on the surface of a single colony and added to autoclaved MH-II broth (5 mL) in a sterilized 14-mL culture tube. The cultures were incubated for 18 hours at 37 °C with shaking at 215 rpm. The cultures were diluted 200-fold into autoclaved MH-II broth (4 mL) and incubated at 37 °C with shaking at 215 rpm for 3-5 hours, or until an OD 600 of 0.5 was achieved. An aliquot of the culture (1 mL) was transferred to a sterilized culture tube and diluted with filter-sterilized PBS (~7 mL) until an OD₆₀₀ of 0.13-0.15 was achieved. An aliquot (11.5 μL-10 μL) of the cultures was then transferred into MH-II media (15-mL) to achieve a concentration of 1×10⁵ cells/mL. The diluted cultures (90 μL/well) were then added to substrate (10 μL at the following concentrations: 640 μg/mL, 320 μg/mL, 160 μg/mL, 80 μg/mL, 40 μg/mL, 20 μg/mL, 10 μg/mL, and 0 μg/mL in sterilized 96-well plates to achieve a final substrate concentration of 64 μg/mL, 32 μg/mL, 16 μg/mL, 8 μg/mL, 4 μg/mL, 2 μg/mL, 1 μg/mL, and 0 μg/mL. The 96-well plates were then sealed with sterilized gas-permeable covers. The plates were incubated at 37 °C with shaking at 215 rpm for 16 hours. Each well condition was prepared in triplicate, and each MIC assay was repeated on a different day. The turbidity of each well was examined visually and the MIC was counted as the lowest concentration that lacked turbidity.

Cell Culture: HL-60 cells were cultured at 37 °C in a 5% CO₂ atmosphere. The cells were grown in Iscove's Modified Dulbecco's Medium, supplemented with 20% fetal bovine serum (FBS) and 0.1% penicillin-streptomycin. Media was replaced every other day until cells reached the desired confluency.

Viability Assay: HL-60 cells (4×10\hat{ }3) were seeded in a 96-well plate in cell culture media (100 μL) for 24 hours at the atmosphere stated above (5% CO₂). After incubation, the cells were treated with 10 μL of vehicle or drug (0.07-10 μM, n=6) for 24 hours at 37 °C. Cell viability was assessed using MTT. MTT media (30 μL, 5 mg/mL) was added to each well for 2 hours and plate was centrifuged at 500×g for 5 minutes after, followed by media aspiration. The formazan crystals left were solubilized in DMSO and absorbance (540 nm) was measured using a microplate reader (SpectraMax M5). Data obtained was normalized to the vehicle to generate final values. The graph plotted in FIG. 14 is depicted as the mean±SEM vs. compound concentration.

Solid-phase peptide synthesis (SPPS): Peptides are synthesized on 0.025-0.30 mmol scale using a peptide synthesizer (Biotage peptide synthesizer, Syro II Parallel peptide synthesizer, and CEM Liberty Blue 2.0 peptide synthesizer). 2-Chlorotrityl resin (Chem-Impex, 1.0-2.0 mmol/g) was used for siderophore-peptide-drug conjugates, DNP NovaTag resin (Novabiochem, 0.30-0.60 mmol/g) was used for FRET pair synthesis, and 7-Amino-4-carbamoylmethylcoumarin (ACC) Rink amide AM resin (Kimia Corp., 0.15 mmol/g) was used for ACC conjugate synthesis. Resin was either swelled in DCM at room temperature for 5 minutes or in DMF at 70 °C for 20 minutes. Fmoc deprotection was carried-out with 20% 4-methylpiperidine in DMF. Each amino acid (5.0 equiv, 0.5 M in DMF) was attached via two sequential coupling steps with HCTU (5.0 equiv, 0.5 M in DMF) and DIEA (10 equiv, 0.5 M in DMF). At least three washes were conducted after Fmoc deprotection and coupling steps. Resin cleavage from 2-chlorotrityl resin was accomplished with AcOH/TFE/DCM (1:1:3) to provide peptide without removal of acid-sensitive protecting groups. Resin cleavage and global deprotection from DNP NovaTag resin and ACC Rink amide resin was accompolished with a cocktail of EDT (50 μL), H₂O (50 μL), TIPS (25 μL), and TFA (1.88 mL).

### General Procedure for the Synthesis of FRET Pair Conjugates (Compounds 1A-25A):

Step 1: Preparation of NH₂-Linker-DNP resin. A Biotage peptide synthesizer was used to couple amino acids P1-P7 (15 equiv, 500 mM) to DNP NovaTag resin (63 mg, 0.025 mmol) with HCTU (15 equiv, 500 mM) and DIEA (30 equiv, 500 mM) using microwave-assisted synthesis (55 °C, 5 minutes). Deprotections with 20% 4-methylpiperidine were conducted at 75 °C (3 minutes). Swelling of the starting P1-conjugated Dnp NovaTag™ resin (Novabiochem, 0.30-0.60 mmol/g loading) was achieved in DMF (70 °C, 20 minutes). Reaction cycle includes Fmoc deprotection, washing, coupling, and post-coupling washing steps. After the final Fmoc deprotection of the last amino acid, the resin is washed with DMF (3×5 mL), MeOH (3×5 mL), and DCM (3×5 mL). The resin is then dried under reduced pressure.

Step 2: Synthesis of MCA-Linker-DNP-resin: Attachment of MCA fluorophore to N-terminus. The starting resin was swelled in DCM (1×1.5 mL) prior to use. To a 10-mL Biotage peptide synthesis polypropylene fritted reactor vial was added NH₂-Linker-DNP resin (0.025 mmol) from Step 1, MCA (0.75 mL, 15 equiv, 500 mM in NMP), HCTU (0.75 mL, 15 equiv, 500 mM in NMP), and DIEA (1.5 mL, 30 equiv, 500 mM in NMP). The reaction vessel was protected from light and shaken for 3 hours using a microplate shaker fitted with a test tube rack. The reaction was filtered upon completion. The resin was washed with NMP (3×6 mL), MeOH (2×4 mL), and DCM (3×4 mL). The coupling procedure was repeated an additional two times.

Step 3: Synthesis of MCA-Linker-DNP conjugates: Resin cleavage and global deprotection.

To the MCA-Linker-DNP resin from Step 2 in a 10-mL reaction peptide synthesis vessel was added deprotection cocktail [EDT (50 μL), H₂O (50 μL), TIPS (25 μL), and TFA (1.88 mL)]. The vessel was shaken for 30 minutes using a microplate shaker fitted with a test tube rack. Upon completion, the reaction mixture was filtered into a 20-mL scintillation vial and concentrated under reduced pressure. The residue was azeotroped with benzene (3×3 mL) and washed with Et₂O (3×3 mL). The Et₂O washes were filtered through a cotton plug and discarded. The remaining solid residue was dissolved in 80% DMSO/H₂O and purified by HPLC (5 to 70% MeCN in H₂O with 0.1% TFA, 50 minutes) to provide the MCA-Linker-DNP conjugates as bright yellow solids.

### General Procedure for the Synthesis of ACC Conjugates (Compounds 1B-31B):

Step 1: Preparation of Fmoc-amino acid-ACC resin. The ACC conjugates (ref1: Harris, J. L.; Backes, B. J.; Leonetti, F.; Mahrus, S.; Ellman, J. A.; Craik, C. S. Proc. Natl. Acad. Sci. U.S.A 2000, 97, 7754. ref2: Maly, D. J.; Leonetti, F.; Backes, B. J.; Dauber, D. S.; Harris, J. L.; Craik, C. S.; Ellman, J. A. J. Org. Chem., 2002, 67, 910) are prepared from ACC-rink amide AM resin (Fmoc-ACC resin) by the following general procedure: To a 50-mL glass solid-phase peptide synthesis vessel containing Fmoc-ACC resin (300 mg, 0.045 mmol) was added DMF (15 mL), and the resin was swelled by mixing for 15 min. Fmoc deprotection was achieved by mixing 4-methylpiperidine (10 mL, 40% in DMF) with the ACC resin for 3 min, followed by mixing 4-methylpiperidine (10 mL, 20% in DMF) for 10 min. The resin was then washed with DMF (4×6 mL), with mixing for 3 minutes each. A freshly prepared stock solution of Fmoc-amino acid-OH (1.44 mL, 500 mM in DMF), HATU (1.44 mL, 500 mM in DMF), and 2,4,6-collidine (1.20 mL, 600 mM in DMF) were added to the ACC resin. The resin was then mixed for 12 h. The process was repeated three times. After loading the first amino acid (P1), the resin was capped with a 1.2 M solution of Ac₂O in DMF (15 mL), which was mixed with the resin for 15 min. The resin was then washed with DMF (4×6 mL).

Step 2: Preparation of Fmoc-Linker-ACC resin. The Fmoc-amino acid-ACC resin (0.045 mmol) prepared from Step 1 was then transferred to a 10-mL Biotage reaction vessel, and the remainder of the amino acids were coupled on a Biotage peptide synthesizer. Deprotections were accomplished with 20% 4-methylpiperidine at 75 °C (3 minutes). Amino acid couplings were accomplished with Fmoc-amino acid-OH (15 equiv, 500 mM), HCTU (15 equiv, 500 mM), and DIEA (30 equiv, 500 mM) using microwave-assisted synthesis (55 °C, 5 minutes). Swelling was achieved in DMF (70 °C, 20 minutes). Reaction cycle includes Fmoc deprotection, washing, coupling, and post-coupling washing steps. After the final Fmoc deprotection of the last amino acid, the resin is washed with DMF (3×5 mL), MeOH (3×5 mL), and DCM (3×5 mL). The resin is then dried under reduced pressure.

Step 3: Preparation of Ac-Linker-ACC resin: Fmoc deprotection and acetylation.

The Fmoc-Linker-ACC resin from Step 2 was Fmoc deprotected by mixing 40% 4-methylpiperidine (10 mL) for 3 min, followed by mixing 20% 4-methylpiperidine for 10 minutes. The resin was then washed with DMF (4×6 mL), with mixing for 3 minutes each. Acetylation of the N-terminus was accomplished by mixing the resin with DMF (11 mL), DIEA (2.3 mL), and Ac₂O (2.3 mL) for 15 min. The resin was then filtered and washed with DMF (4×6 mL), MeOH (2×4 mL), and DCM (2×4 mL).

Step 4: Preparation of Ac-Linker-ACC: Resin cleavage and global deprotection. To the Ac-Linker-ACC resin from Step 3 in a 10-mL reaction peptide synthesis vessel was added deprotection cocktail [EDT (50 μL), H₂O (50 μL), TIPS (25 μL), and TFA (1.88 mL)]. The vessel was shaken for 30 minutes using a microplate shaker fitted with a test tube rack. Upon completion, the reaction mixture was filtered into a 20-mL scintillation vial and concentrated under reduced pressure. The residue was azeotroped with benzene (3×3 mL) and washed with Et₂O (3×3 mL). The Et₂O washes were filtered through a cotton plug and discarded. The remaining solid residue was dissolved in 80% DMSO/H₂O and purified by HPLC (5 to 50% MeCN in H₂O with 0.1% TFA, 45 minutes) to provide the Ac-Linker-ACC conjugates as white solids.

### Preparation of HMM Siderophore-LEHELG-Solithromycin Tri-TFA Salt:

Step 1: Preparation of Fmoc-glycine-2-chlorotrityl resin: To a 500-mL flame-dried, round-bottom flask was added dried Fmoc-glycine (446 mg, 1.5 mmol, predried on high vacuum for 12 hours). The flask was purged with argon, and the amino acid was then suspended in anhydrous DCM (20 mL). The flask was sonicated for 5 minutes to assist with solubilizing the amino acid. Dry 2-chlorotrityl resin (Chem-Impex, 2.00 g, 3.0 mmol, 1.0-2.0 mmol/g, predried on high vacuum for 12 hours) was added to the amino acid suspension in one portion, and the sides of the flask were rinsed with additional anhydrous DCM (10 mL). DIEA (0.6 mL, 3.45 mmol) was then added, and the mixture was mixed vigorously on a Mistral Multi-Shaker® for 24 h. The resin was then filtered through a 50-mL solid phase peptide synthesis vessel and washed with DCM (3×20 mL). Resin capping was achieved by adding a 2.5% solution of MeOH (0.4 mL) and DIEA (0.4 mL) in DCM (15.2 mL), and the solid phase peptide synthesis vessel was rotated for 30 minutes on a nutator. The resin was then washed with DCM (3×20 mL) and dried on high vacuum to provide 2.37 g (1.79 mmol, 0.755 mmol/g, 60% yield) of Fmoc-glycine-2-chlorotrityl resin.

Step 2: Preparation of Fmoc-LEHELG-2-chlorotrityl resin: To a fritted SPPS reaction vessel containing Fmoc-Gly-2-chlorotrityl resin (1.78 g, 0.537 mmol/g loading) was added 20% 4-methylpiperidine in DMF (50 mL), followed by nitrogen mixing for 20 minutes. After removing the deprotection solution, the resin was washed with DMF (4×100 mL) with mixing for 5 minutes each. This process was repeated. To the Fmoc-deprotected material was added a solution of Fmoc-amino acid-OH (11.5 mL, 500 mM in DMF), a solution of HATU (11.4 mL, 500 mM in DMF), and a solution of DIEA (22.9 mL, 500 mM in DMF). The contents were mixed for 45 minutes with nitrogen at room temperature. The coupling solution was removed, and the resin was washed with DMF (2×100 mL). The deprotection and coupling steps were repeated for the remaining amino acids. The final resin was washed with DMF (3×100 mL), MeOH (2×100 mL), and DCM (3×100 mL), and dried under reduced pressure to provide Fmoc-LEHELG-2-chlorotrityl resin (1.79 g, 0.686 mmol, 0.383 mmol/g loading, 72%).

Step 3: Preparation of LEHELG-2-chlorotrityl resin: To the Fmoc-LEHELG-2-chlorotrityl resin (407.0 mg, 0.16 mmol 0.383 mmol/g) isolated from Step 2 was added 20% 4-methylpiperidine, followed by mixing for 10 minutes (2×6 mL) on a nutator. After each Fmoc-deprotection, the resin was washed with DMF (4×6 mL, mixed for 3 minutes/wash) and DCM (3×6 mL, allowed to sit for 3 minutes/wash without mixing). The material was dried under reduced pressure to provide LEHELG-2-chlorotrityl resin (351.0 mg, 0.134 mmol, ~0.383 mmol/g, 86%).

Step 4: Preparation of Protected-HMM Siderophore-LEHELG-OH: To a fritted 10-mL Biotage peptide synthesis vessel containing LEHELG-2-chlorotrityl resin (149.0 mg, 0.057 mmol 0.383 mmol/g) from Step 3 was added a solution of protected HMM siderophore (183 μL, 500 mM in DMF, 1.6 equiv) (Boyce et al. J Am Chem Soc. 2020, 142 (51), 21310-21321), HCTU (180 μL, 500 mM in DMF, 1.58 equiv), and DIEA (365 μL, 500 mM in DMF, 3.2 equiv). The reaction mixture was mixed by shaking for 6 hours. The coupling solution was drained, and the resin was washed sequentially with DMF (3×6 mL), DCM (3×6 mL), MeOH (3×6 mL), and DCM (3×6 mL). Then was added a solution of 2:2:6 AcOH/TFE/DCM (2 mL). The reaction mixture was shaken for 12 minutes. The reaction mixture was filtered into a 20-mL scintillation vial and the resin was rinsed with DCM (3×3 mL). The combined filtrate was concentrated under reduced pressure. The crude residue was sonicated and azeotropped with benzene (3×5 mL) to provide protected-HMM siderophore-LEHELG-OH (73.6 mg, 72%) without the need for further purification.

Exact mass calculated for C103H114N10018: 1778.83, ESI LC/MS: [M+H]=1779.6

Step 5: Preparation of Protected-HMM Siderophore-LEHELG-Solithromycin bis-TFA salt: To a 5-mL flame-dried, round-bottom flask was added the protected-HMM siderophore-LEHELG-OH (26.9 mg, 15.1 μmol) from Step 4, solithromycin (25.0 mg, 29.6 μmol, 1.96 equiv), HOBt (20% water, 5.79 mg, 30.2 μmol, 2.0 equiv), and EDC (4.35 mg, 22.7 μmol, 1.5 equiv). The flask was purged with argon and anhydrous DMF (0.15 mL, 0.1 M) was added, followed by anhydrous DIEA (11 μL, 60.4 μmol, 4.0 equiv). After stirring at room temperature for 4 hours, DMSO (8 mL) and water (0.5 mL) was added. The solution was filtered and purified by prep HPLC (5 to 95% MeCN in water with 0.1% TFA, 40 minutes). The fractions containing the desired product were combined and lyophilized to provide protected-HMM siderophore-LEHELG-solithromycin bis-TFA salt (20.4 mg, 48%) as a white powder.

Exact mass calculated for C146H177FN16027: 2605.30, ESI LC/MS: [M+H]=2606.3

Step 6: Preparation of HMM Siderophore-LEHELG-Solithromycin tri-TFA salt (Compound 3C): To a 20-mL scintillation vial containing protected-HMM siderophore-L-LEHELG-solithromycin (18.5 mg, 6.5 μmol) from Step 5 was added a solution of TFA/TIPS-H/water/EDT (1.88 mL, 0.025 mL, 0.05 mL, 0.05 mL). The reaction mixture was allowed to stir for 30 minutes at room temperature. The reaction mixture was concentrated under reduced pressure and azeotroped with benzene (3×3 mL) with sonication between azeotropes. The solid residue was washed with Et₂O (3×5 mL) and filtered through a cotton plug. The crude solid was dissolved in DMSO: H₂O (1:1, 4 mL) and purified by HPLC (5-80% MeCN in H₂O with 0.1% TFA) to provide HMM siderophore-LEHELG-solithromycin tri-TFA salt (3C, 9.7 mg, 60%) as a white powder after lyophilization.

Exact mass calculated for C93H131FN16O27: 1922.94, ESI LCMS: [M+H]=1923.8

### Preparation of HMM Siderophore-LEHELG-Mitomycin C Formate Salt (Compound 11C):

Step 1. Preparation of HMM siderophore-LEHELG-OPNP ester: To a flame-dried round bottom flask was added protected-HMM siderophore-LEHELG-OH (38.6 mg, 21.7 μmol). The flask was purged with argon, followed by the addition of anhydrous MeCN (1.10 mL, 0.02 M) and triethylamine (6.7 μL, 47.7 μmol, 2.2 equiv) forming a suspension. The reaction mixture was cooled to 0 °C and allowed to stir for 10 minutes at this temperature. p-Nitrophenyl chloroformate (9.6 mg, 47.7 μmol, 2.2 equiv) was then added. After stirring for 5 min, 4-dimethylaminopyridine (0.50 mg, 4.3 μmol, 0.2 equiv) was added and the reaction mixture was allowed to stir for 5 minutes at 0 °C. The cooling bath was removed, and the reaction mixture was allowed to warm to room temperature and stirred for 15 minutes. DMF (0.10 mL) was added dropwise over 10 min, resulting in a clear solution. Upon completion of the reaction as monitored by TLC (5% MeOH in DCM), the reaction was concentrated under reduced pressure and purified by silica gel chromatography (0 to 3% MeOH in DCM) to provide protected-HMM siderophore-LEHELG-OPNP ester (30.9 mg, 16.3 μmol, 75%) as an off-white residue. After transferring the product to a 20-mL scintillation vial, a solution of EDT (50 μL), H₂O (50 μL), TIPS (25 μL), and TFA (1.88 mL) was added. The reaction mixture was stirred for 30 minutes at room temperature under a nitrogen atmosphere. The reaction mixture was then concentrated under reduced pressure, the residue azeotroped with benzene (3×3 mL), and triturated with Et₂O. Et₂O was filtered through a cotton plug, and the filtered solid was dissolved in 75% DMSO in water (4 mL) and purified by HPLC (5 to 65% MeCN in water with 0.1% TFA, 90 minutes). The fractions containing desired product were collected and lyophilized to provide HMM siderophore-LEHELG-OPNP (18.7 mg, 86%) as a white solid.

Exact mass calculated for C56H71N11O20: 1217.49, ESI LCMS: [M+H]=1218.4

Step 2. Preparation of HMM Siderophore-LEHELG-Mitomycin C formate salt (Compound 11C): To HMM siderophore-LEHELG-OPNP (6.3 mg, 4.7 μmol) from Step 1 was added MMC (8.1 mg, 24 μmol, 5.1 equiv). The reaction flask was purged with argon and anhydrous DMF (0.48 mL) was added. The reaction mixture was stirred for 30 minutes at room temperature. Triethylamine (7.9 μL, 0.6 M in DMF, 1.0 equiv) was then added over the course of 3 minutes. The reaction mixture was allowed to stir for 45 minutes at room temperature. Another equivalent of triethylamine (7.9 μL, 0.6 M in DMF, 1.0 equiv) was then added, and the reaction was allowed to stir for an additional 30 minutes. The reaction mixture was concentrated under reduced pressure, and the residue was washed sequentially with 1% AcOH in MeCN (1×3 mL), MeCN (3×3 mL), 75% MeCN in NH₄OAc (aq, 10 mM). The 75% MeCN in aqueous NH₄OAc washes were combined and purified by prep HPLC (5 to 47% MeCN in H₂O with 0.1% formic acid, 90 minutes) to provide HMM siderophore-LEHELG-Mitomycin C formate salt (11C, 5.9 mg, 85%) was obtained as a white solid.

Exact mass calculated for C65H84N14O22: 1412.59, ESI LCMS: [M+H]=1413.3

### ABBREVIATIONS

Ac: acetyl
ACC: 7-amino-4-carbamoylmethylcoumarin
Ac2O: acetic anhydride
AcOH: acetic acid
BSA: bovine serum albumin
MeCN: acetonitrile
DCM: dichloromethane
DP: 2,2′-dipyridyl
DIEA: N,N-diisopropylethylamine
DMF: dimethylformamide
DNP: dinitrophenyl
EDC: 1-ethyl-3-(3-dimethylaminopropyl) carbodiimide
Et₂O: diethyl ether
EDT: ethanedithiol
Fmoc: fluorenylmethoxycarbonyl
Fmoc-ACC resin: fluorenylmethoxycarbonyl-7-aminocoumarin-4-acetic acid-rink amide AM resin
HCTU: O-(1H-6-chlorobenzotriazole-1-yl)-1,1,3,3-tetramethyluronium hexafluorophosphate
HATU: hexafluorophosphate azabenzotriazole tetramethyl uronium
HOBT: hydroxybenzotriazole
H₂O: water
hr: hour(s)
Kan: kanamycin
LB broth: Luria-Bertani broth
min: minute(s)
MeOH: methanol
MH-II: Mueller-Hinton II
MCA: 7-methoxycoumarin-4-acetic acid
MMC: mitomycin C
NaCl: sodium chloride
NGS: Next Generation Sequencing
NH4OAc: ammonium acetate
PBS: phosphate buffered saline
OPNP: p-nitrophenyl ester
rt: room temperature
SPPS: solid phase peptide synthesis
SCC: siderophore-chemotherapeutic conjugates
TIPS-H: triisopropylsilane
THF: Tetrahydrofuran
TFA: trifluoroacetic acid
2×YT: 2× Yeast Extract Tryptone

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

1. B. Ren et al., Tumor microenvironment participates in metastasis of pancreatic cancer. Mol. Cancer 17, 108 (2018).
2. L. Rahib, M. R. Wehner, L. M. Matrisian, K. T. Nead, Estimated Projection of US Cancer Incidence and Death to 2040. JAMA Netw Open 4, e214708 (2021).
3 M. Slodkowski et al., Current Approaches for the Curative-Intent Surgical Treatment of Pancreatic Ductal Adenocarcinoma. Cancers (Basel) 15 (2023).
4. K. Almhanna et al., A phase II study of antibody-drug conjugate, TAK-264 (MLN0264) in previously treated patients with advanced or metastatic pancreatic adenocarcinoma expressing guanylyl cyclase C. Invest. New Drugs 35, 634-641 (2017).
5. K. C. Park, M. Dharmasivam, D. R. Richardson, The Role of Extracellular Proteases in Tumor Progression and the Development of Innovative Metal Ion Chelators that Inhibit their Activity. Int J Mol Sci 21 (2020).
6. A. Lin et al., Off-target toxicity is a common mechanism of action of cancer drugs undergoing clinical trials. Sci Transl Med 11 (2019).
7. M. Alas, A. Saghaeidehkordi, K. Kaur, Peptide-Drug Conjugates with Different Linkers for Cancer Therapy. J. Med. Chem. 64, 216-232 (2021).
8. H. Zhao et al., A Potential Mechanism for ADC-Induced Neutropenia: Role of Neutrophils in Their Own Demise. Mol. Cancer Ther. 16, 1866-1876 (2017).
9. M. Dorywalska et al., Molecular Basis of Valine-Citrulline-PABC Linker Instability in Site-Specific ADCs and Its Mitigation by Linker Design. Mol. Cancer Ther. 15, 958-970 (2016).
10. A. Beck, L. Goetsch, C. Dumontet, N. Corvaïa, Strategies and challenges for the next generation of antibody-drug conjugates. Nat Rev Drug Discov 16, 315-337 (2017).
11. P. D. Senter, E. L. Sievers, The discovery and development of brentuximab vedotin for use in relapsed Hodgkin lymphoma and systemic anaplastic large cell lymphoma. Nat. Biotechnol. 30, 631-637 (2012).
12. N. G. Caculitan et al., Cathepsin B Is Dispensable for Cellular Processing of Cathepsin B-Cleavable Antibody-Drug Conjugates. Cancer Res. 77, 7027-7037 (2017).
13. J. T. W. Tong, P. W. R. Harris, M. A. Brimble, I. Kavianinia, An Insight into FDA Approved Antibody-Drug Conjugates for Cancer Therapy. Molecules 26 (2021).
14. V. P. Chavda, H. K. Solanki, M. Davidson, V. Apostolopoulos, J. Bojarska, Peptide-Drug Conjugates: A New Hope for Cancer Management. Molecules 27 (2022).
15. M. Poreba, Protease-activated prodrugs: strategies, challenges, and future directions. FEBS J. 287, 1936-1969 (2020).
16. X. S. Puente, L. M. Sanchez, C. M. Overall, C. Lopez-Otin, Human and mouse proteases: a comparative genomic approach. Nat. Rev. Genet. 4, 544-558 (2003).
17. N. D. Rawlings et al., The MEROPS database of proteolytic enzymes, their substrates and inhibitors in 2017 and a comparison with peptidases in the PANTHER database. Nucleic Acids Res. 46, D624-D632 (2018).
18. D. Matthews, J. Wells, Substrate phage: selection of protease substrates by monovalent phage display. Science 260, 1113-1117 (1993).
19. M. Whitney et al., Parallel in vivo and in vitro selection using phage display identifies protease-dependent tumor-targeting peptides. J. Biol. Chem. 285, 22532-22541 (2010).
20. J. H. Boyce et al., Platform to Discover Protease-Activated Antibiotics and Application to Siderophore-Antibiotic Conjugates. J. Am. Chem. Soc. 142, 21310-21321 (2020).
21. A. J. O'Donoghue et al., Global identification of peptidase specificity by multiplex substrate profiling. Nature methods 9, 1095-1100 (2012).
22. Y. Choe et al., Substrate Profiling of Cysteine Proteases Using a Combinatorial Peptide Library Identifies Functionally Unique Specificities. J. Biol. Chem. 281, 12824-12832 (2006).
23. M. Poreba, M. Drag, Current Strategies for Probing Substrate Specificity of Proteases. Curr. Med. Chem. 17, 3968-3995 (2012).
24. J. Zhou et al., Deep profiling of protease substrate specificity enabled by dual random and scanned human proteome substrate phage libraries. Proc. Natl. Acad. Sci. U.S.A 117, 25464-25475 (2020).
25. M. Feng, Y. Pan, R. Kong, S. Shu, Therapy of Primary Liver Cancer. Innovation (Camb) 1, 100032 (2020).
26. S. Louafi et al., Gemcitabine plus oxaliplatin (GEMOX) in patients with advanced hepatocellular carcinoma (HCC): results of a phase II study. Cancer 109, 1384-1390 (2007).
27. M. Garancini et al., Combined hepatocellular-cholangiocarcinoma: a population-level analysis of an uncommon primary liver tumor. Liver Transpl. 20, 952-959 (2014).
28. X. Cai et al., Background progenitor activation is associated with recurrence after hepatectomy of combined hepatocellular-cholangiocarcinoma. Hepatology 56, 1804-1816 (2012).
29. P. Song et al., Patients' prognosis of intrahepatic cholangiocarcinoma and combined hepatocellular-cholangiocarcinoma after resection. Cancer Med 8, 5862-5871 (2019).
30. E. De Martin et al., Analysis of Liver Resection Versus Liver Transplantation on Outcome of Small Intrahepatic Cholangiocarcinoma and Combined Hepatocellular-Cholangiocarcinoma in the Setting of Cirrhosis. Liver Transpl. 26, 785-798 (2020).
31. C. D. Malone et al., Outcomes of Yttrium-90 Radioembolization for Unresectable Combined Biphenotypic Hepatocellular-Cholangiocarcinoma. J. Vasc. Interv. Radiol. 31, 701-709 (2020).
32. M. Garland, J. J. Yim, M. Bogyo, A Bright Future for Precision Medicine: Advances in Fluorescent Chemical Probe Design and Their Clinical Application. Cell Chem Biol 23, 122-136 (2016).
33. M. Pazgan-Simon, S. Serafinska, J. Janocha-Litwin, K. Simon, J. Zuwala-Jagiello, Diagnostic challenges in primary hepatocellular carcinoma: case reports and review of the literature. Case Rep Oncol Med 2015, 878763 (2015).
34. T. Patel, Cholangiocarcinoma—controversies and challenges. Nat Rev Gastroenterol Hepatol 8, 189-200 (2011).
35. G. K. Abou-Alfa et al., Assessment of Treatment With Sorafenib Plus Doxorubicin vs Sorafenib Alone in Patients With Advanced Hepatocellular Carcinoma: Phase 3 CALGB 80802 Randomized Clinical Trial. JAMA Oncol 5, 1582-1588 (2019).
36. L. Liu, Y. H. Zheng, L. Han, S. K. Qin, Efficacy and safety of the oxaliplatin-based chemotherapy in the treatment of advanced primary hepatocellular carcinoma: A meta-analysis of prospective studies. Medicine (Baltimore) 95, e4993 (2016).
37. M. Vizovisek, D. Ristanovic, S. Menghini, M. G. Christiansen, S. Schuerle, The Tumor Proteolytic Landscape: A Challenging Frontier in Cancer Diagnosis and Therapy. Int J Mol Sci 22 (2021).
38. P. Zagami, L. A. Carey, Triple negative breast cancer: Pitfalls and progress. npj Breast Cancer 8, 95 (2022).
39. J. Shi, F. Liu, Y. Song, Progress: Targeted Therapy, Immunotherapy, and New Chemotherapy Strategies in Advanced Triple-Negative Breast Cancer. Cancer Management and Research 12, 9375-9387 (2020).
40. P. G. Prasanna et al., Therapy-Induced Senescence: Opportunities to Improve Anticancer Therapy. JNCI: Journal of the National Cancer Institute 113, 1285-1298 (2021).
41. H. Qayoom, N. A. Wani, B. Alshehri, M. A. Mir, An insight into the cancer stem cell survival pathways involved in chemoresistance in triple-negative breast cancer. Future Oncology 17, 4185-4206 (2021).
42. A. R. Schreiber, M. Andress, J. R. Diamond, Tackling metastatic triple-negative breast cancer with sacituzumab govitecan. Expert Rev. Anticancer Ther. 21, 1303-1311 (2021).
43. D. Nandini, A. Jennifer, D. Pradip, Therapeutic Strategies for Metastatic Triple-Negative Breast Cancers: From Negative to Positive. Pharmaceuticals (Basel) 14 (2021).
44. K. H. Negash, J. K. S. Norris, J. T. Hodgkinson, Siderophore-Antibiotic Conjugate Design: New Drugs for Bad Bugs? Molecules 24, 3314 (2019).
45. M. Ghosh et al., Siderophore Conjugates of Daptomycin are Potent Inhibitors of Carbapenem Resistant Strains of Acinetobacter baumannii. ACS Infect. Dis. 4, 1529-1535 (2018).
46. M. Ghosh et al., Targeted Antibiotic Delivery: Selective Siderophore Conjugation with Daptomycin Confers Potent Activity against Multidrug Resistant Acinetobacter baumannii Both in Vitro and in Vivo. J. Med. Chem. 60, 4577-4583 (2017).
47. R. Liu et al., A Synthetic Dual Drug Sideromycin Induces Gram-Negative Bacteria To Commit Suicide with a Gram-Positive Antibiotic. J. Med. Chem. 61, 3845-3854 (2018).
48. M. Miethke, M. A. Marahiel, Siderophore-based iron acquisition and pathogen control. Microbiol Mol Biol Rev 71, 413-451 (2007).
49. B. C. Chu et al., Siderophore uptake in bacteria and the battle for iron with the host; a bird's eye view. Biometals 23, 601-611 (2010).
50. L. Ma, A. Terwilliger, A. W. Maresso, Iron and zinc exploitation during bacterial pathogenesis. Metallomics 7, 1541-1554 (2015)
51. A. J. M. Messenger, R. Barclay, Bacteria, iron and pathogenicity. Biochemical Education 11, 54-63 (1983).
52. A. Pramanik et al., Albomycin is an effective antibiotic, as exemplified with Yersinia enterocolitica and Streptococcus pneumoniae. Int. J. Med. Microbiol. 297, 459-469 (2007).
53. T. A. Wencewicz, M. J. Miller, “Sideromycins as Pathogen-Targeted Antibiotics” in Topics in Medicinal Chemistry, J. Fisher, S. Mobashery, M. Miller, Eds. (Springer International Publishing, 2017), 10.1007/7355_2017_19 chap. Antibacterials, pp. 151-183.
54. M. G. P. Page, The Role of Iron and Siderophores in Infection, and the Development of Siderophore Antibiotics. Clin Infect Dis 69, S529-S537 (2019).
55. I. J. Schalk, Siderophore-antibiotic conjugates: exploiting iron uptake to deliver drugs into bacteria. Clin. Microbiol. Infect. 24, 801-802 (2018).
56. I. J. Schalk, L. Guillon, Fate of ferrisiderophores after import across bacterial outer membranes: different iron release strategies are observed in the cytoplasm or periplasm depending on the siderophore pathways. Amino Acids 44, 1267-1277 (2013).
57. I. J. Schalk, G. L. A. Mislin, K. Brillet, Structure, Function and Binding Selectivity and Stereoselectivity of Siderophore-Iron Outer Membrane Transporters. Curr. Top. Membr. 69, 37-66 (2012).
58. J. D. Faraldo-Gómez, M. S. P. Sansom, Acquisition of siderophores in Gram-negative bacteria. Nat. Rev. Mol. Cell Biol. 4, 105-116 (2003).
59. G. Tonziello, E. Caraffa, B. Pinchera, G. Granata, N. Petrosillo, Present and future of siderophore-based therapeutic and diagnostic approaches in infectious diseases. Infect Dis Rep 11, 8208-8208 (2019).
60. A. Paulen, F. Hoegy, B. Roche, I. J. Schalk, G. L. A. Mislin, Synthesis of conjugates between oxazolidinone antibiotics and a pyochelin analogue. Bioorg. Med. Chem. Lett. 27, 4867-4870 (2017).
61. A. Paulen et al., Synthesis and antibiotic activity of oxazolidinone-catechol conjugates against Pseudomonas aeruginosa. Org. Biomol. Chem. 13, 11567-11579 (2015).
62. C. Hennard et al., Synthesis and Activities of Pyoverdin-Quinolone Adducts: A Prospective Approach to a Specific Therapy AgainstPseudomonas aeruginosa. J. Med. Chem. 44, 2139-2151 (2001).
63. F. Rivault et al., Synthesis of pyochelin-norfloxacin conjugates. Bioorg. Med. Chem. Lett. 17, 640-644 (2007).
64. S. Noël et al., Synthesis and biological properties of conjugates between fluoroquinolones and a N3″-functionalized pyochelin. Org. Biomol. Chem. 9, 8288 (2011).
65. C. Ji, M. J. Miller, Siderophore-fluoroquinolone conjugates containing potential reduction-triggered linkers for drug release: synthesis and antibacterial activity. Biometals 28, 541-551 (2015).
66. S. Fardeau et al., Synthesis and antibacterial activity of catecholate-ciprofloxacin conjugates. Bioorg. Med. Chem. 22, 4049-4060 (2014).
67. C. Ji, M. J. Miller, Chemical syntheses and in vitro antibacterial activity of two desferrioxamine B-ciprofloxacin conjugates with potential esterase and phosphatase triggered drug release linkers. Bioorg. Med. Chem. 20, 3828-3836 (2012).
68. T. A. Wencewicz, U. Möllmann, T. E. Long, M. J. Miller, Is drug release necessary for antimicrobial activity of siderophore-drug conjugates? Syntheses and biological studies of the naturally occurring salmycin “Trojan Horse” antibiotics and synthetic desferridanoxamine-antibiotic conjugates. Biometals 22, 633-648 (2009).
69. S. R. Md-Saleh et al., Synthesis of citrate-ciprofloxacin conjugates. Bioorg. Med. Chem. Lett. 19, 1496-1498 (2009).
70. R. E. Juárez-Hernández, P. A. Miller, M. J. Miller, Syntheses of Siderophore-Drug Conjugates Using a Convergent Thiol-Maleimide System. ACS Med Chem Lett 3, 799-803 (2012).
71. T. A. Wencewicz, M. J. Miller, Biscatecholate-monohydroxamate mixed ligand siderophore-carbacephalosporin conjugates are selective sideromycin antibiotics that target Acinetobacter baumannii. J. Med. Chem. 56, 4044-4052 (2013).
72. T. A. Wencewicz, T. E. Long, U. Möllmann, M. J. Miller, Trihydroxamate siderophore-fluoroquinolone conjugates are selective sideromycin antibiotics that target Staphylococcus aureus. Bioconjug. Chem. 24, 473-486 (2013).
73. S. J. Milner et al., Staphyloferrin A as siderophore-component in fluoroquinolone-based Trojan horse antibiotics. Org. Biomol. Chem. 11, 3461 (2013).
74. A. Souto et al., Synthesis and antibacterial activity of conjugates between norfloxacin and analogues of the siderophore vanchrobactin. Bioorg. Med. Chem. 21, 295-302 (2013).
75. T. Zheng, E. M. Nolan, Evaluation of (acyloxy)alkyl ester linkers for antibiotic release from siderophore-antibiotic conjugates. Bioorg. Med. Chem. Lett. 25, 4987-4991 (2015).
76. W. Neumann, E. M. Nolan, Evaluation of a reducible disulfide linker for siderophore-mediated delivery of antibiotics. J Biol Inorg Chem 23, 1025-1036 (2018).
77. S. Carosso et al., Methodology for Monobactam Diversification: Syntheses and Studies of 4-Thiomethyl Substituted beta-Lactams with Activity against Gram-Negative Bacteria, Including Carbapenemase Producing Acinetobacter baumannii. J. Med. Chem. 60, 8933-8944 (2017).
78. W. Neumann, M. Sassone-Corsi, M. Raffatellu, E. M. Nolan, Esterase-Catalyzed Siderophore Hydrolysis Activates an Enterobactin-Ciprofloxacin Conjugate and Confers Targeted Antibacterial Activity. J. Am. Chem. Soc. 140, 5193-5201 (2018).
79. C. Peukert et al., Siderophore conjugation with cleavable linkers boosts the potency of RNA polymerase inhibitors against multidrug-resistant E. coli. Chem Sci 14, 5490-5502 (2023).
80. Anonymous, FDA approves new antibacterial drug to treat complicated urinary tract infections as part of ongoing efforts to address antimicrobial resistance. Case Medical Research 10.31525/cmr-21c764f (2019).
81. M. Merdanovic, T. Clausen, M. Kaiser, R. Huber, M. Ehrmann, Protein Quality Control in the Bacterial Periplasm. Annu. Rev. Microbiol. 65, 149-168 (2011).
82. R. Domalaon et al., Repurposed Antimicrobial Combination Therapy: Tobramycin-Ciprofloxacin Hybrid Augments Activity of the Anticancer Drug Mitomycin C Against Multidrug-Resistant Gram-Negative Bacteria. Front Microbiol 10, 1556 (2019).
83. B. W. Kwan, N. Chowdhury, T. K. Wood, Combatting bacterial infections by killing persister cells with mitomycin C. Environ. Microbiol. 17, 4406-4414 (2015).
84. Y. Li, R. Sousa, Expression and purification of E. coli BirA biotin ligase for in vitro biotinylation. Protein Expr. Purif. 82, 162-167 (2012).
85. G. Chen, S. S. Sidhu, Design and generation of synthetic antibody libraries for phage display. Methods Mol Biol 1131, 113-131 (2014).
86. S. S. Sidhu, H. B. Lowman, B. C. Cunningham, J. A. Wells, Phage display for selection of novel binding peptides. Methods Enzymol. 328, 333-363 (2000).

### TABLES

TABLE 1
MIC comparison of solithromycin with siderophore-solithromycin conjugates that contain the optimized
LEAELG linker. Non-cleavable D-linker conjugate has minimal activity, while L-linker conjugate
shows similar or improved activity relative to solithromycin. These data support controlled
and effective release of solithromycin from LEAELG linker in 9 Gram-negative strains.

| | E. | | E | | K. | | | S. | |
|---|---|---|---|---|---|---|---|---|---|
| | coli | E. | aerogenes | A | pneumoniae | K. | A. | typhi | S. |
| Compound | K12 | coli | ATCC | baumannii | MGH | pneumoniae | baumannii | ATCC | enterica |
| (μM) | MG1655 | DCO | 13048 | WC487 | 78578 | KPPR1 | 5075 | 700931 | 14028s |
| solithromycin | <1 | <1 | 8 | 4 | 4 | 4 | 8 | 8 | 8 |
| HMM | <0.5 | <0.5 | 2 | 8 | <0.05 | 1 | 14 | 2 | 2 |
| siderophore- | | | | | | | | | |
| LEAELG- | | | | | | | | | |
| solithromycin | | | | | | | | | |
| HMM | 15 | 8 | >31 | 31 | >31 | >31 | >28 | >31 | >31 |
| siderophore- | | | | | | | | | |
| leaelg- | | | | | | | | | |
| solithromycin | | | | | | | | | |

TABLE 2
FRET pair conjugates prepared by the General procedure
for FRET Pair Conjugates via SPPS

| Compound | | calculated | found [M + H]⁺ |
|---|---|---|---|
| Number | MCA-Linker-DNP | exact mass | LCMS positive mode |
| 1A | MCA-HCAGFTP (SEQ | 1155.41 | 1156.5 |
| | ID NO: 28)-DNP | | |
| 2A | MCA-HVADDQG (SEQ | 1164.4 | 1165.5 |
| | ID NO: 91)-DNP | | |
| 3A | MCA-VQQNPCQ (SEQ | 1239.46 | 1240.4 |
| | ID NO: 30)-DNP | | |
| 4A | MCA-ENLWSPD (SEQ | 1283.47 | 1284.4 |
| | ID NO: 21)-DNP | | |
| 5A | MCA-LEHELGN(SEQ | 1234.49 | 1235.7 |
| | ID NO: 7)-DNP | | |
| 6A | MCA-YQSANAG(SEQ | 1133.41 | 1134.5 |
| | ID NO: 22)-DNP | | |
| 7A | MCA-NVPEHGT(SEQ | 1176.45 | 1177.6 |
| | ID NO: 23)-DNP | | |
| 8A | MCA-SGIEMYN(SEQ | 1236.44 | 1237.5 |
| | ID NO: 25)-DNP | | |
| 9A | MCA-LGVDPGS(SEQ | 1067.42 | 1068.6 |
| | ID NO: 26)-DNP | | |
| 10A | MCA-ENTMQCC(SEQ | 1251.36 | 1252.5 |
| | ID NO: 27)-DNP | | |
| 11A | MCA-NGACDAI(SEQ | 1086.37 | 1087.4 |
| | ID NO: 31)-DNP | | |
| 12A | MCA-MLPDHGI(SEQ | 1205.48 | 1206.6 |
| | ID NO: 32)-DNP | | |
| 13A | MCA-LGEVSAL(SEQ | 1111.48 | 1112.7 |
| | ID NO: 33)-DNP | | |
| 14A | MCA-AVASDSV(SEQ | 1071.41 | 1072.5 |
| | ID NO: 34)-DNP | | |
| 15A | MCA-GDNQLHN(SEQ | 1220.45 | 1221.5 |
| | ID NO: 36)-DNP | | |
| 16A | MCA-CSTLSPT(SEQ | 1131.42 | 1132.5 |
| | ID NO: 37)-DNP | | |
| 17A | MCA-HFPENAQ(SEQ | 1265.47 | 1266.6 |
| | ID NO: 38)-DNP | | |
| 18A | MCA-DAVNGEC(SEQ | 1130.36 | 1131.6 |
| | ID NO: 53)-DNP | | |
| 19A | MCA-SADLNNQ(SEQ | 1184.44 | 1185.6 |
| | ID NO: 40)-DNP | | |
| 20A | MCA-SALSNES(SEQ | 1130.42 | 1131.6 |
| | ID NO: 41)-DNP | | |
| 21A | MCA-QAPAPTS(SEQ | 1094.43 | 1095.6 |
| | ID NO: 42)-DNP | | |
| 22A | MCA-SANTPMT(SEQ | 1144.41 | 1145.6 |
| | ID NO: 43)-DNP | | |
| 23A | MCA-DSYYSGF(SEQ | 1261.42 | 1262.6 |
| | ID NO: 44)-DNP | | |
| 24A | MCA-STFDGGN(SEQ | 1120.37 | 1121.5 |
| | ID NO: 45)-DNP | | |
| 25A | MCA-DGSVEMT(SEQ | 1161.39 | 1162.3 |
| | ID NO: 46)-DNP | | |

TABLE 3
ACC conjugates prepared by the General procedure for the
synthesis of ACC Conjugates:

| Compound | | calculated | found ESI |
|---|---|---|---|
| Number | | exact mass | LCMS [M + H]⁺ |
| 1B | Ac-LELELG(SEQ ID | 914.44 | 915.7 |
| | NO: 66)-ACC | | |
| 2B | Ac-LESELG(SEQ ID | 888.39 | 889.7 |
| | NO: 70)-ACC | | |
| 3B | Ac-LEQELG(SEQ ID | 872.39 | 873.7 |
| | NO: 68)-ACC | | |
| 4B | Ac-LEIELG(SEQ ID | 914.44 | 915.5 |
| | NO: 64)-ACC | | |
| 5B | Ac-LEKELG(SEQ ID | 929.45 | 930.7 |
| | NO: 65)-ACC | | |
| 6B | Ac-LEGELG(SEQ ID | 858.38 | 859.6 |
| | NO: 58)-ACC | | |
| 7B | Ac-LEPELG(SEQ ID | 898.41 | 899.7 |
| | NO: 74)-ACC | | |
| 8B | Ac-LECELG(SEQ ID | 904.36 | 905.6 |
| | NO: 59)-ACC | | |
| 9B | Ac-LEDELG(SEQ ID | 916.38 | 917.7 |
| | NO: 60)-ACC | | |
| 10B | Ac-LEEELG(SEQ ID | 930.40 | 931.7 |
| | NO: 62)-ACC | | |
| 11B | Ac-LEFELG(SEQ ID | 948.42 | 949.7 |
| | NO: 61)-ACC | | |
| 12B | Ac-LEHELG(SEQ ID | 938.41 | 939.7 |
| | NO: 63)-ACC | | |
| 13B | Ac-LEMELG(SEQ | 932.39 | 933.7 |
| | ID NO: 57)-ACC | | |
| 14B | Ac-LENELG(SEQ ID | 915.40 | 916.7 |
| | NO: 67)-ACC | | |
| 15B | Ac-LERELG(SEQ ID | 957.46 | 958.8 |
| | NO: 69)-ACC | | |
| 16B | Ac-LETELG(SEQ ID | 902.40 | 903.7 |
| | NO: 71)-ACC | | |
| 17B | Ac-LEVELG(SEQ ID | 900.42 | 901.7 |
| | NO: 72)-ACC | | |
| 18B | Ac-LEWELG(SEQ | 987.43 | 988.8 |
| | ID NO: 73)-ACC | | |
| 19B | Ac-LEYELG(SEQ ID | 964.42 | 965.8 |
| | NO: 74)-ACC | | |
| 20B | Ac-LEAELG(SEQ ID | 872.39 | 873.1 |
| | NO: 3)-ACC | | |
| 21B | Ac-AEHELG-ACC | 896.37 | 897.2 |
| | (SEQ ID NO: 47) | | |
| 22B | Ac-LAHELG- | 880.41 | 880.8 |
| | ACC(SEQ ID NO: 48) | | |
| 23B | Ac-LEHALG-ACC | 880.41 | 880.9 |
| | (SEQ ID NO: 50) | | |
| 24B | Ac-LEHEAG-ACC | 896.37 | 897.2 |
| | (SEQ ID NO: 51) | | |
| 25B | Ac-LEHELA-ACC | 952.43 | 953.2 |
| | (SEQ ID NO: 52) | | |
| 26B | Ac-LEHELGN-ACC | 1052.46 | 1053.9 |
| | (SEQ ID NO: 8) | | |
| 27B | Ac-LEHE-ACC (SEQ | 768.31 | 769.3 |
| | ID NO: 11) | | |
| 28B | Ac-LEHEL-ACC | 881.39 | 882.4 |
| | (SEQ ID NO: 10) | | |
| 29B | Ac-LEH-ACC (SEQ | 639.27 | 640.3 |
| | ID NO: 12) | | |
| 30B | Ac-LE-ACC | 502.21 | 503.3 |
| 31B | Ac-L-ACC | 373.16 | 374.1 |

TABLE 4
HMM Siderophore-Linker-Siderophore and HMM
Siderophore-Linker-Mitomycin C conjugates:

| Compound | | calculated | found |
|---|---|---|---|
| Number | | exact mass | (ESI LCMS) |
| 1C | HMM siderophore- | 1856.91 | [M + H] 1857.4 |
| | LEAELG(SEQ ID NO: 3)- | | |
| | solithromycin | | |
| 2C | HMM siderophore-leaelG- | 1856.91 | [M + H] 1857.5 |
| | solithromycin | | |
| 3C | HMM siderophore- | 1922.94 | [M + H] 1923.8 |
| | LEHELG(SEQ ID NO: 1)- | | |
| | solithromycin | | |
| 4C | HMM siderophore-lehelG(SEQ | 1922.94 | [M + H] 1923.5 |
| | ID NO: 1)-solithromycin | | |
| 5C | HMM siderophore- | 1798.91 | [M + H] 1799.9 |
| | LEAALG(SEQ ID NO: 4)- | | |
| | solithromycin | | |
| 6C | HMM siderophore-leaalG(SEQ | 1798.91 | [M + H] 1799.8 |
| | ID NO: 4)-solithromycin | | |
| 7C | HMM siderophore- | 1864.93 | [M + H] 1865.6 |
| | LEHALG(SEQ ID NO: 2)- | | |
| | solithromycin | | |
| 8C | HMM siderophore-lehalG(SEQ | 1864.93 | [M + H] 1865.7 |
| | ID NO: 2)-solithromycin | | |
| 9C | HMM siderophore-LEHE(SEQ | 1752.83 | [M + H] 1753.6 |
| | ID NO: 13)-solithromycin | | |
| 10C | HMM siderophore-lehe(SEQ ID | 1752.83 | [M + H] 1753.3 |
| | NO: 13)-solithromycin | | |
| 11C | HMM siderophore- | 1412.59 | [M + H] 1413.3 |
| | LEHELG(SEQ ID NO: 1)- | | |
| | mitomycin C | | |
| 12C | HMM siderophore-lehelG(SEQ | 1412.59 | [M + H] 1413.4 |
| | ID NO: 1)-mitomycin C | | |
| 13C | HMM siderophore- | 1346.56 | [M − H] 1345.8 |
| | LEAELG(SEQ ID NO: 3)- | | |
| | mitomycin C | | |

SEQUENCES

| SEQ | | |
|---|---|---|
| ID | | |
| NO | Sequence | Features |
| 1 | LEHELG | L-LEHELG or D-LEHELG |
| 2 | LEHALG | L-LEHALG or D-LEHALG |
| 3 | LEAELG | L-LEAELG or D-LEAELG |
| 4 | LEAALG | L-LEAALG or D-LEAALG |
| 5 | LEXELG | L-LEXELG or D-LEXELG |
| 6 | LEAELX | L-LEAELX or D-LEAELX |
| 7 | LEHELGN | Linker |
| 8 | Ac-LEHELGN-ACC | Ac- refers to acetylation; -ACC refers |
| | | to fluorescent probe 7-amino-4- |
| | | carbamoylmethylcoumarin |
| 9 | Ac-LEHELG-ACC | Ac- refers to acetylation; -ACC refers |
| | | to fluorescent probe 7-amino-4- |
| | | carbamoylmethylcoumarin |
| 10 | Ac-LEHEL-ACC | Ac- refers to acetylation; -ACC refers |
| | | to fluorescent probe 7-amino-4- |
| | | carbamoylmethylcoumarin |
| 11 | Ac-LEHE-ACC | Ac- refers to acetylation; -ACC refers |
| | | to fluorescent probe 7-amino-4- |
| | | carbamoylmethylcoumarin |
| 12 | Ac-LEH-ACC | Ac- refers to acetylation; -ACC refers |
| | | to fluorescent probe 7-amino-4- |
| | | carbamoylmethylcoumarin |
| 13 | LEHE | Linker |
| 14 | WSPKYM | Linker |
| 15 | Ac-WSPKYM-ACC | Ac- refers to acetylation; -ACC refers |
| | | to fluorescent probe 7-amino-4- |
| | | carbamoylmethylcoumarin |
| 16 | TPTDPPTTPPT | Linker |
| 17 | GGSGG | Spacer/Linker |
| 18 | GGSAA | Spacer/Linker |
| 19 | GGS | Spacer/Linker |
| 20 | GGTPTDPPTTPPT | Linker |
| 21 | ENLWSPD | Linker |
| 22 | YQSANAG | Linker |
| 23 | NVPEHGT | Linker |
| 24 | AEHELG | Linker |
| 25 | SGIEMYN | Linker |
| 26 | LGVDPGS | Linker |
| 27 | ENTMQCC | Linker |
| 28 | HCAGFTP | Linker |
| 29 | HVADDQC | Linker |
| 30 | VQQNPCQ | Linker |
| 31 | NGACDAI | Linker |
| 32 | MLPDHGI | Linker |
| 33 | LGEVSAL | Linker |
| 34 | AVASDSV | Linker |
| 35 | LAHELG | Linker |
| 36 | GDNQLHN | Linker |
| 37 | CSTLSPT | Linker |
| 38 | HFPENAQ | Linker |
| 39 | DEVNGEC | Linker |
| 40 | SADLNNQ | Linker |
| 41 | SALSNES | Linker |
| 42 | QAPAPTS | Linker |
| 43 | SANTPMT | Linker |
| 44 | DSYYSGF | Linker |
| 45 | STFDGGN | Linker |
| 46 | DGSVEMT | Linker |
| 47 | Ac-AEHELG-ACC | Ac- refers to acetylation; -ACC refers |
| | | to fluorescent probe 7-amino-4- |
| | | carbamoylmethylcoumarin |
| 48 | Ac-LAHELG-ACC | Ac- refers to acetylation; -ACC refers |
| | | to fluorescent probe 7-amino-4- |
| | | carbamoylmethylcoumarin |
| 49 | Ac-LEAELG-ACC | Ac- refers to acetylation; -ACC refers |
| | | to fluorescent probe 7-amino-4- |
| | | carbamoylmethylcoumarin |
| 50 | Ac-LEHALG-ACC | Ac- refers to acetylation; -ACC refers |
| | | to fluorescent probe 7-amino-4- |
| | | carbamoylmethylcoumarin |
| 51 | Ac-LEHEAG-ACC | Ac- refers to acetylation; -ACC refers |
| | | to fluorescent probe 7-amino-4- |
| | | carbamoylmethylcoumarin |
| 52 | Ac-LEHELA-ACC | Ac- refers to acetylation; -ACC refers |
| | | to fluorescent probe 7-amino-4- |
| | | carbamoylmethylcoumarin |
| 53 | DAVNGEC | Linker |
| 54 | Ac-XXXXXXX-ACC | Ac- refers to acetylation; -ACC refers |
| | | to fluorescent probe 7-amino-4- |
| | | carbamoylmethylcoumarin; X refers |
| | | to any amino acid |
| 55 | Ac-XXXXXX-ACC | Ac- refers to acetylation; -ACC refers |
| | | to fluorescent probe 7-amino-4- |
| | | carbamoylmethylcoumarin; X refers |
| | | to any amino acid |
| 56 | Ac-XXXXX-ACC | Ac- refers to acetylation; -ACC refers |
| | | to fluorescent probe 7-amino-4- |
| | | carbamoylmethylcoumarin; X refers |
| | | to any amino acid |
| 57 | LEMELG | Linker |
| 58 | LEGELG | Linker |
| 59 | LECELG | Linker |
| 60 | LEDELG | Linker |
| 61 | LEFELG | Linker |
| 62 | LEEELG | Linker |
| 63 | LEHELG | Linker |
| 64 | LEIELG | Linker |
| 65 | LEKELG | Linker |
| 66 | LELELG | Linker |
| 67 | LENELG | Linker |
| 68 | LEQELG | Linker |
| 69 | LERELG | Linker |
| 70 | LESELG | Linker |
| 71 | LETELG | Linker |
| 72 | LEVELG | Linker |
| 73 | LEWELG | Linker |
| 74 | LEPELG | Linker |
| 75 | LEYELG | Linker |
| 76 | LEXE | Linker |
| 77 | TTG TTC CTT TCT ATT CTC ACA | gblock DNA sequence |
| | GTG CAC TTA TGG GCC TGA ACG | |
| | ATA TTT TTG AAG CGC AGA AAA | |
| | TTG AAT GGC ATG AAG GCG GCA | |
| | GCC TCG AGT TTG AAG CGC AGG | |
| | GTG GTA GTG CGG CCG CAC ATC | |
| | ATC ATC ACC | |
| 78 | MGLNDIFEAQKIEWHEGGSLEFEA | gblock amino acid sequence |
| | QGGSAAAHHHHHHGAAEQKLISE | |
| | EDLNG | |
| 79 | IEWHEGGSGGXXXXXXXGGSAA | Protein sequence containing Reverse |
| | AHHH | complement of unique barcode ; X = |
| | | any amino acid |
| 80 | TGA TGA TGA TGT GCG GCC GCA | primer sequence; M = adenosine or |
| | CTA CCA CCNMNNMNNM NNMNNM | cytosine; N = adenosine, cytosine, |
| | NNMNNA CCA CCG CTG CCG CCT | guanosine, or thymine |
| | TCA TGC CAT TCA AT | |
| 81 | GGXXXXXXX | AviTag (X = any amino acid) |
| 82 | GAGTTCAGACGTGTGCTCTTCCACA | Forward Primer |
| | CTCTTTCCCATGGTGATGATGATGTG | |
| | CGG | |
| 83 | AATGATACGGCGACCACCGAGATCT | Reverse Primer |
| | ACACGCAGAAATTGAATGGCATGA | |
| 84 | AATGATACGGCGACCACCGAGATCTA | Forward barcoding primer |
| | CACGCAGAAAATTGAATGGCATGA | |
| 85 | CAAGCAGAAGACGGCATACGAGATX | Reverse Barcoding primer; X = |
| | XXXXXXGTGACTGGAGTTCAGACGT | adenosine, cytosine, guanosine, or |
| | GTGCTCTTCC | thymine |
| 86 | XXXXXXX | Reverse Complement of unique |
| | | barcode; X = adenosine, cytosine, |
| | | guanosine, or thymine |
| 87 | AATGATACGGCGACCACC | Forward Primer |
| 88 | CAAGCAGAAGACGGCATAC | Reverse Primer |
| 89 | GACCACCGAGATCTACACGCAGAAA | Read1 Primer |
| | ATTGAATGGCATGA | |
| 90 | AATGATACGGCGACCACCGAGATCTA | Barcode construct; X = adenosine, |
| | CACGCAGAAAATTGAATGGCATGAAG | cytosine, guanosine, or thymine |
| | GCGGCAGCGGTGGTXXXXXXXX | |
| | XXXXXXXXXXXXXXGGTGGT | |
| | AGTGCGGCCGCACATCATCATCACCAT | |
| | GGGAAAGAGTGTGGAAGAGCACACG | |
| | TCTGAACTCCAGTCACXXXXXXXATC | |
| | TCGTATGCCGTCTTCTGCTTG | |
| 91 | HVADDQG | Linker |
| 92 | M-GLNDIFEAQKIEWHE-GGSGG- | Full protein sequence of phage |
| | XXXXXXX-GGS-AAAHHHHHH-GAA- | library; g3p = gene 3 protein and |
| | EQKLISEEDLNGAA-g3p | X-any amino acid |

In an amino acid sequence, X refers to any chiral amino acid including, but not limited to alanine, methionine, glycine, cysteine, aspartic acid, phenylalanine, glutamic acid, histidine, isoleucine, lysine, leucine, asparagine, glutamic acid, arginine, serine, threonine, valine, tryptophan, proline, and tyrosine. In a DNA sequence, X refers to any nucleotide including, but not limited to adenosine, cytosine, guanine, and thymine.

What is claimed is:

1. A method of screening for a protease-cleavable linker, the method comprising:
a) a negative selection process comprising:
i. incubating a substrate display library comprising one or more peptide sequences in a first complex biological matrix,
ii. generating a first cleaved phage-containing peptide sequence and an uncleaved phage-containing peptide sequence, and
iii. separating the uncleaved phage-containing peptide sequence from the first cleaved phage-containing peptide sequence;
b) a positive selection process comprising
i. contacting the uncleaved phage-containing peptide sequence with a second complex biological matrix, and
ii. producing a second cleaved phage-containing peptide sequence;
c) identifying a first nucleic acid sequence of the first cleaved phage-containing peptide sequence and identifying a second nucleic acid sequence of the second cleaved phage-containing peptide sequence,
d) comparing the first and second nucleic acid sequences to identify one or more protease-cleavable linkers; and
e) performing at least two iterations of steps a) through d).

2. The method of claim 1, wherein less than 10 iterations of steps a) through d) are performed.

3. The method of claim 1 or 2, wherein 2, 3, 4, 5, 6, 7, 8, or 9 iterations of steps a) through d) are performed.

4. The method of any one of claims 1-3, wherein the first complex biological matrix comprises healthy blood serum or plasma, healthy organ tissue homogenates, media comprising healthy organ tissue homogenates, healthy organoid homogenates derived from healthy cells, conditioned media comprising healthy organoids derived from healthy cells, healthy cell lines, conditioned media comprised of healthy cell lines, or combinations thereof.

5. The method of any one of claims 1-4, wherein the second complex biological matrix comprises cancer tissue homogenates, media comprising cancer tissue homogenates, cancer organoid homogenates, conditioned media comprising cancer organoid homogenates, malignant ascites, malignant cystic fluid, cancer cell lines, conditioned media comprising cancer cell lines, or combinations thereof.

6. The method of any one of claims 1-4, wherein the second complex biological matrix comprises periplasmic extracts isolated from Gram-negative bacteria, bacteria lysates from Gram-positive or Gram-negative bacteria, or combinations thereof.

7. The method of any one of claims 1-6, wherein the negative selection process comprises a control cell or a microenvironment comprising the control cell.

8. The method of any one of claims 1-7, wherein the positive selection process comprises a cell of interest or a microenvironment comprising the cell of interest.

9. The method of claim 8, wherein the cell of interest comprises a Gram-negative bacterium or a Gram-positive bacterium.

10. The method of claim 9, wherein the gram-negative bacterium comprises A. nosocomialis, P. aeruginosa, E. cloacae, E. coli, A. baumannii, E. aerogenes, K. pneumonia, S. typhi, H. influenzae, L. pneumophilia, V. cholerae, Y. enterocolitica, B. pertussis, B. melitensis, H. pylori, S. dysenteriae, C. jejuni, M. catarrhalis, B. cepacia, or S. enterica.

11. The method of claim 9, wherein the gram-positive bacterium comprises S. aureus, S. epidermidis, S. pneumoniae, S. pyogenes, C. difficile, C. perfringens, or L. monocytogenes.

12. The method of claim 8, wherein the cell of interest comprises a cancer cell.

13. The method of claim 12, wherein the cancer cell comprises hepatocellular carcinoma, cholangiocarcinoma, combined hepatocholangiocarcinoma, ovarian carcinoma, uterine carcinoma, triple negative breast cancer, glioblastoma, or pancreatic carcinoma.

14. The method of any one of claims 1-13, wherein the cell of interest is either the same or different in the at least two iterations of steps a) through e).

15. The method of any one of claims 1-14, wherein the first and second nucleic acid sequences encode the one or more protease-cleavable linkers.

16. The method of any one of claims 1-15, wherein the first and second nucleic acid sequence is identified using Next Generation Sequencing (NGS).

17. The method of any one of claims 1-16, further comprising identifying cleavage kinetics in the presence of the first complex biological matrix and the second complex biological matrix.

18. A compound comprising a targeting moiety, a therapeutic agent, and a cleavable linker, wherein the cleavable linker comprises a peptide sequence selected from SEQ ID NO: 1, SEQ ID NO: 2, SEQ ID NO: 3, SEQ ID NO: 4, SEQ ID NO: 5, SEQ ID NO: 6, or a chiral variant thereof.

19. The compound of claim 18, wherein the cleavable linker is directly or indirectly linked to the targeting moiety and the therapeutic agent.

20. The compound of claim 18 or 19, wherein the targeting moiety comprises a siderophore.

21. The compound of any one of claims 18-20, wherein the targeting moiety comprises an HMM siderophore, an enterobactin, an aerobactin, a yersiniabactin, or derivatives thereof.

22. The compound of claim 21, wherein the HMM siderophore comprises Compound I

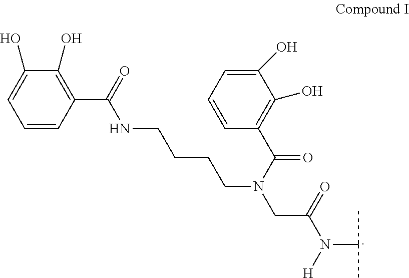

23. The compound of claim 21, wherein the enterobactin comprises Compound II

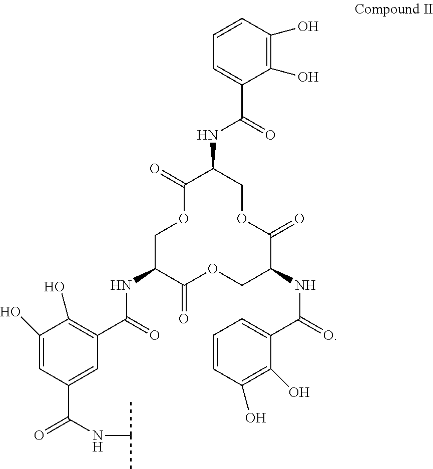

24. The compound of claim 21, wherein the aerobactin comprises Compound III

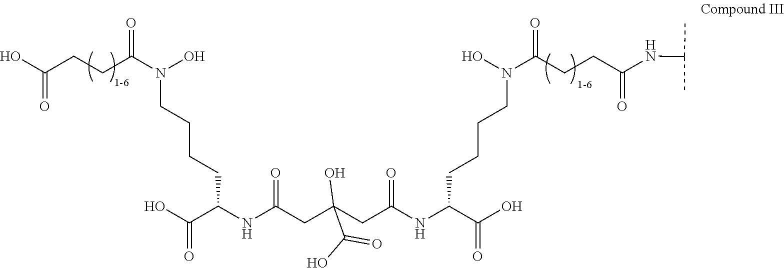

25. The compound of claim 21, wherein the yersiniabactin comprises Compound IV

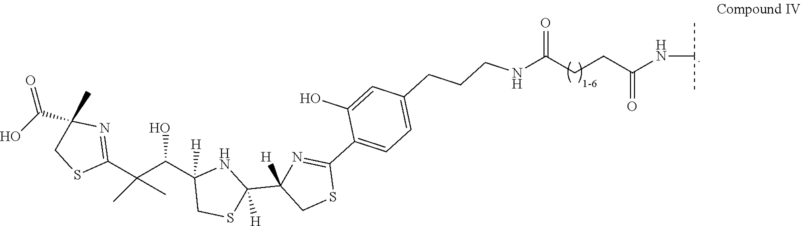

26. The compound of any one of claims 18-25, wherein the therapeutic agent comprises an antibiotic.

27. The compound of claim 26, wherein the antibiotic targets a cytoplasmic protein, a periplasmic protein, or a bacterial membrane protein.

28. The compound of claim 26 or 27, wherein the antibiotic comprises an amine- or aniline-containing antibiotic.

29. The compound of any one of claims 26-28, wherein the antibiotic comprises a macrolide antibiotic, oxazolidinone antibiotic, cyclic lipopeptide antibiotic, or fluoroquinolone antibiotic.

30. The compound of any one of claims 26-29, wherein the antibiotic comprises solithromycin, daptomycin, or eperezolid.

31. The compound of any one of claims 18-30, wherein the therapeutic agent comprises an anti-cancer chemotherapeutic agent.

32. The compound of claim 31, wherein the anti-cancer chemotherapeutic agent comprises an apoptosis agent, an anti-mitotic agent, a kinase inhibitor, an antimetabolite, an alkylating agent, paclitaxel, docetaxel, a bifunctional proteolysis-targeting chimera, or a combination thereof.

33. The compound of claim 31 or 32, wherein the anti-cancer chemotherapeutic agent comprises doxorubicin, mitomycin, or a combination thereof.
